SEQUENCE LISTING (105 sequences)

SEQ ID NO: 1 - protein, 7 aa, Artificial Sequence
  Synthetic Construct
  MISC_FEATURE (2)..(2): Xaa is bAla
  MISC_FEATURE (5)..(5): Xaa is bAla
RXRRXRR

SEQ ID NO: 2 - protein, 5 aa, Artificial Sequence
  Synthetic Construct
  MISC_FEATURE (2)..(2): Xaa is bAla
  MISC_FEATURE (4)..(4): Xaa is bAla
RXRXR

SEQ ID NO: 3 - protein, 4 aa, Artificial Sequence
  Synthetic Construct
  MISC_FEATURE (2)..(2): Xaa is bAla
RXRR

SEQ ID NO: 4 - protein, 6 aa, Artificial Sequence
  Synthetic Construct
  MISC_FEATURE (2)..(2): Xaa is bAla
  MISC_FEATURE (5)..(5): Xaa is bAla
RXRRXR

SEQ ID NO: 5 - protein, 6 aa, Artificial Sequence
  Synthetic Construct
  MISC_FEATURE (3)..(3): Xaa is bAla
  MISC_FEATURE (5)..(5): Xaa is bAla
RRXRXR

SEQ ID NO: 6 - protein, 5 aa, Artificial Sequence
  Synthetic Construct
  MISC_FEATURE (2)..(2): Xaa is bAla
  MISC_FEATURE (5)..(5): Xaa is bAla
RXRRX

SEQ ID NO: 7 - protein, 4 aa, Artificial Sequence
  Synthetic Construct
  MISC_FEATURE (1)..(1): Xaa is bAla
  MISC_FEATURE (3)..(3): Xaa is bAla
XRXR

SEQ ID NO: 8 - protein, 5 aa, Artificial Sequence
  Synthetic Construct
  MISC_FEATURE (2)..(2): Xaa is bAla
  MISC_FEATURE (4)..(4): Xaa is bAla
RXHXH

SEQ ID NO: 9 - protein, 5 aa, Artificial Sequence
  Synthetic Construct
  MISC_FEATURE (2)..(2): Xaa is bAla
  MISC_FEATURE (4)..(4): Xaa is bAla
HXHXR

SEQ ID NO: 10 - protein, 7 aa, Artificial Sequence
  Synthetic Construct
  MISC_FEATURE (2)..(2): Xaa is bAla
  MISC_FEATURE (5)..(5): Xaa is bAla
RXRHXHR

SEQ ID NO: 11 - protein, 7 aa, Artificial Sequence
  Synthetic Construct
  MISC_FEATURE (2)..(2): Xaa is bAla
  MISC_FEATURE (4)..(4): Xaa is bAla
  MISC_FEATURE (5)..(5): Xaa is bAla
RXRXXHR

SEQ ID NO: 12 - protein, 6 aa, Artificial Sequence
  Synthetic Construct
  MISC_FEATURE (2)..(2): Xaa is bAla
  MISC_FEATURE (5)..(5): Xaa is bAla
RXRRXH

SEQ ID NO: 13 - protein, 6 aa, Artificial Sequence
  Synthetic Construct
  MISC_FEATURE (2)..(2): Xaa is bAla
  MISC_FEATURE (5)..(5): Xaa is bAla
HXRRXR

SEQ ID NO: 14 - protein, 5 aa, Artificial Sequence
  Synthetic Construct
  MISC_FEATURE (2)..(2): Xaa is bAla
  MISC_FEATURE (4)..(4): Xaa is bAla
HXHXH

SEQ ID NO: 15 - protein, 4 aa, Artificial Sequence
  Synthetic Construct
  MISC_FEATURE (1)..(1): Xaa is bAla
  MISC_FEATURE (3)..(3): Xaa is bAla
XHXH

SEQ ID NO: 16 - protein, 5 aa, Artificial Sequence
  Synthetic Construct
  MISC_FEATURE (1)..(1): Xaa is bAla
  MISC_FEATURE (3)..(3): Xaa is bAla
  MISC_FEATURE (5)..(5): Xaa is bAla
XRXSX

SEQ ID NO: 17 - protein, 5 aa, Artificial Sequence
  Synthetic Construct
  MISC_FEATURE (1)..(1): Xaa is bAla
  MISC_FEATURE (3)..(3): Xaa is bAla
  MISC_FEATURE (4)..(4): Xaa is Hyp
  MISC_FEATURE (5)..(5): Xaa is bAla
XRXXX

SEQ ID NO: 18 - protein, 6 aa, Artificial Sequence
  Synthetic Construct
  MISC_FEATURE (2)..(2): Xaa is Hyp
  MISC_FEATURE (4)..(4): Xaa is Hyp
  MISC_FEATURE (6)..(6): Xaa is bAla
RXHXHX

SEQ ID NO: 19 - protein, 6 aa, Artificial Sequence
  Synthetic Construct
  MISC_FEATURE (2)..(2): Xaa is Hyp
  MISC_FEATURE (5)..(5): Xaa is Hyp
RXRRXR

SEQ ID NO: 20 - protein, 5 aa, Artificial Sequence
  Synthetic Construct
YQFLI

SEQ ID NO: 21 - protein, 5 aa, Artificial Sequence
  Synthetic Construct
FQILY

SEQ ID NO: 22 - protein, 5 aa, Artificial Sequence
  Synthetic Construct
ILFQY

SEQ ID NO: 23 - protein, 4 aa, Artificial Sequence
  Synthetic Construct
FQIY

SEQ ID NO: 24 - protein, 8 aa, Artificial Sequence
  Synthetic Construct
WWWWWPWW

SEQ ID NO: 25 - protein, 4 aa, Artificial Sequence
  Synthetic Construct
WPWW

SEQ ID NO: 26 - protein, 4 aa, Artificial Sequence
  Synthetic Construct
WWPW

SEQ ID NO: 27 - protein, 17 aa, Artificial Sequence
  Synthetic Construct
  MISC_FEATURE (2)..(2): Xaa is bAla
  MISC_FEATURE (5)..(5): Xaa is bAla
  MISC_FEATURE (14)..(14): Xaa is bAla
  MISC_FEATURE (16)..(16): Xaa is bAla
RXRRXRRFQILYRXRXR

SEQ ID NO: 28 - protein, 16 aa, Artificial Sequence
  Synthetic Construct
  MISC_FEATURE (2)..(2): Xaa is bAla
  MISC_FEATURE (5)..(5): Xaa is bAla
  MISC_FEATURE (14)..(14): Xaa is bAla
RXRRXRRFQILYRXRR

SEQ ID NO: 29 - protein, 17 aa, Artificial Sequence
  Synthetic Construct
  MISC_FEATURE (2)..(2): Xaa is bAla
  MISC_FEATURE (5)..(5): Xaa is bAla
  MISC_FEATURE (14)..(14): Xaa is bAla
  MISC_FEATURE (16)..(16): Xaa is bAla
RXRRXRFQILYRRXRXR

SEQ ID NO: 30 - protein, 17 aa, Artificial Sequence
  Synthetic Construct
  MISC_FEATURE (2)..(2): Xaa is bAla
  MISC_FEATURE (4)..(4): Xaa is bAla
  MISC_FEATURE (12)..(12): Xaa is bAla
  MISC_FEATURE (15)..(15): Xaa is bAla
RXRXRFQILYRXRRXRR

SEQ ID NO: 31 - protein, 17 aa, Artificial Sequence
  Synthetic Construct
  MISC_FEATURE (2)..(2): Xaa is bAla
  MISC_FEATURE (5)..(5): Xaa is bAla
  MISC_FEATURE (14)..(14): Xaa is bAla
  MISC_FEATURE (16)..(16): Xaa is bAla
RXRRXRRYQFLIRXRXR

SEQ ID NO: 32 - protein, 17 aa, Artificial Sequence
  Synthetic Construct
  MISC_FEATURE (2)..(2): Xaa is bAla
  MISC_FEATURE (5)..(5): Xaa is bAla
  MISC_FEATURE (14)..(14): Xaa is bAla
  MISC_FEATURE (16)..(16): Xaa is bAla
RXRRXRRILFQYRXRXR

SEQ ID NO: 33 - protein, 16 aa, Artificial Sequence
  Synthetic Construct
  MISC_FEATURE (2)..(2): Xaa is bAla
  MISC_FEATURE (5)..(5): Xaa is bAla
  MISC_FEATURE (13)..(13): Xaa is bAla
  MISC_FEATURE (15)..(15): Xaa is bAla
RXRRXRFQILYRXRXR

SEQ ID NO: 34 - protein, 16 aa, Artificial Sequence
  Synthetic Construct
  MISC_FEATURE (2)..(2): Xaa is bAla
  MISC_FEATURE (5)..(5): Xaa is bAla
  MISC_FEATURE (12)..(12): Xaa is bAla
  MISC_FEATURE (15)..(15): Xaa is bAla
RXRRXFQILYRXRRXR

SEQ ID NO: 35 - protein, 15 aa, Artificial Sequence
  Synthetic Construct
  MISC_FEATURE (2)..(2): Xaa is bAla
  MISC_FEATURE (5)..(5): Xaa is bAla
  MISC_FEATURE (12)..(12): Xaa is bAla
  MISC_FEATURE (14)..(14): Xaa is bAla
RXRRXRFQILYXRXR

SEQ ID NO: 36 - protein, 15 aa, Artificial Sequence
  Synthetic Construct
  MISC_FEATURE (2)..(2): Xaa is bAla
  MISC_FEATURE (5)..(5): Xaa is bAla
  MISC_FEATURE (12)..(12): Xaa is bAla
  MISC_FEATURE (14)..(14): Xaa is bAla
RXRRXFQILYRXRXR

SEQ ID NO: 37 - protein, 17 aa, Artificial Sequence
  Synthetic Construct
  MISC_FEATURE (2)..(2): Xaa is bAla
  MISC_FEATURE (5)..(5): Xaa is bAla
  MISC_FEATURE (14)..(14): Xaa is bAla
  MISC_FEATURE (16)..(16): Xaa is bAla
RXRRXRRFQILYRXHXH

SEQ ID NO: 38 - protein, 17 aa, Artificial Sequence
  Synthetic Construct
  MISC_FEATURE (2)..(2): Xaa is bAla
  MISC_FEATURE (5)..(5): Xaa is bAla
  MISC_FEATURE (14)..(14): Xaa is bAla
  MISC_FEATURE (16)..(16): Xaa is bAla
RXRRXRRFQILYHXHXR

SEQ ID NO: 39 - protein, 17 aa, Artificial Sequence
  Synthetic Construct
  MISC_FEATURE (2)..(2): Xaa is bAla
  MISC_FEATURE (5)..(5): Xaa is bAla
  MISC_FEATURE (14)..(14): Xaa is bAla
  MISC_FEATURE (16)..(16): Xaa is bAla
RXRRXRRFQILYHXRXH

SEQ ID NO: 40 - protein, 17 aa, Artificial Sequence
  Synthetic Construct
  MISC_FEATURE (2)..(2): Xaa is bAla
  MISC_FEATURE (5)..(5): Xaa is bAla
  MISC_FEATURE (14)..(14): Xaa is bAla
  MISC_FEATURE (16)..(16): Xaa is bAla
RXRRXRRYQFLIRXHXH

SEQ ID NO: 41 - protein, 17 aa, Artificial Sequence
  Synthetic Construct
  MISC_FEATURE (2)..(2): Xaa is bAla
  MISC_FEATURE (5)..(5): Xaa is bAla
  MISC_FEATURE (14)..(14): Xaa is bAla
  MISC_FEATURE (16)..(16): Xaa is bAla
RXRRXRRILFQYRXHXH

SEQ ID NO: 42 - protein, 17 aa, Artificial Sequence
  Synthetic Construct
  MISC_FEATURE (2)..(2): Xaa is bAla
  MISC_FEATURE (5)..(5): Xaa is bAla
  MISC_FEATURE (14)..(14): Xaa is bAla
  MISC_FEATURE (16)..(16): Xaa is bAla
RXRHXHRFQILYRXRXR

SEQ ID NO: 43 - protein, 17 aa, Artificial Sequence
  Synthetic Construct
  MISC_FEATURE (2)..(2): Xaa is bAla
  MISC_FEATURE (4)..(4): Xaa is bAla
  MISC_FEATURE (5)..(5): Xaa is bAla
  MISC_FEATURE (14)..(14): Xaa is bAla
  MISC_FEATURE (16)..(16): Xaa is bAla
RXRXXHRFQILYRXHXH

SEQ ID NO: 44 - protein, 16 aa, Artificial Sequence
  Synthetic Construct
  MISC_FEATURE (2)..(2): Xaa is bAla
  MISC_FEATURE (5)..(5): Xaa is bAla
  MISC_FEATURE (13)..(13): Xaa is bAla
  MISC_FEATURE (15)..(15): Xaa is bAla
RXRRXRFQILYRXHXH

SEQ ID NO: 45 - protein, 16 aa, Artificial Sequence
  Synthetic Construct
  MISC_FEATURE (2)..(2): Xaa is bAla
  MISC_FEATURE (5)..(5): Xaa is bAla
  MISC_FEATURE (13)..(13): Xaa is bAla
  MISC_FEATURE (15)..(15): Xaa is bAla
RXRRXRFQILYHXHXH

SEQ ID NO: 46 - protein, 16 aa, Artificial Sequence
  Synthetic Construct
  MISC_FEATURE (2)..(2): Xaa is bAla
  MISC_FEATURE (5)..(5): Xaa is bAla
  MISC_FEATURE (13)..(13): Xaa is bAla
  MISC_FEATURE (15)..(15): Xaa is bAla
RXRRXHFQILYRXHXH

SEQ ID NO: 47 - protein, 16 aa, Artificial Sequence
  Synthetic Construct
  MISC_FEATURE (2)..(2): Xaa is bAla
  MISC_FEATURE (5)..(5): Xaa is bAla
  MISC_FEATURE (13)..(13): Xaa is bAla
  MISC_FEATURE (15)..(15): Xaa is bAla
HXRRXRFQILYRXHXH

SEQ ID NO: 48 - protein, 15 aa, Artificial Sequence
  Synthetic Construct
  MISC_FEATURE (2)..(2): Xaa is bAla
  MISC_FEATURE (5)..(5): Xaa is bAla
  MISC_FEATURE (12)..(12): Xaa is bAla
  MISC_FEATURE (14)..(14): Xaa is bAla
RXRRXFQILYRXHXH

SEQ ID NO: 49 - protein, 15 aa, Artificial Sequence
  Synthetic Construct
  MISC_FEATURE (2)..(2): Xaa is bAla
  MISC_FEATURE (5)..(5): Xaa is bAla
  MISC_FEATURE (12)..(12): Xaa is bAla
  MISC_FEATURE (14)..(14): Xaa is bAla
RXRRXRFQILYXHXH

SEQ ID NO: 50 - protein, 16 aa, Artificial Sequence
  Synthetic Construct
  MISC_FEATURE (2)..(2): Xaa is bAla
  MISC_FEATURE (5)..(5): Xaa is bAla
  MISC_FEATURE (13)..(13): Xaa is bAla
  MISC_FEATURE (15)..(15): Xaa is bAla
RXRRXRYQFLIHXHXH

SEQ ID NO: 51 - protein, 16 aa, Artificial Sequence
  Synthetic Construct
  MISC_FEATURE (2)..(2): Xaa is bAla
  MISC_FEATURE (5)..(5): Xaa is bAla
  MISC_FEATURE (13)..(13): Xaa is bAla
  MISC_FEATURE (15)..(15): Xaa is bAla
RXRRXRILFQYHXHXH

SEQ ID NO: 52 - protein, 17 aa, Artificial Sequence
  Synthetic Construct
  MISC_FEATURE (2)..(2): Xaa is bAla
  MISC_FEATURE (5)..(5): Xaa is bAla
  MISC_FEATURE (14)..(14): Xaa is bAla
  MISC_FEATURE (16)..(16): Xaa is bAla
RXRRXRRFQILYHXHXH

SEQ ID NO: 53 - protein, 15 aa, Artificial Sequence
  Synthetic Construct
  MISC_FEATURE (2)..(2): Xaa is bAla
  MISC_FEATURE (5)..(5): Xaa is bAla
  MISC_FEATURE (12)..(12): Xaa is bAla
  MISC_FEATURE (14)..(14): Xaa is bAla
RXRRXRFQILYXRXS

SEQ ID NO: 54 - protein, 15 aa, Artificial Sequence
  Synthetic Construct
  MISC_FEATURE (2)..(2): Xaa is bAla
  MISC_FEATURE (5)..(5): Xaa is bAla
  MISC_FEATURE (12)..(12): Xaa is bAla
  MISC_FEATURE (14)..(14): Xaa is bAla
  MISC_FEATURE (15)..(15): Xaa is Hyp
RXRRXRFQILYXRXX

SEQ ID NO: 55 - protein, 15 aa, Artificial Sequence
  Synthetic Construct
  MISC_FEATURE (2)..(2): Xaa is bAla
  MISC_FEATURE (5)..(5): Xaa is bAla
  MISC_FEATURE (12)..(12): Xaa is bAla
  MISC_FEATURE (14)..(14): Xaa is Hyp
RXRRXRFQILYXRXR

SEQ ID NO: 56 - protein, 16 aa, Artificial Sequence
  Synthetic Construct
  MISC_FEATURE (3)..(3): Xaa is bAla
  MISC_FEATURE (6)..(6): Xaa is bAla
  MISC_FEATURE (13)..(13): Xaa is bAla
  MISC_FEATURE (15)..(15): Xaa is bAla
RRXRRXRFQILYXRXR

SEQ ID NO: 57 - protein, 15 aa, Artificial Sequence
  Synthetic Construct
  MISC_FEATURE (1)..(1): Xaa is bAla
  MISC_FEATURE (4)..(4): Xaa is bAla
  MISC_FEATURE (12)..(12): Xaa is bAla
  MISC_FEATURE (14)..(14): Xaa is bAla
XRRXRRFQILYXRXR

SEQ ID NO: 58 - protein, 13 aa, Artificial Sequence
  Synthetic Construct
  MISC_FEATURE (2)..(2): Xaa is bAla
  MISC_FEATURE (5)..(5): Xaa is bAla
  MISC_FEATURE (10)..(10): Xaa is bAla
  MISC_FEATURE (12)..(12): Xaa is bAla
RXRRXRWWWXRXR

SEQ ID NO: 59 - protein, 15 aa, Artificial Sequence
  Synthetic Construct
  MISC_FEATURE (2)..(2): Xaa is bAla
  MISC_FEATURE (5)..(5): Xaa is bAla
  MISC_FEATURE (12)..(12): Xaa is bAla
  MISC_FEATURE (14)..(14): Xaa is bAla
RXRRXRWWPWWXRXR

SEQ ID NO: 60 - protein, 14 aa, Artificial Sequence
  Synthetic Construct
  MISC_FEATURE (2)..(2): Xaa is bAla
  MISC_FEATURE (5)..(5): Xaa is bAla
  MISC_FEATURE (11)..(11): Xaa is bAla
  MISC_FEATURE (13)..(13): Xaa is bAla
RXRRXRWPWWXRXR

SEQ ID NO: 61 - protein, 14 aa, Artificial Sequence
  Synthetic Construct
  MISC_FEATURE (2)..(2): Xaa is bAla
  MISC_FEATURE (5)..(5): Xaa is bAla
  MISC_FEATURE (11)..(11): Xaa is bAla
  MISC_FEATURE (13)..(13): Xaa is bAla
RXRRXRWWPWXRXR

SEQ ID NO: 62 - protein, 15 aa, Artificial Sequence
  Synthetic Construct
  MISC_FEATURE (2)..(2): Xaa is bAla
  MISC_FEATURE (5)..(5): Xaa is bAla
  MISC_FEATURE (12)..(12): Xaa is bAla
  MISC_FEATURE (14)..(14): Xaa is bAla
RXRRXRRWWWRXRXR

SEQ ID NO: 63 - protein, 17 aa, Artificial Sequence
  Synthetic Construct
  MISC_FEATURE (2)..(2): Xaa is bAla
  MISC_FEATURE (5)..(5): Xaa is bAla
  MISC_FEATURE (14)..(14): Xaa is bAla
  MISC_FEATURE (16)..(16): Xaa is bAla
RXRRXRRWWPWWRXRXR

SEQ ID NO: 64 - protein, 16 aa, Artificial Sequence
  Synthetic Construct
  MISC_FEATURE (2)..(2): Xaa is bAla
  MISC_FEATURE (5)..(5): Xaa is bAla
  MISC_FEATURE (13)..(13): Xaa is bAla
  MISC_FEATURE (15)..(15): Xaa is bAla
RXRRXRRWPWWRXRXR

SEQ ID NO: 65 - protein, 16 aa, Artificial Sequence
  Synthetic Construct
  MISC_FEATURE (2)..(2): Xaa is bAla
  MISC_FEATURE (5)..(5): Xaa is bAla
  MISC_FEATURE (13)..(13): Xaa is bAla
  MISC_FEATURE (15)..(15): Xaa is bAla
RXRRXRRWWPWRXRXR

SEQ ID NO: 66 - protein, 16 aa, Artificial Sequence
  Synthetic Construct
  MISC_FEATURE (2)..(2): Xaa is bAla
  MISC_FEATURE (5)..(5): Xaa is bAla
  MISC_FEATURE (13)..(13): Xaa is bAla
  MISC_FEATURE (15)..(15): Xaa is bAla
RXRRXRRFQILYXRXR

SEQ ID NO: 67 - protein, 15 aa, Artificial Sequence
  Synthetic Construct
  MISC_FEATURE (2)..(2): Xaa is bAla
  MISC_FEATURE (5)..(5): Xaa is bAla
  MISC_FEATURE (14)..(14): Xaa is bAla
RXRRXRRFQILYRXR

SEQ ID NO: 68 - protein, 16 aa, Artificial Sequence
  Synthetic Construct
  MISC_FEATURE (1)..(1): Xaa is bAla
  MISC_FEATURE (3)..(3): Xaa is bAla
  MISC_FEATURE (5)..(5): Xaa is bAla
  MISC_FEATURE (12)..(12): Xaa is bAla
  MISC_FEATURE (15)..(15): Xaa is bAla
XRXRXWWPWWRXRRXR

SEQ ID NO: 69 - protein, 16 aa, Artificial Sequence
  Synthetic Construct
  MISC_FEATURE (2)..(2): Xaa is bAla
  MISC_FEATURE (5)..(5): Xaa is bAla
  MISC_FEATURE (13)..(13): Xaa is bAla
  MISC_FEATURE (15)..(15): Xaa is bAla
RXRRXRRFQILYXHXH

SEQ ID NO: 70 - protein, 16 aa, Artificial Sequence
  Synthetic Construct
  MISC_FEATURE (2)..(2): Xaa is bAla
  MISC_FEATURE (5)..(5): Xaa is bAla
  MISC_FEATURE (13)..(13): Xaa is bAla
  MISC_FEATURE (15)..(15): Xaa is bAla
RXRRXRRFQIYRXHXH

SEQ ID NO: 71 - protein, 15 aa, Artificial Sequence
  Synthetic Construct
  MISC_FEATURE (2)..(2): Xaa is bAla
  MISC_FEATURE (5)..(5): Xaa is bAla
  MISC_FEATURE (12)..(12): Xaa is bAla
  MISC_FEATURE (14)..(14): Xaa is bAla
RXRRXRFQILYXRXH

SEQ ID NO: 72 - protein, 16 aa, Artificial Sequence
  Synthetic Construct
  MISC_FEATURE (2)..(2): Xaa is bAla
  MISC_FEATURE (5)..(5): Xaa is bAla
  MISC_FEATURE (13)..(13): Xaa is Hyp
  MISC_FEATURE (15)..(15): Xaa is Hyp
RXRRXRFQILYRXHXH

SEQ ID NO: 73 - protein, 16 aa, Artificial Sequence
  Synthetic Construct
  MISC_FEATURE (2)..(2): Xaa is Hyp
  MISC_FEATURE (5)..(5): Xaa is Hyp
  MISC_FEATURE (13)..(13): Xaa is bAla
  MISC_FEATURE (15)..(15): Xaa is bAla
RXRRXRFQILYRXHXH

SEQ ID NO: 74 - protein, 16 aa, Artificial Sequence
  Synthetic Construct
  MISC_FEATURE (2)..(2): Xaa is Hyp
  MISC_FEATURE (5)..(5): Xaa is Hyp
  MISC_FEATURE (13)..(13): Xaa is Hyp
  MISC_FEATURE (15)..(15): Xaa is Hyp
RXRRXRFQILYRXHXH

SEQ ID NO: 75 - protein, 14 aa, Artificial Sequence
  Synthetic Construct
  MISC_FEATURE (2)..(2): Xaa is bAla
  MISC_FEATURE (5)..(5): Xaa is bAla
  MISC_FEATURE (11)..(11): Xaa is bAla
  MISC_FEATURE (13)..(13): Xaa is bAla
RXRRXRWWWRXHXH

SEQ ID NO: 76 - protein, 14 aa, Artificial Sequence
  Synthetic Construct
  MISC_FEATURE (2)..(2): Xaa is bAla
  MISC_FEATURE (5)..(5): Xaa is bAla
  MISC_FEATURE (11)..(11): Xaa is bAla
  MISC_FEATURE (13)..(13): Xaa is bAla
RXRRXRWWPRXHXH

SEQ ID NO: 77 - protein, 14 aa, Artificial Sequence
  Synthetic Construct
  MISC_FEATURE (2)..(2): Xaa is bAla
  MISC_FEATURE (5)..(5): Xaa is bAla
  MISC_FEATURE (11)..(11): Xaa is bAla
  MISC_FEATURE (13)..(13): Xaa is bAla
RXRRXRPWWRXHXH

SEQ ID NO: 78 - protein, 16 aa, Artificial Sequence
  Synthetic Construct
  MISC_FEATURE (2)..(2): Xaa is bAla
  MISC_FEATURE (5)..(5): Xaa is bAla
  MISC_FEATURE (13)..(13): Xaa is bAla
  MISC_FEATURE (15)..(15): Xaa is bAla
RXRRXRWWPWWRXHXH

SEQ ID NO: 79 - protein, 15 aa, Artificial Sequence
  Synthetic Construct
  MISC_FEATURE (2)..(2): Xaa is bAla
  MISC_FEATURE (5)..(5): Xaa is bAla
  MISC_FEATURE (12)..(12): Xaa is bAla
  MISC_FEATURE (14)..(14): Xaa is bAla
RXRRXRWWPWRXHXH

SEQ ID NO: 80 - protein, 15 aa, Artificial Sequence
  Synthetic Construct
  MISC_FEATURE (2)..(2): Xaa is bAla
  MISC_FEATURE (5)..(5): Xaa is bAla
  MISC_FEATURE (12)..(12): Xaa is bAla
  MISC_FEATURE (14)..(14): Xaa is bAla
RXRRXRWPWWRXHXH

SEQ ID NO: 81 - protein, 15 aa, Artificial Sequence
  Synthetic Construct
  MISC_FEATURE (2)..(2): Xaa is bAla
  MISC_FEATURE (5)..(5): Xaa is bAla
  MISC_FEATURE (12)..(12): Xaa is bAla
  MISC_FEATURE (14)..(14): Xaa is bAla
RXRRXRRWWWRXHXH

SEQ ID NO: 82 - protein, 17 aa, Artificial Sequence
  Synthetic Construct
  MISC_FEATURE (2)..(2): Xaa is bAla
  MISC_FEATURE (5)..(5): Xaa is bAla
  MISC_FEATURE (14)..(14): Xaa is bAla
  MISC_FEATURE (16)..(16): Xaa is bAla
RXRRXRRWWPWWRXHXH

SEQ ID NO: 83 - protein, 16 aa, Artificial Sequence
  Synthetic Construct
  MISC_FEATURE (2)..(2): Xaa is bAla
  MISC_FEATURE (5)..(5): Xaa is bAla
  MISC_FEATURE (13)..(13): Xaa is bAla
  MISC_FEATURE (15)..(15): Xaa is bAla
RXRRXRRWPWWRXHXH

SEQ ID NO: 84 - protein, 16 aa, Artificial Sequence
  Synthetic Construct
  MISC_FEATURE (2)..(2): Xaa is bAla
  MISC_FEATURE (5)..(5): Xaa is bAla
  MISC_FEATURE (13)..(13): Xaa is bAla
  MISC_FEATURE (15)..(15): Xaa is bAla
RXRRXRRWWPWRXHXH

SEQ ID NO: 85 - protein, 17 aa, Artificial Sequence
  Synthetic Construct
  MISC_FEATURE (3)..(3): Xaa is bAla
  MISC_FEATURE (6)..(6): Xaa is bAla
  MISC_FEATURE (14)..(14): Xaa is bAla
  MISC_FEATURE (16)..(16): Xaa is bAla
RRXRRXRFQILYRXHXH

SEQ ID NO: 86 - protein, 16 aa, Artificial Sequence
  Synthetic Construct
  MISC_FEATURE (1)..(1): Xaa is bAla
  MISC_FEATURE (4)..(4): Xaa is bAla
  MISC_FEATURE (13)..(13): Xaa is bAla
  MISC_FEATURE (15)..(15): Xaa is bAla
XRRXRRFQILYRXHXH

SEQ ID NO: 87 - protein, 16 aa, Artificial Sequence
  Synthetic Construct
  MISC_FEATURE (3)..(3): Xaa is bAla
  MISC_FEATURE (6)..(6): Xaa is bAla
  MISC_FEATURE (13)..(13): Xaa is bAla
  MISC_FEATURE (15)..(15): Xaa is bAla
RRXRRXRFQILYXHXH

SEQ ID NO: 88 - protein, 15 aa, Artificial Sequence
  Synthetic Construct
  MISC_FEATURE (1)..(1): Xaa is bAla
  MISC_FEATURE (4)..(4): Xaa is bAla
  MISC_FEATURE (12)..(12): Xaa is bAla
  MISC_FEATURE (14)..(14): Xaa is bAla
XRRXRRFQILYXHXH

SEQ ID NO: 89 - protein, 17 aa, Artificial Sequence
  Synthetic Construct
  MISC_FEATURE (2)..(2): Xaa is bAla
  MISC_FEATURE (5)..(5): Xaa is bAla
  MISC_FEATURE (14)..(14): Xaa is bAla
  MISC_FEATURE (16)..(16): Xaa is bAla
RXRRXHRFQILYRXHXH

SEQ ID NO: 90 - skipped

SEQ ID NO: 91 - DNA, 20 bp, Artificial Sequence
  Synthetic Construct
gctgctgtcctcagcaagtt

SEQ ID NO: 92 - DNA, 20 bp, Artificial Sequence
  Synthetic Construct
ctgaatgtggctgcaaagaa

SEQ ID NO: 93 - DNA, 21 bp, Artificial Sequence
  Synthetic Construct
gctgcccaataccaggtcaac

SEQ ID NO: 94 - DNA, 22 bp, Artificial Sequence
  Synthetic Construct
tggtgggagaaatgctgtatgc

SEQ ID NO: 95 - DNA, 23 bp, Artificial Sequence
  Synthetic Construct
gctcatggtcctcaagatctcac

SEQ ID NO: 96 - DNA, 20 bp, Artificial Sequence
  Synthetic Construct
gggtcagtgcctcagctttg

SEQ ID NO: 97 - RNA, 21 nt, Artificial Sequence
  Synthetic Construct
cugcugcugcugcugcugcug

SEQ ID NO: 98-100 - skipped (3 entries)

SEQ ID NO: 101 - protein, 15 aa, Artificial Sequence
  Synthetic Construct
  MISC_FEATURE (2)..(2): Xaa is bAla
  MISC_FEATURE (5)..(5): Xaa is bAla
  MISC_FEATURE (12)..(12): Xaa is Hyp
  MISC_FEATURE (14)..(14): Xaa is Hyp
RXRRXRFQILYXRXR

SEQ ID NO: 102 - protein, 15 aa, Artificial Sequence
  Synthetic Construct
  MISC_FEATURE (2)..(2): Xaa is Hyp
  MISC_FEATURE (5)..(5): Xaa is Hyp
  MISC_FEATURE (12)..(12): Xaa is bAla
  MISC_FEATURE (14)..(14): Xaa is bAla
RXRRXRFQILYXRXR

SEQ ID NO: 103 - protein, 15 aa, Artificial Sequence
  Synthetic Construct
  MISC_FEATURE (2)..(2): Xaa is Hyp
  MISC_FEATURE (5)..(5): Xaa is Hyp
  MISC_FEATURE (12)..(12): Xaa is Hyp
  MISC_FEATURE (14)..(14): Xaa is Hyp
RXRRXRFQILYXRXR

SEQ ID NO: 104 - protein, 13 aa, Artificial Sequence
  Synthetic Construct
  MISC_FEATURE (2)..(2): Xaa is bAla
  MISC_FEATURE (5)..(5): Xaa is bAla
  MISC_FEATURE (10)..(10): Xaa is bAla
  MISC_FEATURE (12)..(12): Xaa is bAla
RXRRXRWWWXRXR

SEQ ID NO: 105 - protein, 15 aa, Artificial Sequence
  Synthetic Construct
  MISC_FEATURE (2)..(2): Xaa is bAla
  MISC_FEATURE (5)..(5): Xaa is bAla
  MISC_FEATURE (12)..(12): Xaa is bAla
  MISC_FEATURE (14)..(14): Xaa is bAla
RXRRXRWWPWWXRXR

(19) United States
(12) Patent Application Publication
(10) Pub. No.: US 2024/0189434 A1
(43) Pub. Date: Jun. 13, 2024

(54) METHODS OF TREATING MYOTONIC DYSTROPHY TYPE 1 USING PEPTIDE-OLIGONUCLEOTIDE CONJUGATES

(71) Applicant: PepGen Inc., Cambridge, MA (US)

(72) Inventors: Caroline GODFREY, Oxford (GB); Sonia BRACEGIRDLE, Belsele (BE); Ashling HOLLAND, Boston, MA (US); Smita GUNNOO, Oxford (GB)

(21) Appl. No.: 18/281,700

(22) PCT Filed: Mar. 11, 2022
(86) PCT No.: PCT/US2022/020070

§ 371 Date: Sep. 12, 2023

Related U.S. Application Data
(60) Provisional application No. 63/160,710, filed on Mar. 12, 2021.

(51) Int. Cl. A61K 47/64; A61K 47/54; A61P 21/00; C12N 15/113

(52) U.S. Cl. CPC A61K 47/64; A61K 47/54; A61P 21/00; C12N 15/1137; C12N 2310/11; C12N 2310/3233; C12N 2310/3513; C12N 2320/35

(57) ABSTRACT

Disclosed are methods of treating a subject having myotonic dystrophy type 1 (DM1). The methods include administering a therapeutic regimen including a plurality of doses of a conjugate spaced at a time interval of at least 1 month, where the conjugate includes an oligonucleotide and a peptide covalently bonded or linked via a linker to the oligonucleotide, the peptide including a hydrophobic domain flanked by two cationic domains, each of the cationic domains including one of RBRRBRR (SEQ ID NO: 1), RBRBR (SEQ ID NO: 2), RBRR (SEQ ID NO: 3), RBRRBR (SEQ ID NO: 4), RRBRBR (SEQ ID NO: 5), RBRRB (SEQ ID NO: 6), BRBR (SEQ ID NO: 7), RBHBH (SEQ ID NO: 8), HBHBR (SEQ ID NO: 9), RBRHBHR (SEQ ID NO: 10), RBRBBHR (SEQ ID NO: 11), RBRRBH (SEQ ID NO: 12), HBRRBR (SEQ ID NO: 13), HBHBH (SEQ ID NO: 14), BHBH (SEQ ID NO: 15), BRBSB (SEQ ID NO: 16), BRB[Hyp]B (SEQ ID NO: 17), R[Hyp]H[Hyp]HB (SEQ ID NO: 18), and R[Hyp]RR[Hyp]R (SEQ ID NO: 19), and the hydrophobic domain including one of YQFLI (SEQ ID NO: 20), FQILY (SEQ ID NO: 21), ILFQY (SEQ ID NO: 22), FQIY (SEQ ID NO: 23), VWVW, WWPWW (SEQ ID NO: 24), WPWW (SEQ ID NO: 25), and VWVPW (SEQ ID NO: 26); and the oligonucleotide including a total of 12 to 40 contiguous nucleobases, where at least 9 contiguous nucleobases are complementary to a CUG repeat sequence.

### FIELD OF THE INVENTION

The invention relates to methods of treating myotonic dystrophy type 1 using peptide conjugates of antisense oligonucleotides.

### BACKGROUND

Antisense oligonucleotides have shown considerable promise for use in the treatment of neuromuscular diseases, exemplified by their ability to modulate splicing in both spinal muscular atrophy (SMA) and Duchenne muscular dystrophy (DMD). Triplet repeat expansion, also known as trinucleotide repeat expansion or microsatellite repeat expansion, underlies many diseases, and modulation of such expansions can have therapeutic implications. Antisense oligonucleotides can be used to interfere in the binding between proteins and RNA species implicated in the pathogenesis of disease.

However, therapeutic development of these promising antisense therapeutics has been hampered by poor tissue penetration and cellular uptake.

Myotonic dystrophy 1 (DM1) is caused by expanded CUG repeats in the 3′-untranslated region of the dystrophia myotonica-protein kinase (DMPK) transcript (Mahadevan et al., Science 255:1253-1255, 1992), the gene for which is located on the long arm of chromosome 19. Morpholino ASOs have been developed that are able to form stable RNA-morpholino heteroduplexes with DMPK transcripts carrying the CUG repeats. In this way, the ASOs block interactions between these abnormal RNA species and other proteins such as muscleblind-like 1 (MBNL1), which plays a fundamental role in the control of the splicing machinery. However, while silencing the toxic DMPK transcript and induction of a normalizing effect on aberrant pre-mRNA splicing using ASOs has been demonstrated in vitro, effective silencing in vivo has remained elusive due to inefficient tissue penetration and cellular uptake of ASOs (Leger et al., Nucleic Acid Therapeutics 23(2)109-117, 2013). Indeed, a Phase 1/2a clinical trial for treatment of DM1 in humans was conducted by lonis Pharmaceuticals (ClinicalTrials.gov, Identifier: NCT02312011, clinicaltrials.gov/ct2/show/NCT02312011). Accordingly, there remains an urgent need to improve the delivery of antisense oligonucleotides to provide an effective therapy to a disease that currently has no therapy.

The use of viruses as delivery vehicles has been suggested, however, this is limited due to the immunotoxicity of the viral coat protein and potential oncogenic effects. Alternatively, a range of non-viral delivery vectors have been developed, amongst which peptides have shown the most promise due to their small size, low toxicity, targeting specificity and ability of trans-capillary delivery of large bio-cargoes (Farkhani et al., Peptides 57:78-94, 2014; Kang et al., Curr. Pharm. Biotechnol. 15:220-230, 2014; and Pardridge, J. Cereb. Blood Flow Metab. 32:1959-1972, 2012). Several peptides have been reported for their ability to permeate cells either alone or carrying a bio-cargo (Farkhani et al. and Kang et al. supra).

In particular, PNA/PMO internalization peptides (Pips) have been developed which are arginine-rich CPPs that are included of two arginine-rich sequences separated by a central short hydrophobic sequence. These ‘Pip’ peptides were designed to improve serum stability whilst maintaining a high level of exon skipping, initially by attachment to a peptide nucleic acids (PNA) cargo. Further derivatives of these peptides were designed as conjugates of phosphorodiamidate morpholino oligomers (PMOs), which were shown to lead to body-wide skeletal muscle dystrophin production, and importantly also including the heart, following systemic administration in mice (Betts et al., Molecular Therapy—Nucleic Acids 1(8), e38, 2012).

For several years, cell-penetrating peptides (CPPs) have been conjugated to splice switching oligonucleotides, SSOs, (in particular charge neutral PMO and PNA) in order to enhance the cell delivery of such oligonucleotide analogues by effectively carrying them across cell membranes to reach their pre-mRNA target sites in the cell nucleus. It has been shown that PMO therapeutics conjugated to certain arginine-rich CPPs (known as peptide-PMOs or P-PMOs) can enhance dystrophin production in skeletal muscles following systemic administration in a mdx mouse model of DMD.

Alternative cell-penetrating peptides having a single arginine rich domain such as R₆Gly have also been produced. These CPPs have been used to produce peptide conjugates with reduced toxicities, but these conjugates exhibited low efficacy in comparison to the Pip peptides.

Accordingly, the currently available CPPs have not yet been demonstrated as suitable for use in human treatments for diseases such as DM1.

Despite the efforts of researchers to vary the sequence of the carrier for use in therapeutic conjugates, until now it has proved very difficult to produce a conjugate with both high efficacy in terms of therapeutic results and acceptable toxicity levels.

Therefore, there remains a need for conjugates to deliver oligonucleotides that exhibit reduced toxicity when administered systemically to patients whilst maintaining therapeutic effectiveness.

One or more aspects of the present invention is intended to solve at least this problem.

The challenge in the field of cell-penetrating peptide technology has been to de-couple efficacy and toxicity. The present inventors have now identified, synthesized and tested a number of improved CPPs having a particular structure according to the present invention which address at least this problem in the treatment of triplet repeat expansion disorders such as myotonic dystrophy type 1 (DM1).

These peptide conjugates maintain good levels of efficacy in skeletal muscles when tested in vitro and in vivo with a cargo oligonucleotide. Furthermore, these peptide conjugates demonstrate an improvement in efficacy compared with conjugates including previously available CPPs when used to deliver the same therapeutic cargo. At the same time, these peptide conjugates act effectively in vivo with reduced clinical signs in animal models of triplet repeat expansion disorders such as myotonic dystrophy type 1 (DM1) following systemic injection and lower toxicity as observed through measurement of biochemical markers. Crucially, the present peptide conjugates are demonstrated to show a surprisingly reduced toxicity following similar systemic injection into mice when compared with conjugates including previous CPPs. Accordingly, the peptide conjugates used in the invention offer improved suitability for use as a therapy for humans than previously available peptide conjugates and can be used as therapeutic conjugates for safe and effective treatment of human subjects.

### SUMMARY OF THE INVENTION

In general, the invention provides methods of treating a subject having myotonic dystrophy type 1 (DM1).

In one aspect, the method includes administering a therapeutic regimen including a plurality of doses of a conjugate spaced at a time interval of at least 1 month, where the conjugate includes an oligonucleotide and a peptide covalently bonded or linked via a linker to the oligonucleotide, the peptide including a hydrophobic domain flanked by two cationic domains, each of the cationic domains including one of RBRRBRR (SEQ ID NO: 1), RBRBR (SEQ ID NO: 2), RBRR (SEQ ID NO: 3), RBRRBR (SEQ ID NO: 4), RRBRBR (SEQ ID NO: 5), RBRRB (SEQ ID NO: 6), BRBR (SEQ ID NO: 7), RBHBH (SEQ ID NO: 8), HBHBR (SEQ ID NO: 9), RBRHBHR (SEQ ID NO: 10), RBRBBHR (SEQ ID NO: 11), RBRRBH (SEQ ID NO: 12), HBRRBR (SEQ ID NO: 13), HBHBH (SEQ ID NO: 14), BHBH (SEQ ID NO: 15), BRBSB (SEQ ID NO: 16), BRB[Hyp]B (SEQ ID NO: 17), R[Hyp]H[Hyp]HB (SEQ ID NO: 18), and R[Hyp]RR[Hyp]R (SEQ ID NO: 19), and the hydrophobic domain including one of YQFLI (SEQ ID NO: 20), FQILY (SEQ ID NO: 21), ILFQY (SEQ ID NO: 22), FQIY (SEQ ID NO: 23), WWW, WWPWW (SEQ ID NO: 24), WPWW (SEQ ID NO: 25), and WWPW (SEQ ID NO: 26); and the oligonucleotide including a total of 12 to 40 contiguous nucleobases, where at least 9 contiguous nucleobases are complementary to a CUG repeat sequence.

In some embodiments, the time interval is 1 to 6 months. In some embodiments, the time interval is 2 to 6 months. In some embodiments, the time interval is 3 to 6 months. In some embodiments, the time interval is 3 to 4 months. In some embodiments, the time interval is 4 to 6 months. In some embodiments, the time interval is 5 to 6 months. In some embodiments, the time interval is 1 month, 2 months, 3 months, 4 months, 5 months, or 6 months.

In some embodiments, the therapeutic regimen further includes administering a treatment initiation or loading regimen including administering the conjugate three or four times at an initiation interval of 2 weeks.

In some embodiments, the amount of conjugate administered at the same dose level each time.

In some embodiments, the oligonucleotide is 5′-[CAG]ₙ-3′, where n is an integer from 5 to 8. In some embodiments, the oligonucleotide is 5′-[CAG]₅-3′. In some embodiments, the oligonucleotide is 5′-[CAG]₆-3′. In some embodiments, the oligonucleotide is 5′-[CAG]₇-3′. In some embodiments, the oligonucleotide is 5′-[CAG]₈-3′.

In some embodiments, the oligonucleotide is 5′-[AGC]ₙ-3′, where n is an integer from 5 to 8. In some embodiments, the oligonucleotide is 5′-[AGC]₅-3′. In some embodiments, the oligonucleotide is 5′-[AGC]₆-3′. In some embodiments, the oligonucleotide is 5′-[AGC]₇-3′. In some embodiments, the oligonucleotide is 5′-[AGC]₈-3′.

In some embodiments, the oligonucleotide is 5′-[GCA]ₙ-3′, where n is an integer from 5 to 8. In some embodiments, the oligonucleotide is 5′-[GCA]₅-3′. In some embodiments, the oligonucleotide is 5′-[GCA]₆-3′. In some embodiments, the oligonucleotide is 5′-[GCA]₇-3′. In some embodiments, the oligonucleotide is 5′-[GCA]₈-3′.

In some embodiments, the peptide has the following amino acid sequence RBRRBRFQILYBRBR (SEQ ID NO: 35). In some embodiments, the peptide has the following amino acid sequence RBRRBRRFQILYRBHBH (SEQ ID NO: 37). In some embodiments, the peptide has the following amino acid sequence RBRRBRFQILYRBHBH (SEQ ID NO: 44).

In some embodiments, the peptide is bonded to the rest of the conjugate through its N-terminus. In some embodiments, the C-terminus of the peptide is —CONH₂. In some embodiments, the peptide is bonded to the rest of the conjugate through its C-terminus. In some embodiments, the peptide is acylated at its N-terminus.

In some embodiments, the conjugate is of the following structure:

[peptide]—[linker]—[oligonucleotide]

In some embodiments, the conjugate is of the following structure:

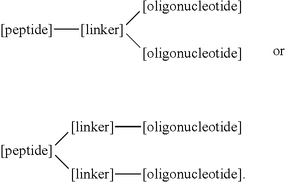

In some embodiments, the conjugate is of the following structure:

[peptide]—[linker]—[peptide]—[linker]—[oligonucleotide]

In some embodiments, each linker is independently of formula (I):

T₁—(CR¹R²)ₙ—T₂. (I)

where

T₁ is a divalent group for attachment to the peptide and is selected from the group consisting of —NH- and carbonyl;

T₂ is a divalent group for attachment to an oligonucleotide and is selected from the group consisting of —NH- and carbonyl;

n is 1, 2 or 3;

each R¹ is independently -Y¹-X¹-Z^{1,}

where
Y¹ is absent or —(CR^{A1}R^{A2})ₘ-, where m is 1, 2, 3 or 4, and R^{A1} and R^{A2} are each independently hydrogen, OH, or (1-2C)alkyl;
X¹ is absent, —O—, —C(O)—, —C(O)O—, —OC(O)—, —CH(OR^{A3})—, —N(R^{A3})—, —N(R^{A3})—C(O)—, —N(R^{A3})—C(O)O—, —C(O)—N(R^{A3})—, —N(R^{A3})C(O)N(R^{A3})—, —N(R^{A3})C(NR^{A3})N(R^{A3})—, —SO—, —S—, —SO2—, —S(O)₂N(R^{A3})-, or —N(R^{A3})SO₂-, where each R^{A3} is independently selected from hydrogen and methyl; and
Z¹ is a further oligonucleotide or is hydrogen, (1-6C)alkyl, (2-6C)alkenyl, (2-6C)alkynyl, aryl, (3-6C)cycloalkyl, (3-6C)cycloalkenyl, or heteroaryl,

where each (1 -6C)alkyl, (2-6C)alkenyl, (2-6C)alkynyl, aryl, (3-6C)cycloalkyl, (3-6C)cycloalkenyl, and heteroaryl is optionally substituted with one or more (e.g., 1, 2, 3, 4, or 5) substituent groups selected from the group consisting of (1-4C) alkyl, oxo, halo, cyano, nitro, hydroxy, carboxy, NR^{A4}R^{A5}, and (1-4C)alkoxy, where R^{A4} and R^{A5} are each independently selected from the group consisting of hydrogen and (1-4C)alkyl; and

each R² is independently -Y²-X²-Z², where
Y² is absent or a group of the formula —[CR^{B1}R^{B2}]ₘ- in which m is an integer selected from 1, 2, 3 or 4, and R^{B1} and R^{B2} are each independently selected from hydrogen, OH or (1-2C)alkyl;
X² is absent, —O—, —C(O)—, —C(O)O—, —OC(O)—, —CH(OR^{B3})—, —N(R^{B3})—, —N(R^{B3})—C(O)—, —N(R^{B3})—C(O)O—, —C(O)—N(R^{B3})—, —N(R^{B3})C(O)N(R^{B3})—, —N(R^{B3})C(NR^{B3})N(R^{B3})—, —SO—, —S— —SO₂—, —S(O)₂N(R^{B3})-, or —N(R^{B3})SO₂-, where each R^{B3} is independently selected from hydrogen or methyl; and
Z² is selected from hydrogen, (1-6C)alkyl, (2-6C)alkenyl, (2-6C)alkynyl, aryl, (3-6C)cycloalkyl, (3-6C)cycloalkenyl or heteroaryl, where each (1 -6C)alkyl, (2-6C)alkenyl, (2-6C)alkynyl, aryl, (3-6C)cycloalkyl, (3-6C)cycloalkenyl or heteroaryl is optionally substituted by one or more (e.g., 1, 2, 3, 4, or 5) substituent groups selected from the group consisting of (1-4C) alkyl, oxo, halo, cyano, nitro, hydroxy, carboxy, NR^{B4}R^{B5}, and (1-4C)alkoxy, where R^{B4} and R^{B5} are each independently hydrogen or (1-2C)alkyl; with the proviso that; when n=1 and T₁ and T₂ are different to one another, then R¹ and R² are not both H; when n=1, T₁ and T₂ are different to one another and one of R¹ and R² is H then the other of R¹ and R² is not methyl; or when n=2 and each occurrence of R¹ and R² is H, then T₁ and T₂ are both —C(O)- or are both —NH-.

In some embodiments, T₂ is —C(O)-.

In some embodiments, each R¹ is independently -Y¹-X¹-Z¹, where:

Y¹ is absent or —(CR^{A1}R^{A2})ₘ-, where m is 1, 2, 3 or 4, and R^{A1} and R^{A2} are each hydrogen or (1-2C)alkyl;

X¹ is absent, —O—, —C(O)—, —C(O)O—, —N(R^{A3})—, —N(R^{A3})—C(O)—, —C(O)—N(R^{A3})—, —N(R^{A3})C(O)N(R^{A3})—, —N(R^{A3})C(NR^{A3})N(R^{A3})- or —S-, where each R^{A3} is independently hydrogen or methyl; and

Z¹ is a further oligonucleotide or is hydrogen, (1-6C)alkyl, (2-6C)alkenyl, (2-6C)alkynyl, aryl, (3-6C)cycloalkyl, (3-6C)cycloalkenyl, or heteroaryl, where each (1-6C)alkyl, (2-6C)alkenyl, (2-6C)alkynyl, aryl, (3-6C)cycloalkyl, (3-6C)cycloalkenyl, and heteroaryl is optionally substituted by one or more (e.g., 1, 2, 3, 4, or 5) substituent groups selected from the group consisting of (1-4C) alkyl, oxo, halo, cyano, nitro, hydroxy, carboxy, NR^{A4}R^{A5}, and (1-4C)alkoxy, where R^{M} and R^{A5} are each independently hydrogen or (1-2C)alkyl.

In some embodiments, each R¹ is independently -Y¹-X¹-Z¹, where:

Y¹ is absent or —(CR^{A1}R^{A2})ₘ-, where m is 1, 2, 3, or 4, and R^{A1} and R″ are each independently hydrogen or (1-2C)alkyl;

X¹ is absent, —O—, —C (O)—, —C(O)O—, —N (R^{A3})—, —N(R^{A3})—C(O)—, —C (O)—N (R^{A3})—, —N(R^{A3})C(O)N(R^{A3})—, —N(R^{A3})C(NR^{A3})N(R^{A3})-, or —S-, where each R^{A3} is independently hydrogen or methyl; and

Z¹ is a further oligonucleotide or is hydrogen, (1-6C)alkyl, aryl, (3-6C)cycloalkyl, or heteroaryl, where each (1-6C)alkyl, aryl, (3-6C)cycloalkyl, and heteroaryl is optionally substituted by one or more (e.g., 1, 2, 3, 4, or 5) substituent groups selected from the group consisting of (1-4C) alkyl, halo, and hydroxy.

In some embodiments, each R¹ is independently -Y¹-X¹-Z¹, where:

Y¹ is absent or a group of the formula —(CR^{A1}R^{A2})ₘ-, where m is 1, 2, 3 or 4, and R^{A1} and R^{A2} are each independently hydrogen or (1-2C)alkyl;

X¹ is absent, —C(O)—, —C(O)O—, —N(R^{A3})—C(O)—, —C(O)—N(R^{A3})-, where each R^{A3} is hydrogen or methyl; and

Z¹ is a further oligonucleotide or is hydrogen, (1-6C)alkyl, aryl, (3-6C)cycloalkyl, or heteroaryl, where each (1-6C)alkyl, aryl, (3-6C)cycloalkyl, and heteroaryl is optionally substituted by one or more (e.g., 1, 2, 3, 4, or 5) substituent groups selected from the group consisting of (1-4C) alkyl, halo, and hydroxy.

In some embodiments, each R¹ is independently -Y¹-X¹-Z¹, where:

Y¹ is absent, —(CH₂)-, or —(CH₂CH₂)-;

X¹ is absent, —N(R^{A3})—C(O)—, —C(O)—N(R^{A3})-, where each R^{A3} is independently hydrogen or methyl; and

Z¹ is hydrogen or (1-2C)alkyl.

In some embodiments, each R² is independently -Y²-Z²,

where Y² is absent or —(CR^{B1} R^{B2})ₘ-, where m is 1, 2, 3 or 4, and R^{B1} and R^{B2} are each independently hydrogen or (1-2C)alkyl; and

Z² is hydrogen or (1-6C)alkyl.

In some embodiments, each R² is hydrogen. In some embodiments, n is 2 or 3. In some embodiments, n is 1.

In some embodiments, the linker is an amino acid residue selected from the group consisting of glutamic acid, succinic acid, and gamma-aminobutyric acid residues.

In some embodiments, the linker is of the following structure:

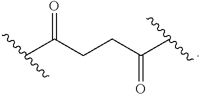 [SMILES: *C(=O)CCC(*)=O]

In some embodiments, the linker is of the following structure:

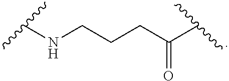 [SMILES: *NCCCC(*)=O]

In some embodiments, the linker is of the following structure:

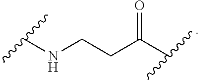 [SMILES: *NCCC(*)=O]

In some embodiments, the linker is of the following structure:

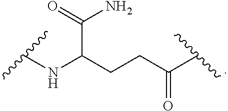 [SMILES: *NC(CCC(*)=O)C(N)=O]

In some embodiments, the linker is of the following structure:

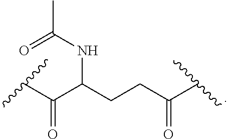 [SMILES: *C(=O)CCC(NC(C)=O)C(*)=O]

In some embodiments, the conjugate is of the following structure:

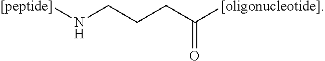

In some embodiments, the conjugate is of the following structure:

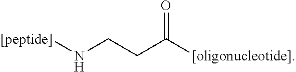

In some embodiments, the conjugate is of the following structure:

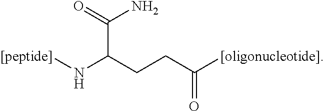

In some embodiments, the conjugate is of the following structure:

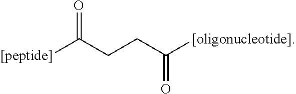

In some embodiments, the conjugate is of the following structure:

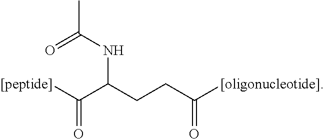

In some embodiments, the oligonucleotide is bonded to the linker or the peptide at its 3′ terminus.

In some embodiments, the conjugate is of the following structure:

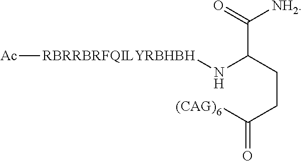

In some embodiments, the conjugate is of the following structure:

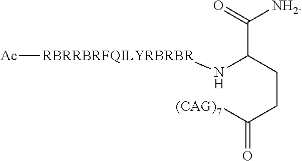

In some embodiments, the conjugate is of the following structure:

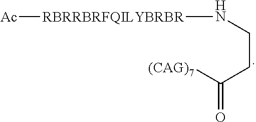

In some embodiments, the conjugate is of the following structure:

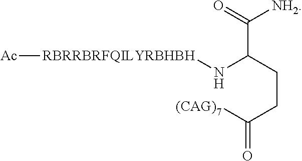

In some embodiments, the conjugate is of the following structure:

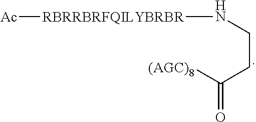

In some embodiments, the conjugate is of the following structure:

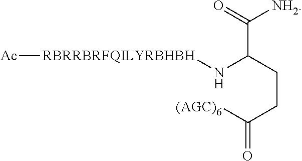

In some embodiments, the oligonucleotide is a morpholino. In some embodiments, all morpholino internucleoside linkages in the morpholino are —P(O)(NMe₂)O-. In some embodiments therefore the oligonucleotides is a phosphorodiamidate morpholino (PMO). In some embodiments, the oligonucleotide includes the following group as its 5′ terminus:

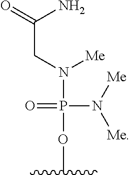 [SMILES: *OP(=O)(N(C)C)N(C)CC(N)=O]

In some embodiments, the conjugate is administered parenterally. In some embodiments, the conjugate is administered intravenously (e.g., by intravenous infusion).

In some embodiments, each dose within the plurality of doses includes at least 5 mg/kg (e.g., 5 mg/kg to 60 mg/kg, e.g., 30 mg/kg to 60 mg/kg; e.g., 5 mg/kg, 10 mg/kg, 20 mg/kg, 30 mg/kg, 40 mg/kg, 50 mg/kg, or 60 mg/kg, and ranges between any combination of any of these values) of the conjugate.

In some embodiments, each dose within the plurality of doses includes 40 mg/kg to 60 mg/kg, 30 mg/kg to 50 mg/kg, 30 mg/kg to 40 mg/kg, 40 mg/kg to 50 mg/kg, 50 mg/kg to 60 mg/kg, 35 mg/kg to 45 mg/kg, 45 mg/kg to 55 mg/kg, 35 mg/kg to 55 mg/kg, 30 mg/kg to 45 mg/kg, 35 mg/kg to 50 mg/kg, 40 mg/kg to 55 mg/kg, 45 mg/kg to 60 mg/kg, 1 mg/kg to 30 mg/kg, 1 mg/kg to 20 mg/kg, 5 mg/kg to 25 mg/kg, 10 mg/kg to 30 mg/kg, 1 mg/kg to 15 mg/kg, 5 mg/kg to 20 mg/kg, 10 mg/kg to 25 mg/kg, 15 mg/kg to 30 mg/kg, 1 mg/kg to 10 mg/kg, 5 mg/kg to 15 mg/kg, 10 mg/kg to 20 mg/kg, 15 mg/kg to 25 mg/kg, 20 mg/kg to 30 mg/kg, 1 mg/kg to 25 mg/kg, 4 mg/kg to 20 mg/kg, 6 mg/kg to 15 mg/kg, or 8 mg/kg to 10 mg/kg of the conjugate.

In some embodiments, each dose within the plurality of doses includes 1 mg/kg, 4 mg/kg, 5 mg/kg, 6 mg/kg, 8 mg/kg, 10 mg/kg, 15 mg/kg, 20 mg/kg, 25 mg/kg, 30 mg/kg, 35 mg/kg, 40 mg/kg, 45 mg/kg, 50 mg/kg, or 60 mg/kg of the conjugate.

The invention also includes the use of the conjugates described herein in the methods described herein. Accordingly, each method of treatment claim herein can be considered as supporting a claim in the form of a composition as specified therein for use in the indicated method (e.g., the treatment, prevention, or amelioration of DM1).

### Definitions

References to “X” throughout denote any form of the amino acid aminohexanoic acid, such as 6-aminohexanoic acid.

References to “B” throughout denote the amino acid beta-alanine.

Refences to “[Hyp]” throughout denote the amino acid hydroxyproline.

References to “Ac” throughout denote an acetyl group (CH₃—C(O)-).

References to other capital letters throughout denote the relevant genetically encoded amino acid residue in accordance with the accepted alphabetic amino acid code.

The term “alkyl,” as used herein, refers to a straight or branched chain hydrocarbon group containing a total of one to twenty carbon atoms, unless otherwise specified (e.g., (1-6C) alkyl, (1-4C) alkyl, (1-3C) alkyl, or (1-2C) alkyl). Non-limiting examples of alkyls include methyl, ethyl, 1-methylethyl, propyl, 1-methylbutyl, 1-ethylbutyl, etc. References to individual alkyl groups such as “propyl” are specific for the straight chain version only, and references to individual branched chain alkyl groups such as “isopropyl” are specific for the branched chain version only.

The term “alkenyl”, as used herein, refers to an aliphatic group containing having one, two, or three carbon-carbon double bonds and containing a total of two to twenty carbon atoms, unless otherwise specified (e.g., (2-6C) alkenyl, (2-4C) alkenyl, or (2-3C) alkenyl). Non-limiting examples of alkenyl include vinyl, allyl, homoallyl, isoprenyl, etc. Unless otherwise specified, alkenyl may be optionally substituted by one, two, three, four, or five groups selected from the group consisting of carbocyclyl, aryl, heterocyclyl, heteroaryl, oxo, halogen, and hydroxyl.

The term “alkynyl”, as used herein, refers to an aliphatic group containing one, two, or three carbon-carbon triple bonds and containing a total of two to twenty carbon atoms, unless otherwise specified (e.g., (2-6C) alkynyl, (2-4C) alkynyl, or (2-3C) alkynyl). Non-limiting examples of alkynyl include ethynyl, propargyl, homopropargyl, but-2-yn-1-yl, 2-methyl-prop-2-yn-1-yl, etc. Unless otherwise specified, alkynyl may be optionally substituted by one, two, three, four, or five groups selected from the group consisting of carbocyclyl, aryl, heterocyclyl, heteroaryl, oxo, halogen, and hydroxyl.

By “arginine rich” with respect to a cationic domain is meant that at least 40% of the cationic domain is formed of arginine residues.

The term “artificial amino acid,” as used herein, refers to an abiogenic amino acid (e.g., non-proteinogenic). For example, artificial amino acids may include synthetic amino acids, modified amino acids (e.g., those modified with sugars), non-natural amino acids, man-made amino acids, spacers, and non-peptide bonded spacers. Synthetic amino acids may be those that are chemically synthesized by man. For the avoidance of doubt, aminohexanoic acid (X) is an artificial amino acid in the context of the present invention. For the avoidance of doubt, beta-alanine (B) and hydroxyproline (Hyp) occur in nature and therefore are not artificial amino acids in the context of the present invention but are natural amino acids. Artificial amino acids may include, for example, 6-aminohexanoic acid (X), tetrahydroisoquinoline-3-carboxylic acid (TIC), 1-(amino)cyclohexanecarboxylic acid (Cy), 3-azetidine-carboxylic acid (Az), and 11-aminoundecanoic acid.

The term “aryl,” as used herein, refers to a carbocyclic ring system containing one, two, or three rings, at least one of which is aromatic. An unsubstituted aryl contains a total of 6 to 14 carbon atoms. The term aryl includes both monovalent species and divalent species. Examples of aryl groups include, but are not limited to, phenyl, naphthyl, indanyl, and the like. In particular embodiments, an optionally substituted aryl is optionally substituted phenyl.

By “bridged ring systems,” as used herein, are meant ring systems in which two rings share more than two atoms, see for example Advanced Organic Chemistry, by Jerry March, 4th Edition, Wiley Interscience, pages 131 -133, 1992. Examples of bridged heterocyclyl ring systems include, aza-bicydo[2.2.1]heptane, 2-oxa-5-azabicyclo[2.2.1]heptane, aza-bicyclo[2.2.2]octane, aza-bicyclo[3.2.1]octane, quinuclidine, etc.

The term “carbonyl,” as used herein, refers to a group of the following structure —C(O)-. Non-limiting examples of carbonyl groups include those found, e.g., in acetone, ethyl acetate, proteinogenic amino acids, acetamide, etc.

References made herein to “cationic” denote an amino acid or domain of amino acids having an overall positive charge at physiological pH.

The term “(m-nC)” or “(m-nC) group” used alone or as a prefix, refers to a group having a total of m to n carbon atoms, when unsubstituted.

The term “complementary,” as used herein in reference to a nucleobase sequence, refers to the nucleobase sequence having a pattern of contiguous nucleobases that permits an oligonucleotide having the nucleobase sequence to hybridize to another oligonucleotide or nucleic acid to form a duplex structure under physiological conditions. Complementary sequences include Watson-Crick base pairs formed from natural and/or modified nucleobases. Complementary sequences can also include non-Watson-Crick base pairs, such as wobble base pairs (guanosine-uracil, hypoxanthine-uracil, hypoxanthine-adenine, and hypoxanthine-cytosine) and Hoogsteen base pairs.

The term “cycloalkyl,” as used herein, refers to a saturated carbocyclic ring system containing one or two rings, and containing a total of 3 to 10 carbon atoms, unless otherwise specified. The two-ring cycloalkyls may be arranged as fused ring systems (two bridgehead carbon atoms are directly bonded to one another), bridged ring systems (two bridgehead carbon atoms are linked to one another via a covalent linker containing at least one carbon atom), and spiro-ring (two rings are fused at the same cabron atom) systems. Non-limiting examples of cycloalkyl include cyclopropyl, cyclobutyl, cyclopentyl, cyclohexyl, cycloheptyl, bicyclo[2.2.1]heptyl, etc.

The term “cycloalkenyl,” as used herein, refers to a non-aromatic, unsaturated, carbocyclic ring system containing one or two rings; containing one, two, or three endocyclic double bonds; and containing a total of 3 to 10 carbon atoms, unless otherwise specified. The two-ring cycloalkenyls may be arranged as fused ring systems (two bridgehead carbon atoms are directly bonded to one another), bridged ring systems (two bridgehead carbon atoms are linked to one another via a covalent linker containing at least one carbon atom), and spiro-ring (two rings are fused at the same cabron atom) systems. Non-limiting examples of cycloalkenyl include cyclobutenyl, cyclopentenyl, cyclohexenyl, cycloheptenyl, 3-cyclohexen-1-yl, cyclooctenyl, etc.

The term “halo” or “halogeno,” as used herein, refer to fluoro, chloro, bromo, and iodo.

By “histidine rich” with respect to a cationic domain it is meant that at least 40% of the cationic domain is formed of histidine residues.

The terms “heteroaryl” or “heteroaromatic,” as used interchangeably herein, refer to a ring system containing one, two, or three rings, at least one of which is aromatic and containing one to four (e.g., one, two, or three) heteroatoms selected from the group consisting of nitrogen, oxygen, and sulfur. An unsubstituted heteroaryl group contains a total of one to nine carbon atoms. The term heteroaryl includes both monovalent species and divalent species. Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example, a bicyclic structure formed from fused five and six membered rings or two fused six membered rings. Each ring may contain up to about four heteroatoms typically selected from nitrogen, sulfur and oxygen. Typically, the heteroaryl ring will contain up to 3 heteroatoms, more usually up to 2, for example, a single heteroatom. In some embodiments, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general, the number of basic nitrogen atoms present in the heteroaryl group, including any amino group substituents of the ring, will be less than five.

Examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3-b]furanyl, 2H-furo[3,2-b]-pyranyl, 5H-pyrido[2,3-d]-o-oxazinyl, 1 H-pyrazolo[4,3-d]-oxazolyl, 4H-imidazo[4,5-d]thiazolyl, pyrazino[2,3-d]pyridazinyl, imidazo[2,1-b]thiazolyl, imidazo[1,2-b][1,2,4]triazinyl. “Heteroaryl” also covers partially aromatic bi- or polycyclic ring systems where at least one ring is an aromatic ring and one or more of the other ring(s) is a non-aromatic, saturated or partially saturated ring, provided at least one ring contains one or more heteroatoms selected from nitrogen, oxygen or sulfur. Examples of partially aromatic heteroaryl groups include for example, tetrahydroisoquinolinyl, tetrahydroquinolinyl, 2-oxo-1.2.3.4-tetrahydroquinolinyl, dihydrobenzthienyl, dihydrobenzfuranyl, 2,3-dihydro-benzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl,1.2.3.4-tetrahydropyrido[2,3-b]pyrazinyl and 3,4-dihydro-2W-pyrido[3,2-b][1,4]oxazinyl. Examples of five membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups. Examples of six membered heteroaryl groups include but are not limited to pyridyl, pyrazinyl, pyridazinyl, pyrimidinyl and triazinyl. A bicyclic heteroaryl group may be, for example, a group selected from: a benzene ring fused to a 5- or 6-membered ring containing 1, 2 or 3 ring heteroatoms; a pyridine ring fused to a 5- or 6-membered ring containing 1, 2 or 3 ring heteroatoms; a pyrimidine ring fused to a 5- or 6-membered ring containing 1 or 2 ring heteroatoms; a pyrrole ring fused to a 5- or 6-membered ring containing 1, 2 or 3 ring heteroatoms; a pyrazole ring fused to a 5- or 6-membered ring containing 1 or 2 ring heteroatoms; a pyrazine ring fused to a 5- or 6-membered ring containing 1 or 2 ring heteroatoms; an imidazole ring fused to a 5- or 6-membered ring containing 1 or 2 ring heteroatoms; an oxazole ring fused to a 5- or 6-membered ring containing 1 or 2 ring heteroatoms; an isoxazole ring fused to a 5- or 6-membered ring containing 1 or 2 ring heteroatoms; a thiazole ring fused to a 5- or 6-membered ring containing 1 or 2 ring heteroatoms; an isothiazole ring fused to a 5- or 6-membered ring containing 1 or 2 ring heteroatoms; a thiophene ring fused to a 5- or 6-membered ring containing 1, 2 or 3 ring heteroatoms; a furan ring fused to a 5- or 6-membered ring containing 1, 2 or 3 ring heteroatoms; a cyclohexyl ring fused to a 5- or 6-membered heteroaromatic ring containing 1, 2 or 3 ring heteroatoms; and a cyclopentyl ring fused to a 5- or 6-membered heteroaromatic ring containing 1, 2 or 3 ring heteroatoms. Particular examples of bicyclic heteroaryl groups containing a six membered ring fused to a five membered ring include but are not limited to benzofuranyl, benzothiophenyl, benzimidazolyl, benzoxazolyl, benzisoxazolyl, benzothiazolyl, benzisothiazolyl, isobenzofuranyl, indolyl, isoindolyl, indolizinyl, indolinyl, isoindolinyl, purinyl (e.g., adeninyl, guaninyl), indazolyl, benzodioxolyl and pyrazolopyridinyl groups. Particular examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

The terms “heterocyclyl,” as used herein, refer to a ring system containing one, two, or three rings, at least one of which containing one to four (e.g., one, two, or three) heteroatoms selected from the group consisting of nitrogen, oxygen, and sulfur, provided that the ring system does not contain aromatic rings that also include an endocyclic heteroatom. An unsubstituted heterocyclyl group contains a total of two to nine carbon atoms. The term heterocyclyl includes both monovalent species and divalent species. Examples of heterocyclyl groups are monocyclic and bicyclic groups containing from five to twelve ring members, and more usually from five to ten ring members. The heterocyclyl group can be, for example, a 5- or 6-membered monocyclic ring or a 9- or 10-membered bicyclic ring, for example, a bicyclic structure formed from fused five and six membered rings or two fused six membered rings. Each ring may contain up to about four heteroatoms typically selected from nitrogen, sulfur and oxygen. Non-limiting examples of heterocyclyl groups include, e.g., pyrrolidine, piperazine, piperidine, azepane, 1,4-diazepane, tetrahydrofuran, tetrahydropyran, oxepane, 1,4-dioxepane, tetrahydrothiophene, tetrahydrothiopyran, indoline, benzopyrrolidine, 2,3-dihydrobenzofuran, phthalan, isochroman, and 2,3-dihydrobenzothiophene.

The term “internucleoside linkage,” as used herein, represents a group or bond that forms a covalent linkage between adjacent nucleosides in an oligonucleotide. An internucleoside linkage is an unmodified internucleoside linkage or a modified internucleoside linkage. An “unmodified internucleoside linkage” is a phosphate (—O—P(O)(OH)—O-) internucleoside linkage (“phosphate phosphodiester”). A “modified internucleoside linkage” is an internucleoside linkage other than a phosphate phosphodiester. The two main classes of modified internucleoside linkages are defined by the presence or absence of a phosphorus atom. Non-limiting examples of phosphorus-containing internucleoside linkages include phosphodiester linkages, phosphotriester linkages, phosphorothioate diester linkages, phosphorothioate triester linkages, morpholino internucleoside linkages, methylphosphonates, and phosphoramidate. Non-limiting examples of non-phosphorus internucleoside linkages include methylenemethylimino (—CH₂—N(CH₃)—O—CH₂—), thiodiester (—O—C(O)—S—), thionocarbamate (—O—C(O)(NH)—S—), siloxane (—O—Si(H)₂—O—), and N,N′-dimethylhydrazine (—CH₂—N(CH₃)—N(CH₃)—). Phosphorothioate linkages are phosphodiester linkages and phosphotriester linkages in which one of the non-bridging oxygen atoms is replaced with a sulfur atom. In some embodiments, an internucleoside linkage is a group of the following structure:

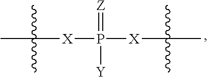

where

Z is O, S, or Se;

Y is —X—L—R¹;

each X is independently —O—, —S—, —N(—L—R¹)-, or L;

each L is independently a covalent bond or a linker (e.g., optionally substituted C₁₋₆₀ aliphatic linker or optionally substituted C₂₋₆₀ heteroaliphatic linker);

each R¹ is independently hydrogen, —S—S—R², —O—CO—R², —S—CO—R², optionally substituted C₁₋₉ heterocyclyl, or a hydrophobic moiety; and

each R² is independently optionally substituted C₁₋₁₀ alkyl, optionally substituted C₂₋₁₀ heteroalkyl, optionally substituted C₆₋₁₀ aryl, optionally substituted C₆₋₁₀ aryl C₁₋₆ alkyl, optionally substituted C₁₋₉ heterocyclyl, or optionally substituted C₁₋₉ heterocyclyl C₁₋₆ alkyl. When L is a covalent bond, R¹ is hydrogen, Z is oxygen, and all X groups are —O-, the internucleoside group is known as a phosphate phosphodiester. When L is a covalent bond, R¹ is hydrogen, Z is sulfur, and all X groups are —O-, the internucleoside group is known as a phosphorothioate diester. When Z is oxygen, all X groups are —O-, and either (1) L is a linker or (2) R¹ is not a hydrogen, the internucleoside group is known as a phosphotriester. When Z is sulfur, all X groups are —O-, and either (1) L is a linker or (2) R¹ is not a hydrogen, the internucleoside group is known as a phosphorothioate triester. Non-limiting examples of phosphorothioate triester linkages and phosphotriester linkages are described in US 2017/0037399, the disclosure of which is incorporated herein by reference.

An “intron” refers to a nucleic acid region (within a gene) that is not translated into a protein. An intron is a non-coding section that is transcribed into a precursor mRNA (pre-mRNA), and subsequently removed by splicing during formation of the mature RNA.

The term “morpholino,” as used herein in reference to a class of oligonucleotides, represents an oligomer of at least 10 morpholino monomer units interconnected by morpholino internucleoside linkages. A morpholino includes a 5′ group and a 3′ group. For example, a morpholino may be of the following structure:

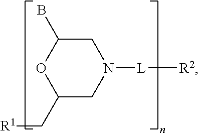

where

n is an integer of at least 10 (e.g., 12 to 30) indicating the number of morpholino subunits and associated groups L;

each B is independently a nucleobase;

R¹ is a 5′ group (R¹ may be referred to herein as a 5′ terminus);

R² is a 3′ group (R² may be referred to herein as a 3′ terminus); and

L is (i) a morpholino internucleoside linkage or, (ii) if L is attached to R², a covalent bond. A 5′ group in morpholino may be, e.g., hydroxyl, a hydrophobic moiety, phosphate, diphosphate, triphosphate, phosphorothioate, diphosphorothioate, triphosphorothioate, phosphorodithioate, disphorodithioate, triphosphorodithioate, phosphonate, phosphoramidate, a bond to a peptide, a bond to a peptide/linker combination, an endosomal escape moiety, or a neutral organic polymer. In some embodiments, the 5′ group is of the following structure:

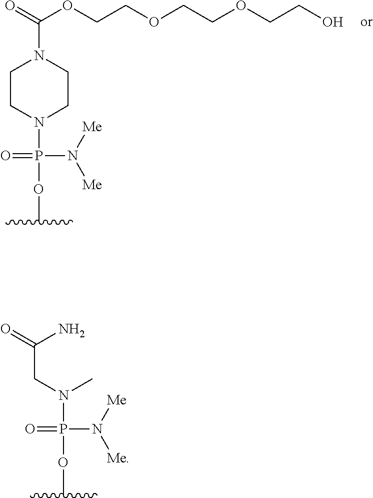 [SMILES: *OP(=O)(N(C)C)N1CCN(C(=O)OCCOCCOCCO)CC1 ; *OP(=O)(N(C)C)N(C)CC(N)=O]

Preferred 5′ group are hydroxyl and groups of the following structure:

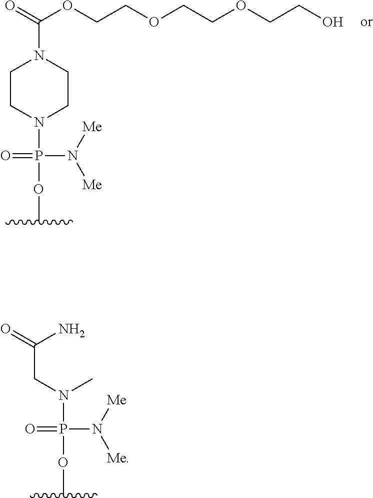 [SMILES: *OP(=O)(N(C)C)N1CCN(C(=O)OCCOCCOCCO)CC1 ; *OP(=O)(N(C)C)N(C)CC(N)=O]

A more preferred 5′ group is of the following structure:

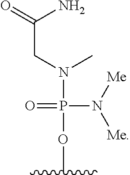 [SMILES: *OP(=O)(N(C)C)N(C)CC(N)=O]

A 3′ group in morpholino may be, e.g., hydrogen, a hydrophobic moiety, phosphate, diphosphate, triphosphate, phosphorothioate, diphosphorothioate, triphosphorothioate, phosphorodithioate, disphorodithioate, triphosphorodithioate, phosphonate, phosphoramidate, a bond to a peptide, a bond to a peptide/linker combination, an endosomal escape moiety, or a neutral organic polymer. In a conjugate of an oligonucleotide that is a morpholino and a peptide that is covalently bonded or linked to the oligonucleotide, the preferred 3′ group is a bond to a peptide or a bond to a peptide/linker combination.

The term “morpholino internucleoside linkage,” as used herein, represents a divalent group of the following structure:

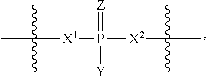

where

Z is O or S;

X¹ is a bond, —CH₂-, or —O-;

X² is a bond, —CH₂—O-, or —O-; and

Y is —NR₂, where each R is independently H or C₁₋₆ alkyl (e.g., methyl), or both R combine together with the nitrogen atom to which they are attached to form a C₂₋₉ heterocyclyl (e.g., N-piperazinyl); provided that both X¹ and X² are not simultaneously a bond.

The term “morpholino subunit,” as used herein, refers to the following structure:

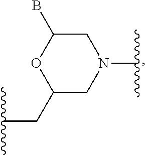

where B is a nucleobase.

The term “nucleobase,” as used herein, represents a nitrogen-containing heterocyclic ring found at the 1′ position of the ribofuranose/2′-deoxyribofuranose of a nucleoside. Nucleobases are unmodified or modified. As used herein, “unmodified” or “natural” nucleobases include the purine bases adenine (A) and guanine (G), and the pyrimidine bases thymine (T), cytosine (C), and uracil (U). Modified nucleobases include 5-substituted pyrimidines, 6-azapyrimidines, alkyl or alkynyl substituted pyrimidines, alkyl substituted purines, and N-2, N-6 and O-6 substituted purines, as well as synthetic and natural nucleobases, e.g., 5-methylcytosine, 5-hydroxymethyl cytosine, xanthine, hypoxanthine, 2-aminoadenine, 6-alkyl (e.g., 6-methyl) adenine and guanine, 2-alkyl (e.g., 2-propyl) adenine and guanine, 2-thiouracil, 2-thiothymine, 2-thiocytosine, 5-halouracil, 5-halocytosine, 5-propynyl uracil, 5-propynyl cytosine, 5-trifluoromethyl uracil, 5-trifluoromethyl cytosine, 7-methyl guanine, 7-methyl adenine, 8-azaguanine, 8-azaadenine, 7-deazaguanine, 7-deazaadenine, 3-deazaguanine, 3-deazaadenine. Certain nucleobases are particularly useful for increasing the binding affinity of nucleic acids, e g., 5-substituted pyrimidines; 6-azapyrimidines; N2-, N6-, and/or O6-substituted purines. Nucleic acid duplex stability can be enhanced using, e.g., 5-methylcytosine. Non-limiting examples of nucleobases include: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH3) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example, 7-deazaadenine, 7-deazaguanine, 2-aminopyridine, or 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

The term “nucleoside,” as used herein, represents sugar-nucleobase compounds and groups known in the art, as well as modified or unmodified 2′-deoxyribofuranrpose-nucleobase compounds and groups known in the art. The sugar may be ribofuranose. The sugar may be modified or unmodified. An unmodified ribofuranose-nucleobase is ribofuranose having an anomeric carbon bond to an unmodified nucleobase. Unmodified ribofuranose-nucleobases are adenosine, cytidine, guanosine, and uridine. Unmodified 2′-deoxyribofuranose-nucleobase compounds are 2′-deoxyadenosine, 2′-deoxycytidine, 2′-deoxyguanosine, and thymidine. The modified compounds and groups include one or more modifications selected from the group consisting of nucleobase modifications and sugar modifications described herein. A nucleobase modification is a replacement of an unmodified nucleobase with a modified nucleobase. A sugar modification may be, e.g., a 2′-substitution, locking, carbocyclization, or unlocking. A 2′-substitution is a replacement of 2′-hydroxyl in ribofuranose with 2′-fluoro, 2′-methoxy, or 2′-(2-methoxy)ethoxy. Alternatively, a 2′-substitution may be a 2′-(ara) substitution, which corresponds to the following structure:

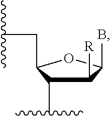

where B is a nucleobase, and R is a 2′-(ara) substituent (e.g., fluoro). 2′-(ara) substituents are known in the art and can be same as other 2′-substituents described herein. In some embodiments, 2′-(ara) substituent is a 2′-(ara)-F substituent (R is fluoro). A locking modification is an incorporation of a bridge between 4′-carbon atom and 2′-carbon atom of ribofuranose. Nucleosides having a locking modification are known in the art as bridged nucleic acids, e.g., locked nucleic acids (LNA), ethylene-bridged nucleic acids (ENA), and cEt nucleic acids. The bridged nucleic acids are typically used as affinity enhancing nucleosides. A “nucleoside” may also refer to a morpholino subunit.

The term “nucleotide,” as used herein, represents a nucleoside bonded to an internucleoside linkage or a monovalent group of the following structure -X¹-P(X²)(R¹)₂, where X¹ is O, S, or NH, and X² is absent, ═O, or ═S, and each R¹ is independently —OH, —N(R²)₂, or —O—CH₂CH₂CN, where each R² is independently an optionally substituted alkyl, or both R² groups, together with the nitrogen atom to which they are attached, combine to form an optionally substituted heterocyclyl.

The term “oligonucleotide,” as used herein, represents a structure containing 10 or more contiguous nucleosides covalently bound together by internucleoside linkages; a morpholino containing 10 or more morpholino subunits; or a peptide nucleic acid containing 10 or more morpholino subunits. Preferably, an oligonucleotide is a morpholino.

The term “optionally substituted” refers to groups, structures, or molecules that may be substituted or unsubstituted as described for each respective group. The term “where a/any CH, CH₂, CH₃ group or heteroatom (i.e., NH) within a R¹ group is optionally substituted” means that (any) one of the hydrogen radicals of the R¹ group is substituted by a relevant stipulated group.

In this specification the term “operably linked” may include the situation where a selected nucleotide sequence and regulatory nucleotide sequence are covalently linked in such a way as to place the expression of a nucleotide coding sequence under the control of the regulatory sequence, as such, the regulatory sequence is capable of effecting transcription of a nucleotide coding sequence which forms part or all of the selected nucleotide sequence. Where appropriate, the resulting transcript may then be translated into a desired peptide.

The term “pharmaceutically acceptable,” as used herein, refers to those compounds, materials, compositions, and/or dosage forms, which are suitable for contact with the tissues of an individual (e.g., a human), without excessive toxicity, irritation, allergic response and other problem complications commensurate with a reasonable benefit/risk ratio.

The term “pharmaceutical composition,” as used herein, represents a composition containing an oligonucleotide described herein, formulated with a pharmaceutically acceptable excipient, and manufactured or sold with the approval of a governmental regulatory agency as part of a therapeutic regimen for the treatment of disease in a subject.

The term “pharmaceutically acceptable salt,” as used herein, means any pharmaceutically acceptable salt of a conjugate, oligonucleotide, or peptide disclosed herein. Pharmaceutically acceptable salts of any of the compounds described herein may include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use (Eds. P. H. Stahl and C. G. Wermuth), Wiley-VCH, 2008. The salts can be prepared in situ during the final isolation and purification of the compounds described herein or separately by reacting a free base group with a suitable acid. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

The term “reduce” or “inhibit” may relate generally to the ability of one or more compounds of the invention to “decrease” a relevant physiological or cellular response, such as a symptom of a disease or condition described herein, as measured according to routine techniques in the diagnostic art. Relevant physiological or cellular responses (in vivo or in vitro) will be apparent to persons skilled in the art, and may include reductions in the symptoms or pathology of myotonic dystrophy type 1, or reductions in the expression of defective forms of DMPK gene, such as the altered forms of DMPK gene that are expressed in individuals with myotonic dystrophy 1. A “decrease” in a response may be statistically significant as compared to the response produced by no antisense compound or a control composition, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease, including all integers in between.

The term “subject,” as used herein, represents a human or non-human animal (e.g., a mammal) that is suffering from, or is at risk of, disease, disorder, or condition, as determined by a qualified professional (e.g., a doctor or a nurse practitioner) with or without known in the art laboratory test(s) of sample(s) from the subject. Non-limiting examples of diseases, disorders, and conditions include myotonic dystrophy type 1.

A “sugar” or “sugar moiety,” includes naturally occurring sugars having a furanose ring or a structure that is capable of replacing the furanose ring of a nucleoside. Sugars included in the nucleosides of the invention may be non-furanose (or 4′-substituted furanose) rings or ring systems or open systems. Such structures include simple changes relative to the natural furanose ring (e.g., a six-membered ring). Alternative sugars may also include sugar surrogates where the furanose ring has been replaced with another ring system such as, e.g., a morpholino or hexitol ring system. Non-limiting examples of sugar moieties useful that may be included in the oligonucleotides of the invention include β-D-ribose, β-D-2′-deoxyribose, substituted sugars (e.g., 2′, 5′, and bis substituted sugars), 4′-S-sugars (e.g., 4′-S-ribose, 4′-S-2′-deoxyribose, and 4′-S-2′-substituted ribose), bicyclic sugar moieties (e.g., the 2′-O—CH₂-4′ or 2′-O—(CH₂)₂-4′ bridged ribose derived bicyclic sugars) and sugar surrogates (when the ribose ring has been replaced with a morpholino or a hexitol ring system).

“Treatment” and “treating,” as used herein, refer to the medical management of a subject with the intent to improve, ameliorate, or stabilize a disease, disorder, or condition (e.g., myotonic dystrophy type 1). This term includes active treatment (treatment directed to improve myotonic dystrophy type 1); palliative treatment (treatment designed for the relief of symptoms of myotonic dystrophy type 1); and supportive treatment (treatment employed to supplement another therapy).

Throughout the description and claims of this specification, the words “include” and “contain” and variations of them mean “including but not limited to,” and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

All references to “conjugates” also refer to solvates thereof, including pharmaceutically acceptable solvates thereof.

All references to “oligonucleotides” also refer to salts and/or solvates thereof, including pharmaceutically acceptable salts and/or solvates thereof.

Unless otherwise specified, all peptides are shown herein in N-terminus to C-terminus direction (left to right). Unless otherwise specified, all oligonucleotides are shown herein in 5′ to 3′ direction (left to right).

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive.

### BRIEF DESCRIPTION OF THE DRAWINGS

FIG. 1: shows the structure of PPMO conjugate.

FIG. 2: shows in vivo correction of functional defects in the HSALR DM1 mouse model by single intravenous bolus administration of different dose levels of PPMO conjugate. Correction of myotonia (measured as the area under the force/time curve during relaxation after maximal muscle contraction) in gastrocnemius muscle from control (WT) and myotonic (HSALR) mice by electromyographic myotonia measurements 2 weeks after administration of PPMO conjugate. Data expressed with whiskers from min to max, the dashed line indicates where there is no myotonia at the myotonia null threshold. Graph plotted as mean±SEM, n=4-16 per group. Statistics were performed using the one-way ANOVA Dunnett's multiple comparison test, and the significant values shown are vs HSALR saline, **P<0.01, ***P<0.001.

FIG. 3: shows in vivo correction of molecular defects in the quadriceps and gastrocnemius muscles of the HSALR DM1 mouse model. Quantified splicing correction analysis by RT-PCR of Clcn1 transcripts, Mbnl1 transcripts, and Atp2a1 transcripts was carried out on muscle from control (WT) and myotonic (HSALR) mice 2 weeks after administration of PPMO conjugate with a single bolus administration at multiple dose levels. Data is represented as mean±SEM, n=4-16 per group. Statistics were performed using the one-way ANOVA Dunnett's multiple comparison test, and the significant values shown are vs HSALR saline, *P<0.05, **P<0.01, ***P<0.001, ****P<0.0001.

FIG. 4: shows the in vivo screening of PPMO in the HSALR DM1 mouse model for the levels of CUGexp HSA transcripts in gastrocnemius (FIG. 4a) and quadriceps (FIG. 4b) muscle as determined by qPCR. Data is represented as mean±SEM, n=4-16 per group. Statistics were performed using the one-way ANOVA Dunnett's multiple comparison test, and the significant values shown are vs HSALR saline, ****P<0.0001.

FIG. 5: shows changes in control human myoblast cell viability in vitro over 12, 24, 36, and 48 hours after transfection with increasing concentrations of PPMO and compared to myoblast cells transfected with unconjugated PMO or Pip-conjugated PMO (Pip-PMO). Graph plotted as mean±SEM, n per group. Statistics were performed using the one-way ANOVA Dunnett's multiple comparison test, and the significant values shown are vs PBS (NT), ***P<0.001).

FIG. 6: shows PMODmi targets CUG repeat and works through steric blocking. PPMO conjugate has no impact on nuclear foci numbers in gastrocnemius muscle. n≥8 per treatment group per parameter. Graph plotted as mean±SEM. Statistics were performed using the one-way ANOVA Dunnett's multiple comparison test, and the significant values shown are vs HSALR saline (not significant (ns)>0.05).

FIG. 7: shows PPMO conjugate off target assessment. Off target analysis performed to assess impact of a repeat sequence PMO on naturally occurring CUG repeats. PPMO conjugate has no significant effects on Mapkap1 or Pcolce whereas the level of TxInb transcript is moderately elevated compared to baseline. n=8 per treatment group per parameter. Graph plotted as mean±SEM. Statistics were performed using the one-way ANOVA Dunnett's multiple comparison test, and the significant values shown are vs HSALR saline (not significant (ns)>0.05, “”P<0.01, ***P<0.001, ****P<0.0001).

FIG. 8: shows that serum clinical chemistry levels are unchanged from saline ranges. Levels of urea, creatinine, creatine kinase, albumin, alkaline phosphatase (ALP), alanine transferase (ALT), and aspartate aminotransferase (AST) measured in serum of wild-type (WT) and HSALR mice (8-12 weeks old,) after administration of saline or PPMO conjugate at indicated doses by bolus IV (tail vein) administration, are shown. Serum was harvested for analysis 14 days post-administration. Graph plotted as mean±SEM, n=4-8 per group. Statistics were performed using the one-way ANOVA Dunnett's multiple comparison test, and the significant values shown are vs HSALR saline (not significant (ns)>0.05, *P<0.05).

FIG. 9: shows that tissue bioanalysis identifies PMO detection in key tissues. Dose response of PMO detected in skeletal muscle. LLOQ, lower limit of linear quantitation. n=4-8. Graph plotted as mean±SEM.

FIG. 10: shows that PPMO conjugate correction of pathogenic mis-splicing has an unchanged lasting effect in skeletal muscle. Single administration of PPMO conjugate can correct the mis-splicing molecular events in a DM1 mouse model for up to 12 weeks. NT, no treatment (0.9% saline control). n=7-8 per group. Graph plotted as mean±SEM.

FIG. 11: shows that PPMO conjugate correction of pathogenic mis-splicing has a lasting effect in a DM1 mouse model. Single administration of PPMO conjugate can correct the mis-splicing molecular events in a DM1 mouse model for up to 12 weeks. Treatment with PPMO conjugate does not impact splicing levels in wild type (WT) mice. NT, no treatment (0.9% saline control). n=7-8 per group. Graph plotted as mean±SD.

FIG. 12: shows that PPMO conjugate reduces the number of pathogenic nuclear foci, a hallmark of DM1, in immortalized myoblasts in a dose-dependent manner.

FIGS. 13A-13E: show that PPMO conjugate treatment and liberation of MBNL1 resulted in robust correction of downstream mis-splicing. Mean±SEM; n=3-4 per group. FIG. 13A shows percentage splice inclusion levels for MBNL1 exon 5 in healthy cells, as well as in DM1 patient cells treated with unconjugated PMO or PPMO conjugate. FIG. 13B shows percentage splice inclusion levels for MBNL2 exon 5 in healthy cells, as well as in DM1 patient cells treated with unconjugated PMO or PPMO conjugate. FIG. 13C shows percentage splice inclusion levels for BIN1 exon 7 in healthy cells, as well as in DM1 patient cells treated with unconjugated PMO or PPMO conjugate. FIG. 13D shows percentage splice inclusion levels for LDB3 exon 11 in healthy cells, as well as in DM1 patient cells treated with unconjugated PMO or PPMO conjugate. FIG. 13E shows percentage splice inclusion levels for SORBS1 exon 25 in healthy cells, as well as in DM1 patient cells treated with unconjugated PMO or PPMO conjugate.

FIGS. 14A and 14B: show Atp2a1 exon 22 inclusion levels and Clcn1 exon 7a inclusion levels, respectively. The inclusion levels were assessed in gastrocnemius (lower trace in FIG. 14A, upper trace in FIG. 14B) and quadriceps (upper trace in FIG. 14A, lower trace in FIG. 14B). Graph plotted as mean±SEM; n=7 for 0 timepoint; 8 for 2- and 12-week timepoints; 5 for 24-week timepoint. The results show that the conjugate sustained molecular correction of mis-splicing for at least 24 weeks following a single dose.

### DETAILED DESCRIPTION

In general, the invention provides methods of treating a subject having myotonic dystrophy type 1 (DM1). The methods include administering a therapeutic regimen including a plurality of doses of a conjugate spaced at a time interval of, e.g., at least 1 month (e.g., 1 to 6 months, 2 to 6 months, 3 to 6 months, 3 to 4 months, 4 to 6 months, 5 to 6 months; e.g., 1 month, 2 months, 3 months, 4 months, 5 months, or 6 months), where the conjugate includes an oligonucleotide and a peptide covalently bonded or linked via a linker to the oligonucleotide. The therapeutic regimen may further include a treatment initiation regimen including administering the conjugate three or four times at an initiation interval of 2 weeks.

Accordingly, in some embodiments, the time interval is 1 to 6 months. In some embodiments, the time interval is 2 to 6 months. In some embodiments, the time interval is 3 to 6 months. In some embodiments, the time interval is 4 to 6 months. In some embodiments, the time interval is 5 to 6 months. In some embodiments, the interval is 1 to 2 months. In some embodiments the interval is 1 to 3 months. In some embodiments the interval is 1 to 4 months. In some embodiments the interval is 1 to 5 months. In some embodiments the interval is 2 to 3 months. In some embodiments the interval is 2 to 4 months. In some embodiments the interval is 2 to 5 months. In some embodiments the interval is 3 to 4 months. In some embodiments the interval is 3 to 5 months. In some embodiments the interval is 4 to 5 months. In some embodiments, the time interval is 1 month, 2 months, 3 months, 4 months, 5 months, or 6 months. In some embodiments, the interval is 30 days, 45 days, 60 days, 75 days, 90 days, 105 days, or 120 days.

In some embodiments, the therapeutic regimen further includes administering a treatment initiation or loading regimen including administering the conjugate two, three, four, or five times at an initiation interval of 1, 2, or 3 weeks. In some embodiments, this initiation or loading regimen is followed by a maintenance regimen that can be selected, for example, from any one of the regimens listed in the prior paragraph.

In some embodiments, the amount of conjugate is administered at the same dose level each time.

In some embodiments, the dose is selected from the group consisting of a single dose per interval of: 5 mg/kg, 10 mg/kg, 15 mg/kg, 20 mg/kg, 25 mg/kg, 30 mg/kg, 40 mg/kg, 45 mg/kg, 50 mg/kg, 55 mg/kg, 60 mg/kg, or at an amount within a range between a selection of any combination of any of these values. Accordingly, in some embodiments, the single dose per interval can be, for example, 5-60 mg/kg, 5-50 mg/kg, 5-40 mg/kg, 5-30 mg/kg, 5-20 mg/kg, 5-10 mg/kg, 10-60 mg/kg, 10-50 mg/kg, 10-40 mg/kg, 10-30 mg/kg, 10-20 mg/kg, 20-60 mg/kg, 20-50 mg/kg, 20-40 mg/kg, 20-30 mg/kg, 30-60 mg/kg, 30-50 mg/kg, 30-40 mg/kg, 40-50 mg/kg, 40-60 mg/kg, or 50-60 mg/kg.

In some embodiments, the administration continues for at least 0.5, 1, 2, 3, 4, 5, 10, 15, 20, 25, 30, 35, or more years (e.g., for a patient's lifetime).

The peptide includes a hydrophobic domain flanked by two cationic domains, each of the cationic domains including one of RBRRBRR (SEQ ID NO: 1), RBRBR (SEQ ID NO: 2), RBRR (SEQ ID NO: 3), RBRRBR (SEQ ID NO: 4), RRBRBR (SEQ ID NO: 5), RBRRB (SEQ ID NO: 6), BRBR (SEQ ID NO: 7), RBHBH (SEQ ID NO: 8), HBHBR (SEQ ID NO: 9), RBRHBHR (SEQ ID NO: 10), RBRBBHR (SEQ ID NO: 11), RBRRBH (SEQ ID NO: 12), HBRRBR (SEQ ID NO: 13), HBHBH (SEQ ID NO: 14), BHBH (SEQ ID NO: 15), BRBSB (SEQ ID NO: 16), BRB[Hyp]B (SEQ ID NO: 17), R[Hyp]H[Hyp]HB (SEQ ID NO: 18), and R[Hyp]RR[Hyp]R (SEQ ID NO: 19), and the hydrophobic domain including one of YQFLI (SEQ ID NO: 20), FQILY (SEQ ID NO: 21), ILFQY (SEQ ID NO: 22), FQIY (SEQ ID NO: 23), WWW, WWPWW (SEQ ID NO: 24), WPWW (SEQ ID NO: 25), and WWPW (SEQ ID NO: 26). The oligonucleotide includes a total of 12 to 40 contiguous nucleobases, where at least 9 contiguous nucleobases are complementary to a CUG repeat sequence.

Advantageously, the methods described herein provide a therapeutically effective amount of the conjugate of the invention while reducing toxicological effects of the therapy. Furthermore, in providing surprisingly long-lasting effects, the methods of the invention provide advantages with respect to patient compliance with treatment, comfort, and convenience. Accordingly, the methods described and claimed herein represent substantial advances for the treatment of DM1.

### Oligonucleotides

Oligonucleotides used in the conjugates disclosed herein may be those complementary to the expanded CUG repeats within the 3′-untranslated region of dystrophia myotonica-protein kinase (DMPK) transcript. Without wishing to be bound by theory, it is believed that an oligonucleotide hybridizing to the expanded CUG repeats within the 3′-untranslated region of DMPK transcripts may reduce the incidence of the DMPK transcript missplicing, thereby ameliorating myotonic dystrophy type 1.

In some embodiments, the oligonucleotide is 5′-[CAG]ₙ-3′, where n is an integer from 5 to 8. In some embodiments, the oligonucleotide is 5′-[CAG]₅-3′. In some embodiments, the oligonucleotide is 5′-[CAG]₆-3′. In some embodiments, the oligonucleotide is 5′-[CAG]₇-3′. In some embodiments, the oligonucleotide is 5′-[CAG]₈-3′.

In some embodiments, the oligonucleotide is 5′-[AGC]ₙ-3′, where n is an integer from 5 to 8. In some embodiments, the oligonucleotide is 5′-[AGC]₅-3′. In some embodiments, the oligonucleotide is 5′-[AGC]₆-3′. In some embodiments, the oligonucleotide is 5′-[AGC]₇-3′. In some embodiments, the oligonucleotide is 5′-[AGC]₈-3′.

In some embodiments, the oligonucleotide is 5′-[GCA]ₙ-3′, where n is an integer from 5 to 8. In some embodiments, the oligonucleotide is 5′-[GCA]₅-3′. In some embodiments, the oligonucleotide is 5′-[GCA]₆-3′. In some embodiments, the oligonucleotide is 5′-[GCA]₇-3′. In some embodiments, the oligonucleotide is 5′-[GCA]₈-3′.

In some embodiments, the oligonucleotide is an oligonucleotide molecule as described herein. In some embodiments, the oligonucleotide is a phosphorodiamidate morpholino oligonucleotide (PMO) as described herein.

### Peptides

Peptides that may be used in the conjugates described herein include those disclosed in WO 2020030927 and WO 2020115494. In some embodiments, peptides included in the conjugates described herein include no artificial amino acid residues.

In some embodiments, the peptide does not contain aminohexanoic acid residues. In some embodiments, the peptide does not contain any form of aminohexanoic acid residues. In some embodiments, the peptide does not contain 6-aminohexanoic acid residues.

In some embodiments, the peptide contains only natural amino acid residues, and therefore consists of natural amino acid residues.

In some embodiments, artificial amino acids such as 6-aminohexanoic acid that are typically used in cell-penetrating peptides are replaced by natural amino acids. In some embodiments, the artificial amino acids such as 6-aminohexanoic acid that are typically used in cell-penetrating peptides are replaced by amino acids selected from beta-alanine, serine, proline, arginine, and histidine or hydroxyproline.

In some embodiments, aminohexanoic acid is replaced by beta-alanine. In some embodiments, 6-aminohexanoic acid is replaced by beta-alanine

In some embodiments, aminohexanoic acid is replaced by histidine. In some embodiments, 6-aminohexanoic acid is replaced by histidine.

In some embodiments, aminohexanoic acid is replaced by hydroxyproline. In some embodiments, 6-aminohexanoic acid is replaced by hydroxyproline.

In some embodiments, the artificial amino acids such as 6-aminohexanoic acid that are typically used in cell-penetrating peptides may be replaced by a combination of any of beta-alanine, serine, proline, arginine, and histidine or hydroxyproline, e.g., a combination of any of beta-alanine, histidine, and hydroxyproline.

In some embodiments, there is provided a peptide having a total length of 40 amino acid residues or less, the peptide including: two or more cationic domains each including at least 4 amino acid residues; and one or more hydrophobic domains each including at least 3 amino acid residues; where at least one cationic domain includes histidine residues. In some embodiments, where at least one cationic domain is histidine rich.

In some embodiments, what is meant by histidine rich is defined herein in relation to the cationic domains.

### Cationic Domain

The present invention relates to short cell-penetrating peptides having a particular structure in which there are at least two cationic domains having a certain length.

In some embodiments, the peptide includes up to 4 cationic domains, up to 3 cationic domains.

In some embodiments, the peptide includes 2 cationic domains.

As defined above, the peptide includes two or more cationic domains each having a length of at least 4 amino acid residues.

In some embodiments, each cationic domain has a length of between 4 to 12 amino acid residues, e.g., a length of between 4 to 7 amino acid residues.

In some embodiments, each cationic domain has a length of 4, 5, 6, or 7 amino acid residues.

In some embodiments, each cationic domain is of similar length, e.g., each cationic domain is the same length.

In some embodiments, each cationic domain includes cationic amino acids and may also contain polar and or nonpolar amino acids.

Non-polar amino acids may be selected from: alanine, beta-alanine, proline, glycine, cysteine, valine, leucine, isoleucine, methionine, tryptophan, phenylalanine. In some embodiments, non-polar amino acids do not have a charge.

Polar amino acids may be selected from: serine, asparagine, hydroxyproline, histidine, arginine, threonine, tyrosine, glutamine. In some embodiments, the selected polar amino acids do not have a negative charge.

Cationic amino acids may be selected from: arginine, histidine, lysine. In some embodiments, cationic amino acids have a positive charge at physiological pH.

In some embodiments, each cationic domain does not include anionic or negatively charged amino acid residues. In some embodiments, each cationic domain includes arginine, histidine, beta-alanine, hydroxyproline, and/or serine residues.

In some embodiments, each cationic domain consists of arginine, histidine, beta-alanine, hydroxyproline, and/or serine residues.

In some embodiments, each cationic domain includes at least 40%, at least 45%, at least 50% cationic amino acids.

In some embodiments, each cationic domain includes a majority of cationic amino acids. In some embodiments, each cationic domain includes at least 55%, at least 60%, at least 65% at least 70%, at least 75%, at least 80%, at least 85%, at least 90%, at least 95% cationic amino acids.

In some embodiments, each cationic domain includes an isoelectric point (pi) of at least 7.5, at least 8.0, at least 8.5, at least 9.0, at least 9.5, at least 10.0, at least 10.5, at least 11.0, at least 11.5, at least 12.0.

In some embodiments, each cationic domain includes an isoelectric point (pi) of at least 10.0.

In some embodiments, each cationic domain includes an isoelectric point (pi) of between 10.0 and 13.0

In some embodiments, each cationic domain includes an isoelectric point (pi) of between 10.4 and 12.5.

In some embodiments, the isoelectric point of a cationic domain is calculated at physiological pH by any suitable means available in the art. In some embodiments, by using the I PC (www.isoelectric.org) a web-based algorithm developed by Lukasz Kozlowski, Biol. Direct. 2016; 11:55. DOI: 10.1186/s 13062-016-0159-9.

In some embodiments, each cationic domain includes at least 1 cationic amino acid, e.g., 1-5 cationic amino acids. In some embodiments, each cationic domain includes at least 2 cationic amino acids, e.g., 2-5 cationic amino acids.

In some embodiments, each cationic domain is arginine rich and/or histidine rich. In some embodiments, a cationic domain may contain both histidine and arginine.

In some embodiments, each cationic domain includes a majority of arginine and/or histidine residues.

In some embodiments, each cationic domain includes at least 40%, at least 45%, at least 50%, at least 55%, at least 60%, at least 60%, at least 65%, or at least 70% arginine and/or histidine residues. In some embodiments, a cationic domain may include at least 40%, at least 45%, at least 50%, at least 55%, at least 60%, at least 60%, at least 65%, or at least 70% arginine residues.

In some embodiments, a cationic domain may include at least 40%, at least 45%, at least 50%, at least 55%, at least 60%, at least 60%, at least 65%, or at least 70% histidine residues.

In some embodiments, a cationic domain may include a total of between 1-5 histidine and 1-5 arginine residues. In some embodiments, a cationic domain may include between 1-5 arginine residues.

In some embodiments, a cationic domain may include between 1-5 histidine residues. In some embodiments, a cationic domain may include a total of between 2-5 histidine and 3-5 arginine residues.

In some embodiments, a cationic domain may include between 3-5 arginine residues. In some embodiments, a cationic domain may include between 2-5 histidine residues.

In some embodiments, each cationic domain includes one or more beta-alanine residues. In some embodiments, each cationic domain may include a total of between 2-5 beta-alanine residues, e.g., a total of 2 or 3 beta-alanine residues.

In some embodiments, a cationic domain may include one or more hydroxyproline residues or serine residues.

In some embodiments, a cationic domain may include between 1-2 hydroxyproline residues. In some embodiments, a cationic domain may include between 1-2 serine residues.

In some embodiments, all of the cationic amino acids in a given cationic domain may be histidine, alternatively, e.g., all of the cationic amino acids in a given cationic domain may be arginine.

In some embodiments, the peptide may include at least one histidine rich cationic domain. In some embodiments, the peptide may include at least one arginine rich cationic domain.

In some embodiments, the peptide may include at least one arginine rich cationic domain and at least one histidine rich cationic domain.

In some embodiments, the peptide includes two arginine rich cationic domains.

In some embodiments, the peptide includes two histidine rich cationic domains.

In some embodiments, the peptide includes two arginine and histidine rich cationic domains.

In some embodiments, the peptide includes one arginine rich cationic domain and one histidine rich cationic domain. In some embodiments, each cationic domain includes no more than 3 contiguous arginine residues, e.g., no more than 2 contiguous arginine residues.

In some embodiments, each cationic domain includes no contiguous histidine residues.

In some embodiments, each cationic domain includes arginine, histidine, and/or beta-alanine residues. In some embodiments, each cationic domain includes a majority of arginine, histidine, and/or beta-alanine residues. In some embodiments, at least 70%, at least 75%, at least 80%, at least 85%, at least 90%, at least 95%, or 100% of the amino acid residues in each cationic domain are arginine, histidine, and/or beta-alanine residues. In some embodiments, each cationic domain consists of arginine, histidine, and/or beta-alanine residues.

In some embodiments, the peptide includes a first cationic domain including arginine and beta-alanine residues and a second cationic domain including arginine and beta-alanine residues.

In some embodiments, the peptide includes a first cationic domain including arginine and beta-alanine resides, and a second cationic domain including histidine, beta-alanine, and optionally arginine residues.

In some embodiments, the peptide includes a first cationic domain including arginine and beta-alanine resides, and a second cationic domain including histidine and beta-alanine residues.

In some embodiments, the peptide includes a first cationic domain consisting of arginine and beta-alanine residues and a second cationic domain consisting of arginine and beta-alanine residues.

In some embodiments, the peptide includes a first cationic domain consisting of arginine and beta-alanine residues and a second cationic domain consisting of arginine, histidine, and beta-alanine residues.

In some embodiments, the peptide includes at least two cationic domains, e.g., these cationic domains form the arms of the peptide. In some embodiments, the cationic domains are located at the N and C terminus of the peptide. In some embodiments, therefore, the cationic domains may be known as the cationic arm domains.

In some embodiments, the peptide includes two cationic domains, where one is located at the N-terminus of the peptide and one is located at the C-terminus of the peptide. In some embodiments, at either end of the peptide. In some embodiments, no further amino acids or domains are present at the N-terminus and C-terminus of the peptide, with the exception of other groups such as a terminal modification, linker and/or oligonucleotide. For the avoidance of doubt, such other groups may be present in addition to ‘the peptide’ described and claimed herein. In some embodiments, therefore each cationic domain forms the terminus of the peptide. In some embodiments, this does not preclude the presence of a further linker group as described herein.

In some embodiments, the peptide may include up to 4 cationic domains. In some embodiments, the peptide includes two cationic domains.

In some embodiments, the peptide includes two cationic domains that are both arginine rich.

In some embodiments, the peptide includes one cationic domain that is arginine rich.

In some embodiments, the peptide includes two cationic domains that are both arginine and histidine rich.

In some embodiments, the peptide includes one cationic domain that is arginine rich and one cationic domain that is histidine rich.

In some embodiments, the cationic domains include amino acid units selected from the following: R, H, B, RR, HH, BB, RH, HR, RB, BR, HB, BH, RBR, RBB, BRR, BBR, BRB, RBH, RHB, HRB, BRH, HRR, RRH, HRH, HBB, BBH, RHR, BHB, HBH, or any combination thereof.

In some embodiments, a cationic domain may also include serine, proline, and/or hydroxyproline residues. In some embodiments, the cationic domains may further include amino acid units selected from the following: RP, PR, RPR, RRP, PRR, PRP, Hyp; R[Hyp]R, RR[Hyp], [Hyp]RR, [Hyp]R[Hyp], [Hyp][Hyp]R, R[Hyp][Hyp], SB, BS, or any combination thereof, or any combination with the above listed amino acid units.

In some embodiments, each cationic domain includes any one of the following sequences: RBRRBRR (SEQ ID NO: 1), RBRBR (SEQ ID NO: 2), RBRR (SEQ ID NO: 3), RBRRBR (SEQ ID NO: 4), RRBRBR (SEQ ID NO: 5), RBRRB (SEQ ID NO: 6), BRBR (SEQ ID NO: 7), RBHBH (SEQ ID NO: 8), HBHBR (SEQ ID NO: 9), RBRHBHR (SEQ ID NO: 10), RBRBBHR (SEQ ID NO: 11), RBRRBH (SEQ ID NO: 12), HBRRBR (SEQ ID NO: 13), HBHBH (SEQ ID NO: 14), BHBH (SEQ ID NO: 15), BRBSB (SEQ ID NO: 16), BRB[Hyp]B (SEQ ID NO: 17), R[Hyp]H[Hyp]HB (SEQ ID NO: 18), R[Hyp]RR[Hyp]R (SEQ ID NO: 19), or any combination thereof.

In some embodiments, each cationic domain consists of any one of the following sequences: RBRRBRR (SEQ ID NO: 1), RBRBR (SEQ ID NO: 2), RBRR (SEQ ID NO: 3), RBRRBR (SEQ ID NO: 4), RRBRBR (SEQ ID NO: 5), RBRRB (SEQ ID NO: 6), BRBR (SEQ ID NO: 7), RBHBH (SEQ ID NO: 8), HBHBR (SEQ ID NO: 9), RBRHBHR (SEQ ID NO: 10), RBRBBHR (SEQ ID NO: 11), RBRRBH (SEQ ID NO: 12), HBRRBR (SEQ ID NO: 13), HBHBH (SEQ ID NO: 14), BHBH (SEQ ID NO: 15), BRBSB (SEQ ID NO: 16), BRB[Hyp]B, R[Hyp]H[Hyp]HB, R[Hyp]RR[Hyp]R (SEQ ID NO: 19), or any combination thereof.

In some embodiments, each cationic domain consists of one of the following sequences: RBRRBRR (SEQ ID NO: 1), RBRBR (SEQ ID NO: 2), RBRRBR (SEQ ID NO: 4), BRBR (SEQ ID NO: 7), RBHBH (SEQ ID NO: 8), or HBHBR (SEQ ID NO: 9).

In some embodiments, each cationic domain in the peptide may be identical or different. In some embodiments, each cationic domain in the peptide is different.

### Hydrophobic Domain

The present invention relates to short cell-penetrating peptides having a particular structure in which there is at least one hydrophobic domain having a certain length.

References to ‘hydrophobic’ herein denote an amino acid or domain of amino acids having the ability to repel water or which do not mix with water.

In some embodiments, the peptide includes up to 3 hydrophobic domains, up to 2 hydrophobic domains. In some embodiments, the peptide includes 1 hydrophobic domain.

As defined above, the peptide includes one or more hydrophobic domains each having a length of at least 3 amino acid residues.

In some embodiments, each hydrophobic domain has a length of between 3-6 amino acids. In some embodiments, each hydrophobic domain has a length of 5 amino acids.

In some embodiments, each hydrophobic domain may include nonpolar, polar, and hydrophobic amino acid residues.

Hydrophobic amino acid residues may be selected from: alanine, valine, leucine, isoleucine, phenylalanine, tyrosine, methionine, and tryptophan.

Non-polar amino acid residues may be selected from: proline, glycine, cysteine, alanine, valine, leucine, isoleucine, tryptophan, phenylalanine, and methionine.

Polar amino acid residues may be selected from: serine, asparagine, hydroxyproline, histidine, arginine, threonine, tyrosine, and glutamine.

In some embodiments, the hydrophobic domains do not include hydrophilic amino acid residues.

In some embodiments, each hydrophobic domain includes a majority of hydrophobic amino acid residues. In some embodiments, each hydrophobic domain includes at least 70%, at least 75%, at least 80%, at least 85%, at least 90%, at least 95%, or 100% hydrophobic amino acids. In some embodiments, each hydrophobic domain consists of hydrophobic amino acid residues.

In some embodiments, each hydrophobic domain includes a hydrophobicity of at least 0.3, at least 0.4, at least 0.5, at least 0.6, at least 0.7, at least 0.8, at least 0.8, at least 1.0, at least 1.1, at least 1.2, or at least 1.3.

In some embodiments, each hydrophobic domain includes a hydrophobicity of at least 0.3, at least 0.35, at least 0.4, or at least 0.45.

In some embodiments, each hydrophobic domain includes a hydrophobicity of at least 1.2, at least 1.25, at least 1.3, or at least 1.35.

In some embodiments, each hydrophobic domain includes a hydrophobicity of between 0.4 and 1.4

In some embodiments, each hydrophobic domain includes of a hydrophobicity of between 0.45 and 0.48.

In some embodiments, each hydrophobic domain includes a hydrophobicity of between 1.27 and 1.39

In some embodiments, hydrophobicity is as measured by White and Wimley: W. C. Wimley and S. H. White, “Experimentally determined hydrophobicity scale for proteins at membrane interfaces” Nature Struct Biol 3:842 (1996).

In some embodiments, each hydrophobic domain includes at least 3 or at least 4 hydrophobic amino acid residues.

In some embodiments, each hydrophobic domain includes phenylalanine, leucine, Isoleucine, tyrosine, tryptophan, proline, and/or glutamine residues. In some embodiments, each hydrophobic domain consists of phenylalanine, leucine, isoleucine, tyrosine, tryptophan, proline, and/or glutamine residues.

In some embodiments, each hydrophobic domain consists of phenylalanine, leucine, isoleucine, tyrosine, and/or glutamine residues.

In some embodiments, each hydrophobic domain consists of tryptophan and/or proline residues.

In some embodiments, the peptide includes one hydrophobic domain. In some embodiments, the or each hydrophobic domain is located in the center of the peptide. In some embodiments, therefore, the hydrophobic domain may be known as a core hydrophobic domain. In some embodiments, the or each hydrophobic core domain is flanked on either side by an arm domain. In some embodiments, the arm domains may include one or more cationic domains and one or more further hydrophobic domains. In some embodiments, each arm domain includes a cationic domain.

In some embodiments, the peptide includes two arm domains flanking a hydrophobic core domain, where each arm domain includes a cationic domain.

In some embodiments, the peptide consists of two cationic arm domains flanking a hydrophobic core domain.

In some embodiments, the or each hydrophobic domain includes one of the following sequences: YQFLI (SEQ ID NO: 20), FQILY (SEQ ID NO: 21), ILFQY (SEQ ID NO: 22), FQIY (SEQ ID NO: 23), WWW, WWPWW (SEQ ID NO: 24), WPWW (SEQ ID NO: 25), WWPW (SEQ ID NO: 26), or any combination thereof.

In some embodiments, the or each hydrophobic domain consists of one of the following sequences: YQFLI (SEQ ID NO: 20), FQILY (SEQ ID NO: 21), ILFQY (SEQ ID NO: 22), FQIY (SEQ ID NO: 23), WWW, WWPWW (SEQ ID NO: 24), WPWW (SEQ ID NO: 25), WWPW (SEQ ID NO: 26), or any combination thereof.

In some embodiments, the or each hydrophobic domain consists of one of the following sequences FQILY (SEQ ID NO: 21), YQFLI (SEQ ID NO: 20), or ILFQY (SEQ ID NO: 22).

In some embodiments, the or each hydrophobic domain consists of FQILY (SEQ ID NO: 21).

In some embodiments, each hydrophobic domain in the peptide may have the same sequence or a different sequence.

The present invention relates to short cell-penetrating peptides for use in transporting therapeutic cargo molecules in the treatment of medical conditions.

The peptide has a sequence that is a contiguous single molecule, therefore the domains of the peptide are contiguous. In some embodiments, the peptide includes several domains in a linear arrangement between the N-terminus and the C-terminus. In some embodiments, the domains are selected from cationic domains and hydrophobic domains described above. In some embodiments, the peptide consists of cationic domains and hydrophobic domains where the domains are as defined above.

Each domain has common sequence characteristics as described in the relevant sections above, but the exact sequence of each domain is capable of variation and modification. Thus, a range of sequences is possible for each domain. The combination of each possible domain sequence yields a range of peptide structures, each of which form part of the present invention. Features of the peptide structures are described below.

In some embodiments, a hydrophobic domain separates any two cationic domains. In some embodiments, each hydrophobic domain is flanked by cationic domains on either side thereof.

In some embodiments, no cationic domain is contiguous with another cationic domain.

In some embodiments, the peptide includes one hydrophobic domain flanked by two cationic domains in the following arrangement:

[cationic domain]—[hydrophobic domain]—[cationic domain]

In some embodiments, the hydrophobic domain may be known as the core domain and each of the cationic domains may be known as an arm domain. In some embodiments, the hydrophobic arm domains flank the cationic core domain on either side thereof.

In some embodiments, the peptide consists of two cationic domains and one hydrophobic domain.

In some embodiments, the peptide consists of one hydrophobic core domain flanked by two cationic arm domains.

In some embodiments, the peptide consists of one hydrophobic core domain including a sequence selected from: YQFLI (SEQ ID NO: 20), FQILY (SEQ ID NO: 21), ILFQY (SEQ ID NO: 22), FQIY (SEQ ID NO: 23), WWW, WWPWW (SEQ ID NO: 24), WPWW (SEQ ID NO: 25), and WWPW (SEQ ID NO: 26), flanked by two cationic arm domains each including a sequence selected from: RBRRBRR (SEQ ID NO: 1), RBRBR (SEQ ID NO: 2), RBRR (SEQ ID NO: 3), RBRRBR (SEQ ID NO: 4), RRBRBR (SEQ ID NO: 5), RBRRB (SEQ ID NO: 6), BRBR (SEQ ID NO: 7), RBHBH (SEQ ID NO: 8), HBHBR (SEQ ID NO: 9), RBRHBHR (SEQ ID NO: 10), RBRBBHR (SEQ ID NO: 11), RBRRBH (SEQ ID NO: 12), HBRRBR (SEQ ID NO: 13), HBHBH (SEQ ID NO: 14), BHBH (SEQ ID NO: 15), BRBSB (SEQ ID NO: 16), BRB[Hyp]B (SEQ ID NO: 17), R[Hyp]H[Hyp]HB (SEQ ID NO: 18), and R[Hyp]RR[Hyp]R (SEQ ID NO: 19).

In some embodiments, the peptide consists of one hydrophobic core domain including a sequence selected from: FQILY (SEQ ID NO: 21), YQFLI (SEQ ID NO: 20), and ILFQY (SEQ ID NO: 22), flanked by two cationic arm domains including a sequence selected from: RBRRBRR (SEQ ID NO: 1), RBRBR (SEQ ID NO: 2), RBRRBR (SEQ ID NO: 4), BRBR (SEQ ID NO: 7), RBHBH (SEQ ID NO: 8), and HBHBR (SEQ ID NO: 9). In some embodiments, the peptide consists of one hydrophobic core domain including the sequence: FQILY (SEQ ID NO: 21), flanked by two cationic arm domains including a sequence selected from: RBRRBRR (SEQ ID NO: 1), RBRBR (SEQ ID NO: 2), RBRRBR (SEQ ID NO: 4), BRBR (SEQ ID NO: 7), and RBHBH (SEQ ID NO: 8).

In any such embodiment, further groups may be present such as a linker, terminal modification, and/or oligonucleotide.

In some embodiments, the peptide is N-terminally modified.

In some embodiments, the peptide is N-acetylated, N-methylated, N-trifluoroacetylated, N-trifluoromethylsulfonylated, or N-methylsulfonylated. In some embodiments, the peptide is N-acetylated.

Optionally, the N-terminus of the peptide may be unmodified.

In some embodiments, the peptide is N-acetylated.

In some embodiments, the peptide is C-terminal modified.

In some embodiments, the peptide includes a C-terminal modification selected from: carboxy-, thioacid-, aminooxy-, hydrazino-, thioester-, azide, strained alkyne, strained alkene, aldehyde-, thiol, or haloacetyl-group.

Advantageously, the C-terminal modification provides a means for linkage of the peptide to the oligonucleotide.

Accordingly, the C-terminal modification may include the linker and vice versa. In some embodiments, the C-terminal modification may consist of the linker or vice versa. Suitable linkers are described herein elsewhere.

In some embodiments, the peptide includes a C-terminal carboxyl group.

In some embodiments, the C-terminal carboxyl group is provided by a glycine or beta-alanine residue.

In some embodiments, the C terminal carboxyl group is provided by a beta-alanine residue. In some embodiments, the C terminal beta-alanine residue is a linker. In some embodiments, the C terminal glutamic acid (with a free —COON replaced with —CONH₂) is a linker. In some embodiments, the conjugate is of the following structure:

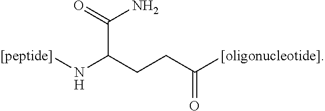

In some embodiments, therefore each cationic domain may further include an N or C terminal modification. In some embodiments, the cationic domain at the C terminus includes a C-terminal modification. In some embodiments, the cationic domain at the N terminus includes a N-terminal modification. In some embodiments, the cationic domain at the C terminus includes a linker group. In some embodiments, the cationic domain at the C terminus includes a C-terminal beta-alanine. In some embodiments, the cationic domain at the N terminus is N-acetylated.

The peptide of the present invention is defined as having a total length of 40 amino acid residues or less. The peptide may therefore be regarded as an oligopeptide.

In some embodiments, the peptide has a total length of 3-30 amino acid residues, e.g., of 5-25 amino acid residues, of 10-25 amino acid residues, of 13-23 amino acid residues, or of 15-20 amino acid residues.

In some embodiments, the peptide has a total length of at least 12, at least 13, at least 14, at least 15, at least 16, or at least 17 amino acid residues.

In some embodiments, the peptide is capable of penetrating cells. The peptide may therefore be regarded as a cell-penetrating peptide.

In some embodiments, the peptide is for attachment to an oligonucleotide. In some embodiments, the peptide is for transporting an oligonucleotide into a target cell. In some embodiments, the peptide is for delivering an oligonucleotide into a target cell. The peptide may therefore be regarded as a carrier peptide.

In some embodiments, the peptide is capable of penetrating into cells and tissues, e.g., into the nucleus of cells. In some embodiments, into muscle tissues.

In some embodiments, the peptide may be selected from any one of the following sequences:

(SEQ ID NO: 27)
RBRRBRRFQILYRBRBR
(SEQ ID NO: 28)
RBRRBRRFQILYRBRR
(SEQ ID NO: 29)
RBRRBRFQILYRRBRBR
(SEQ ID NO: 30)
RBRBRFQILYRBRRBRR
(SEQ ID NO: 31)
RBRRBRRYQFLIRBRBR
(SEQ ID NO: 32)
RBRRBRRILFQYRBRBR
(SEQ ID NO: 33)
RBRRBRFQILYRBRBR
(SEQ ID NO: 34)
RBRRBFQILYRBRRBR
(SEQ ID NO: 35)
RBRRBRFQILYBRBR
(SEQ ID NO: 36)
RBRRBFQILYRBRBR
(SEQ ID NO: 37)
RBRRBRRFQILYRBHBH
(SEQ ID NO: 38)
RBRRBRRFQILYHBHBR
(SEQ ID NO: 39)
RBRRBRRFQILYHBRBH
(SEQ ID NO: 40)
RBRRBRRYQFLIRBHBH
(SEQ ID NO: 41)
RBRRBRRILFQYRBHBH
(SEQ ID NO: 42)
RBRHBHRFQILYRBRBR
(SEQ ID NO: 43)
RBRBBHRFQILYRBHBH
(SEQ ID NO: 44)
RBRRBRFQILYRBHBH
(SEQ ID NO: 45)
RBRRBRFQILYHBHBH
(SEQ ID NO: 46)
RBRRBHFQILYRBHBH
(SEQ ID NO: 47)
HBRRBRFQILYRBHBH
(SEQ ID NO: 48)
RBRRBFQILYRBHBH
(SEQ ID NO: 49)
RBRRBRFQILYBHBH
(SEQ ID NO: 50)
RBRRBRYQFLIHBHBH
(SEQ ID NO: 51)
RBRRBRILFQYHBHBH
(SEQ ID NO: 52)
RBRRBRRFQILYHBHBH

In some embodiments, the peptide may be selected from any one of the following additional sequences:

(SEQ ID NO: 53)
RBRRBRFQILYBRBS
(SEQ ID NO: 54)
RBRRBRFQILYBRB[Hyp]
(SEQ ID NO: 55)
RBRRBRFQILYBR[Hyp]R
(SEQ ID NO: 56)
RRBRRBRFQILYBRBR
(SEQ ID NO: 57)
BRRBRRFQILYBRBR
(SEQ ID NO: 58)
RBRRBRWWWBRBR
(SEQ ID NO: 59)
RBRRBRWWPWWBRBR
(SEQ ID NQ:60)
RBRRBRWPWWBRBR
(SEQ ID NO: 61)
RBRRBRWWPWBRBR
(SEQ ID NO: 62)
RBRRBRRWWWRBRBR
(SEQ ID NO: 63)
RBRRBRRWWPWWRBRBR
(SEQ ID NO: 64)
RBRRBRRWPWWRBRBR
(SEQ ID NO: 65)
RBRRBRRWWPWRBRBR
(SEQ ID NO: 66)
RBRRBRRFQILYBRBR
(SEQ ID NO: 67)
RBRRBRRFQILYRBR
(SEQ ID NO: 68)
BRBRBWWPWWRBRRBR
(SEQ ID NO: 69)
RBRRBRRFQILYBHBH
(SEQ ID NO: 70)
RBRRBRRFQIYRBHBH
(SEQ ID NO: 71)
RBRRBRFQILYBRBH
(SEQ ID NO: 72)
RBRRBRFQILYR[Hyp]H[Hyp]H
(SEQ ID NO: 73)
R[Hyp]RR[Hyp]RFQILYRBHBH
(SEQ ID NO: 74)
R[Hyp]RR[Hyp]RFQILYR[Hyp]H[Hyp]H
(SEQ ID NO: 75)
RBRRBRWWWRBHBH
(SEQ ID NO: 76)
RBRRBRWWPRBHBH
(SEQ ID NO: 77)
RBRRBRPWWRBHBH
(SEQ ID NO: 78)
RBRRBRWWPWWRBHBH
(SEQ ID NO: 79)
RBRRBRWWPWRBHBH
(SEQ ID NO: 80)
RBRRBRWPWWRBHBH
(SEQ ID NO: 81)
RBRRBRRWWWRBHBH
(SEQ ID NO: 82)
RBRRBRRWWPWWRBHBH
(SEQ ID NO: 83)
RBRRBRRWPWWRBHBH
(SEQ ID NO: 84)
RBRRBRRWWPWRBHBH
(SEQ ID NO: 85)
RRBRRBRFQILYRBHBH
(SEQ ID NO: 86)
BRRBRRFQILYRBHBH
(SEQ ID NO: 87)
RRBRRBRFQILYBHBH
(SEQ ID NO: 88)
BRRBRRFQILYBHBH
(SEQ ID NO: 89)
RBRRBHRFQILYRBHBH
(SEQ ID NO: 101)
RBRRBRFQILY[Hyp]R[Hyp]R
(SEQ ID NO: 102)
R[Hyp]RR[Hyp]RFQILYBRBR
(SEQ ID NO: 103)
R[Hyp]RR[Hyp]RFQILY[Hyp]R[Hyp]R
(SEQ ID NO: 104)
RBRRBRWWWBRBR
(SEQ ID NO: 105)
RBRRBRWWPWWBRBR

In some embodiments, the peptide may be selected from one of the following sequences:

(SEQ ID NO: 27)
RBRRBRRFQILYRBRBR
(SEQ ID NO: 31)
RBRRBRRYQFLIRBRBR
(SEQ ID NO: 32)
RBRRBRRILFQYRBRBR
(SEQ ID NO: 35)
RBRRBRFQILYBRBR
(SEQ ID NO: 37)
RBRRBRRFQILYRBHBH
(SEQ ID NO: 38)
RBRRBRRFQILYHBHBR
(SEQ ID NO: 44)
RBRRBRFQILYRBHBH

In some embodiments, the peptide consists of the following sequence: RBRRBRFQILYBRBR (SEQ ID NO: 35).

In some embodiments, the peptide consists of the following sequence: RBRRBRRFQILYRBHBH (SEQ ID NO: 37).

In some embodiments, the peptide consists of the following sequence: RBRRBRFQILYRBHBH (SEQ ID NO: 44).

### Conjugate

In some embodiments, the conjugate includes a peptide selected from one of the following sequences: RBRRBRFQILYBRBR (SEQ ID NO: 35), RBRRBRRFQILYRBHBH (SEQ ID NO: 37) and RBRRBRFQILYRBHBH (SEQ ID NO: 44).

In some embodiments, in any case, the peptide may further include N-terminal modifications as described above.

Preferably, the antisense oligonucleotide is a phosphorodiamidate morpholino oligonucleotide (PMO). Alternatively the oligonucleotide may be a modified PMO or any other charge-neutral oligonucleotide such as a peptide nucleic acid (PNA), a chemically modified PNA such as a gammaPNA (Bahal, Nat. Comm. 2016), oligonucleotide phosphoramidate (where the non-bridging oxygen of the phosphate is substituted by an amine or alkylamine such as those described in WO2016028187A1), or any other partially or fully charge-neutralized oligonucleotide.

Suitable linkers include, for example, a C-terminal cysteine residue that permits formation of a disulphide, thioether or thiol-maleimide linkage, a C-terminal aldehyde to form an oxime, a click reaction or formation of a morpholino linkage with a basic amino acid on the peptide or a carboxylic acid moiety on the peptide covalently conjugated to an amino group to form a carboxamide linkage.

In some embodiments, the linker is between 1-5 amino acids in length. In some embodiments, the linker may include any linker that is known in the art. In some embodiments, the linker is selected from any of the following sequences: G, BC, XC, C, GGC, BBC, BXC, XBC, X, XX, B, BB, BX and XB. In some embodiments, where X is 6-aminohexanoic acid. In some embodiments the linker is a Glu linker.

In some embodiments, the linker may be a polymer, such as for example PEG.

In some embodiments, the linker is beta-alanine.

In some embodiments, the peptide is conjugated to the oligonucleotide through a carboxamide linkage.

The linker of the conjugate may form part of the oligonucleotide to which the peptide is attached.

Alternatively, the attachment of the oligonucleotide may be directly linked to the C-terminus of the peptide. In some embodiments, in such embodiments, no linker is required.

Alternatively, the peptide may be chemically conjugated to the oligonucleotide. Chemical linkage may be via a disulphide, alkenyl, alkynyl, aryl, ether, thioether, triazole, amide, carboxamide, urea, thiourea, semicarbazide, carbazide, hydrazine, oxime, phosphate, phosphoramidate, thiophosphate, boranophosphate, iminophosphates, or thiol-maleimide linkage, for example.

Optionally, cysteine may be added at the N-terminus of a peptide to allow for disulphide bond formation to the peptide, or the N-terminus may undergo bromoacetylation for thioether conjugation to the peptide.

In some embodiments, the conjugate is capable of penetrating into cells and tissues, e.g., into the nucleus of cells, e.g., into muscle tissues.

In some embodiments, the oligonucleotide component of the conjugate is a PMO.

In some embodiments, the oligonucleotide component of the conjugate is an oligonucleotide as described herein, such as in the “oligonucleotide” section above or elsewhere herein.

### Linkers

In addition to the above, conjugates described herein may include a linker covalently linking a peptide described herein to an oligonucleotide described herein. Linkers useful in the present invention can be found in WO 2020/115494, the disclosure of which is incorporated herein by reference.

The linker may be of formula (I):

T₁—(CR¹R²)ₙ—T₂. (I)

where

T₁ is a divalent group for attachment to the peptide and is selected from the group consisting of —NH- and carbonyl;

T₂ is a divalent group for attachment to an oligonucleotide and is selected from the group consisting of —NH- and carbonyl;

n is 1, 2 or 3;

each R¹ is independently -Y¹-X¹-Z¹,

where
Y¹ is absent or —(CR^{A1}R^{A2})ₘ-, where m is 1, 2, 3 or 4, and R^{A1} and R^{A2} are each independently hydrogen, OH, or (1-2C)alkyl;
X¹ is absent, —O—, —C(O)—, —C(O)O—, —OC(O)—, —CH(OR^{A3})—, —N(R^{A3})—, —N(R^{A3})—C(O)—, —N(R^{A3})—C(O)O—, —C(O)—N(R^{A3})—, —N(R^{A3})C(O)N(R^{A3})—, —N(R^{A3})C(NR^{A3})N(R^{A3})—, —SO—, —S—, —SO₂—, —S(O)₂N(R^{A3})-, or —N(R^{A3})SO₂-, where each R^{A3} is independently selected from hydrogen and methyl; and
Z¹ is a further oligonucleotide or is hydrogen, (1-6C)alkyl, (2-6C)alkenyl, (2-6C)alkynyl, aryl, (3-6C)cycloalkyl, (3-6C)cycloalkenyl, or heteroaryl, where each (1 -6C)alkyl, (2-6C)alkenyl, (2-6C)alkynyl, aryl, (3-6C)cycloalkyl, (3-6C)cycloalkenyl, and heteroaryl is optionally substituted with one or more (e.g., 1, 2, 3, 4, or 5) substituent groups selected from the group consisting of (1-4C) alkyl, oxo, halo, cyano, nitro, hydroxy, carboxy, NR^{A4}R^{A5}, and (1-4C)alkoxy, where R^{A4} and R^{A5} are each independently selected from the group consisting of hydrogen and (1-4C)alkyl; and

each R² is independently -Y²-X²-Z², where

Y² is absent or a group of the formula —[CR^{B1}R^{B2}]ₘ- in which m is an integer selected from 1, 2, 3 or 4, and R^{B1} and R^{B2} are each independently selected from hydrogen, OH or (1-2C)alkyl;
X² is absent, —O—, —C(O)—, —C(O)O—, —OC(O)—, —CH(OR^{B3})—, —N(R^{B3})—, —N(R^{B3})—C(O)—, —N(R^{B3})—C(O)O—, —C(O)—N(R^{B3})—, —N(R^{B3})C(O)N(R^{B3})—, —N(R^{B3})C(NR^{B3})N(R^{B3})—, —SO—, —S— —SO₂—, —S(O)₂N(R^{B3})-, or —N(R^{B3})SO₂-,
where each R^{B3} is independently selected from hydrogen or methyl; and
Z² is selected from hydrogen, (1 -6C)alkyl, (2-6C)alkenyl, (2-6C)alkynyl, aryl, (3- 6C)cycloalkyl, (3-6C)cycloalkenyl or heteroaryl, where each (1 -6C)alkyl, (2- 6C)alkenyl, (2-6C)alkynyl, aryl, (3-6C)cycloalkyl, (3-6C)cycloalkenyl or heteroaryl is optionally substituted by one or more (e.g., 1, 2, 3, 4, or 5) substituent groups selected from the group consisting of (1-4C) alkyl, oxo, halo, cyano, nitro, hydroxy, carboxy, NR^{B4}R^{B5}, and (1-4C)alkoxy, where R^{B4} and R^{B5} are each independently hydrogen or (1-2C)alkyl; with the proviso that; when n=1 and T₁ and T² are different to one another, then R¹ and R² are not both H; when n=1, T¹ and T² are different to one another and one of R¹ and R² is H then the other of R¹ and R² is not methyl; or when n=2 and each occurrence of R¹ and R² is H, then T¹ and T² are both —C(O)- or are both —NH-.

In some embodiments, the linker is of the following structure:

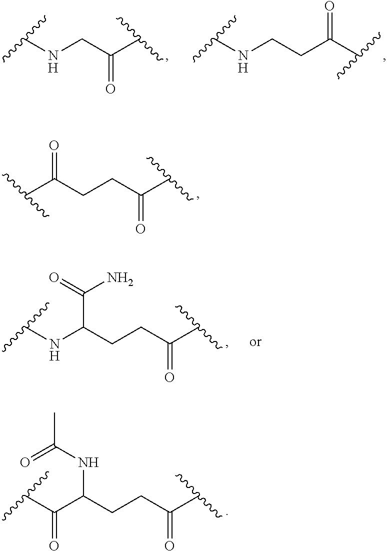 [SMILES: *NCC(*)=O ; *NCCC(*)=O ; *C(=O)CCC(*)=O ; *NC(CCC(*)=O)C(N)=O ; *C(=O)CCC(NC(C)=O)C(*)=O]

### Pharmaceutical Compositions

The conjugate of the invention, or a pharmaceutically acceptable salt thereof, may formulated into a pharmaceutical composition.

In some embodiments, the pharmaceutical composition includes a conjugate of the invention or a pharmaceutically acceptable salt thereof.

In some embodiments, the pharmaceutical composition may further include a pharmaceutically acceptable diluent, adjuvant or carrier.

Suitable pharmaceutically acceptable diluents, adjuvants and carriers are well known in the art.

It should be understood that the pharmaceutical compositions of the present disclosure can further include additional known therapeutic agents, drugs, modifications of compounds into prodrugs, and the like for alleviating, mediating, preventing, and treating the diseases, disorders, and conditions described herein under medical use.

In some embodiments, the pharmaceutical composition is for use as a medicament, e.g., for use as a medicament in the same manner as described herein for the conjugate. All features described herein in relation to medical treatment using the conjugate apply to the pharmaceutical composition.

Accordingly, in a further aspect of the invention there is provided a pharmaceutical composition according to the fourth aspect for use as a medicament. In a further aspect, there is provided a method of treating a subject for a disease condition including administering an effective amount of a pharmaceutical composition disclosed herein.

### Medical Use

The conjugate including the peptide of the invention may be used as a medicament for the treatment of a disease using the administration regimen described herein.

The medicament may be in the form of a pharmaceutical composition as defined above.

A method of treatment of a patient or subject in need of treatment for a disease condition is also provided, the method including the step of administering a therapeutically effective amount of the conjugate to the patient or subject. In some embodiments, the medical treatment requires delivery of the oligonucleotide into a cell, e.g., into the nucleus of the cell.

Diseases to be treated may include any disease where improved penetration of the cell and/or nuclear membrane by an oligonucleotide may lead to an improved therapeutic effect.

In some embodiments, the conjugate is for use in the treatment of diseases of the neuromuscular system.

In some embodiments, the conjugate is for use in the treatment of diseases caused by splicing deficiencies. In such embodiments, the oligonucleotide may include an oligonucleotide capable of preventing or correcting the splicing defect and/or increasing the production of correctly spliced mRNA molecules.

In some embodiments, there is provided a conjugate according to the second aspect for use in the treatment of DM1.

In some embodiments, in such an embodiment, the oligonucleotide of the conjugate is operable to reduce mis-splicing events and/or myotonia caused by the trinucleotide repeat expansion of the DMPK gene. In some embodiments, the oligonucleotide of the conjugate is operable to normalize splicing events and/or myotonia.

In some embodiments, in such an embodiment, the oligonucleotide of the conjugate is operable to reverse splicing defects and myotonia resulting from the of pathological DMPK gene repeat expansions.

In some embodiments, the conjugate reduces DM1-related mis-splicing defects by 10%, 15%, 20%, 25%, 30%, 35%,40%, 45%, 50%, 55%, 60%, 65%, or 70%. In some embodiments, the conjugate reduces DM1-related mis-splicing defects by up to 50%.

In some embodiments, the conjugate reverses splicing defects and myotonia resulting from the of pathological DMPK gene repeat expansions by up to 50%.

In some embodiments, the oligonucleotide of the conjugate is operable to do so by causing reversal of one or more of the multi-splicing defects and myotonia resulting from the of pathological DMPK gene repeat expansions.

In some embodiments, the oligonucleotide of the conjugate causes 10%, 15%, 20%, 25%, 30%, 35%, 40%, 45%, 50%, 55%, 60%, 65%, 70%, 75%, 80%, or 85% skipping of one or more exons of mis-spliced transcripts. In some embodiments, the oligonucleotide of the conjugate causes up to 50% reversal of one or more of the multi-splicing defects and myotonia resulting from the of pathological DMPK gene repeat expansions.

In some embodiments, the patient or subject to be treated may be any animal or human. In some embodiments, the patient or subject may be a non-human mammal. In some embodiments, the patient or subject may be male or female.

In some embodiments, the patient or subject to be treated may be any age. In some embodiments, the patient or subject to be treated is aged between 0-70 years, 0-60 years, 0-50 years, 0-40 years, in some embodiments, 0-30, in some embodiments, 0-25, in some embodiments, or 0-20 years of age.

In some embodiments, the conjugate is for administration to a subject systemically for example by intramedullary, intrathecal, intraventricular, intravitreal, enteral, parenteral, intravenous, intra-arterial, intramuscular, intratumoral, subcutaneous oral or nasal routes.

In some embodiments, the conjugate is for administration to a subject intravenously.

In some embodiments, the conjugate is for administration to a subject intravenously by injection.

In some embodiments, the conjugate is for administration to a subject intravenously by infusion.

Advantageously, the dosage of the conjugates of the present invention may be lower, e.g., an order or magnitude lower, than the dosage required to see any effect from the oligonucleotide alone.

In some embodiments, after administration of the conjugates of the present invention, one or more markers of toxicity are significantly reduced compared to prior conjugates using currently available peptide carriers

Suitable markers of toxicity may be markers of nephrotoxicity.

Suitable markers of toxicity include KIM-1, NGAL, BUN, creatinine, alkaline phosphatase, alanine transferase, and aspartate aminotransferase.

In some embodiments, the level of at least one of KIM-1, NGAL, and BUN is reduced after administration of the conjugates of the present invention when compared to prior conjugates using currently available peptide carriers.

In some embodiments, the levels of each of KIM-1, NGAL, and BUN are reduced after administration of the conjugates of the present invention when compared to prior conjugates using currently available peptide carriers.

In some embodiments, the levels of the or each marker is significantly reduced when compared to prior conjugates using currently available peptide carriers.

In some embodiments, the levels of the or each marker/s is reduced by up to 5%, 10%, 15%, 20%, 25%, 30%, 35%, 40%, 45%, or 50% after administration of the conjugates of the present invention when compared to prior conjugates using currently available peptide carriers.

In some embodiments, each dose within the plurality of doses being administered includes 5-60 mg/kg of the conjugate.

In some embodiments, each dose within the plurality of doses being administered includes 40 mg/kg to 60 mg/kg, 30 mg/kg to 50 mg/kg, 30 mg/kg to 40 mg/kg, 40 mg/kg to 50 mg/kg, 50 mg/kg to 60 mg/kg, 35 mg/kg to 45 mg/kg, 45 mg/kg to 55 mg/kg, 35 mg/kg to 55 mg/kg, 30 mg/kg to 45 mg/kg, 35 mg/kg to 50 mg/kg, 40 mg/kg to 55 mg/kg, 45 mg/kg to 60 mg/kg, 1 mg/kg to 30 mg/kg, 1 mg/kg to 20 mg/kg, 5 mg/kg to 25 mg/kg, 10 mg/kg to 30 mg/kg, 1 mg/kg to 15 mg/kg, 5 mg/kg to 20 mg/kg, 10 mg/kg to 25 mg/kg, 15 mg/kg to 30 mg/kg, 1 mg/kg to 10 mg/kg, 5 mg/kg to 15 mg/kg, 10 mg/kg to 20 mg/kg, 15 mg/kg to 25 mg/kg, 20 mg/kg to 30 mg/kg, 1 mg/kg to 25 mg/kg, 4 mg/kg to 20 mg/kg, 6 mg/kg to 15 mg/kg, or 8 mg/kg to 10 mg/kg of the conjugate.

In some embodiments, each dose within the plurality of doses being administered includes 1 mg/kg, 4 mg/kg, 5 mg/kg, 6 mg/kg, 8 mg/kg, 10 mg/kg, 15 mg/kg, 20 mg/kg, 25 mg/kg, 30 mg/kg, 35 mg/kg, 40 mg/kg, 45 mg/kg, 50 mg/kg, or 60 mg/kg of the conjugate.

The regimen used can be, e.g., as described elsewhere herein (see, e.g., the beginning of the Detailed Description). Accordingly, in some embodiments, the therapeutic regimen comprises a plurality of doses of a conjugate as described herein spaced at a time interval of at least 1 month, e.g., about 1-6, 2-6, 3-6, 4-6, or 5-6 months, or the interval is about 1, 2, 3, 4, 5, or 6 months. In some embodiments, the methods further comprise a treatment initiation regimen comprising administering a conjugate described herein three or four times at an initiation interval of about 2 weeks. It is to be understood that an interval or time period described as “about” an indicated month number can vary by, e.g., 1, 2, 3, 4, 5, 6, or 7 days from the precise indication. Similarly, it is to be understood that an interval or time period described as “about” an indicated week number can vary by, e.g., 1, 2, or 3 days.

### Peptide Preparation

Peptides of the invention may be produced by any standard protein synthesis method, for example chemical synthesis, semi-chemical synthesis or through the use of expression systems. Accordingly, the present invention also relates to the nucleotide sequences comprising or consisting of the DNA coding for the peptides, expression systems, e.g., vectors comprising said sequences accompanied by the necessary sequences for expression and control of expression, and host cells and host organisms transformed by said expression systems.

Accordingly, a nucleic acid encoding a peptide according to the present invention is also provided.

In some embodiments, the nucleic acids may be provided in isolated or purified form.

An expression vector comprising a nucleic acid encoding a peptide according to the present invention is also provided.

In some embodiments, the vector is a plasmid.

In some embodiments, the vector comprises a regulatory sequence, e.g., promoter, operably linked to a nucleic acid encoding a peptide according to the present invention. In some embodiments, the expression vector is capable of expressing the peptide when transfected into a suitable cell, e.g., mammalian, bacterial, or fungal cell.

A host cell comprising the expression vector of the invention is also provided.

Expression vectors may be selected depending on the host cell into which the nucleic acids of the invention may be inserted. Such transformation of the host cell involves conventional techniques such as those taught in Sambrook et al., Molecular Cloning: A Laboratory Manual, Cold Spring Harbor Laboratory Press, NY, USA, 2001. Selection of suitable vectors is within the skills of the person knowledgeable in the field. Suitable vectors include plasmids, bacteriophages, cosmids, and viruses.

The peptides produced may be isolated and purified from the host cell by any suitable method e.g. precipitation or chromatographic separation e.g. affinity chromatography.

Suitable vectors, hosts, and recombinant techniques are well known in the art.

The following examples are meant to illustrate the invention. They are not meant to limit the invention in any way.

### EXAMPLES

The conjugate studied in the Examples described herein is of the following structure

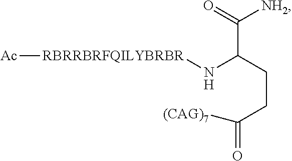

where the 5′ group is

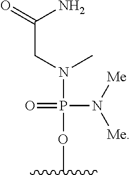 [SMILES: *OP(=O)(N(C)C)N(C)CC(N)=O]

### PPMO Conjugate

The internucleoside linkages in the conjugate are —P(═O)(NMe₂)—O-. This conjugate can be used in any of the methods described herein, e.g., as set forth in the claims.

### Example 1

### Removal of Physiological and Molecular Phenotype in Skeletal Muscle Following Single Intravenous Delivery of PPMO in HSALR Mice, the Myotonic Dystrophy Type One Mouse Model

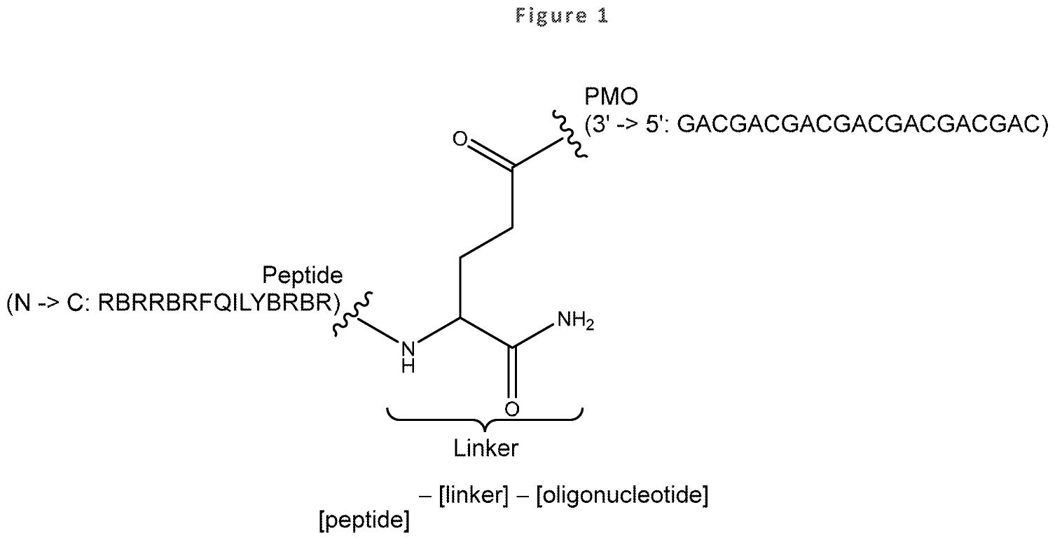

We tested the in vivo administration of PPMO conjugate (see FIG. 1).

The antisense oligonucleotide was specifically directed at treating DM1 by targeting the toxic trinucleotide repeat expansion found in the DMPK gene. HSA^{LR} mice were treated at 8-11 weeks of age with a single intravenous tail vein administration across a dose range of 10 mg/kg and 30 mg/kg of PPMO conjugate. Saline was used for control purposes in both HSA^{LR} mice and control wild type (WT) FVB mice. Under anesthetic conditions, myotonia was measured in the skeletal muscle two weeks post administration, and subsequently serum and tissues were harvested.

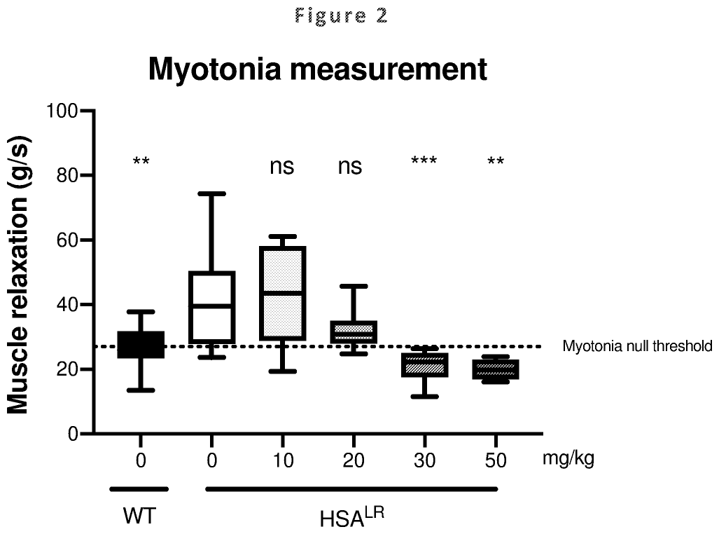

For comparison of PPMO conjugate impact on muscle physiology, myotonia measurements were assessed in saline-treated WT and HSA^{LR} mice and PPMO conjugate treated HSA^{LR} mice. A single administration of PPMO conjugate to HSA^{LR} mice at 10, 20, 30, and 50 mg/kg induced minor improvements on myotonia levels, while a single administration of PPMO conjugate to the HSALR mice at 30 and 50 mg/kg successfully normalized myotonia to WT levels in a statistically significant manner (see FIG. 2). It is clear from this data that a single administration of PPMO conjugate at 30 mg/kg or greater has the ability to correct the myotonic phenotype.

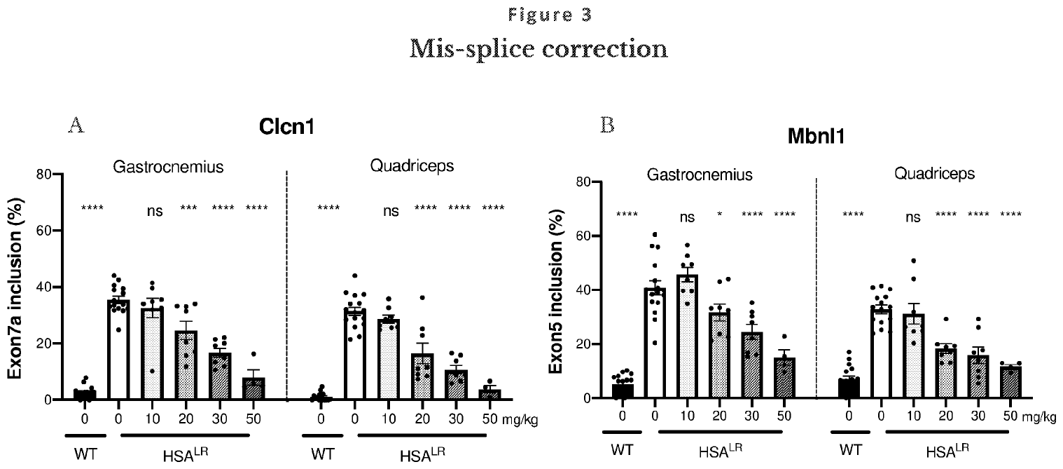

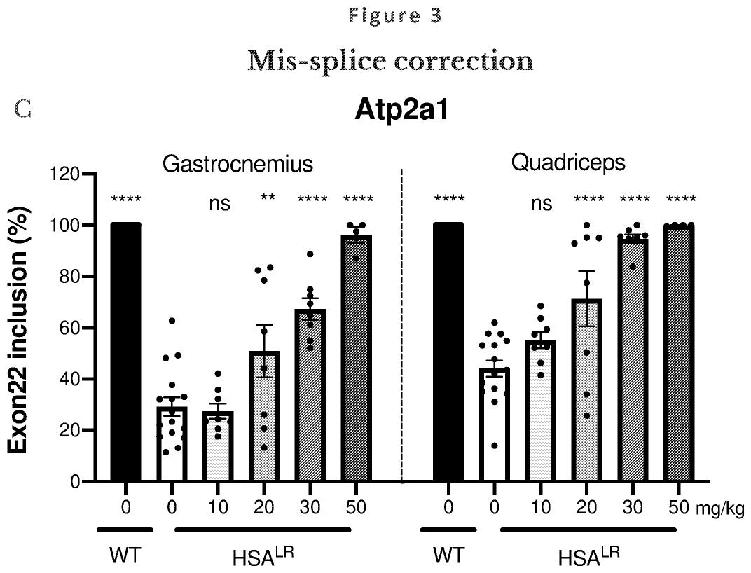

For the molecular level comparison of the impact of PPMO conjugates on splice correction, analysis was performed on extracted RNA by RT-PCR for key HSA^{LR} mis-splicing events (Clcn1, Mbnl1, and Atp2a1) in both the gastrocnemius and the quadriceps muscle. Administration of a single dose of PPMO conjugate to HSA^{LR} mice at 10 mg/kg induced slight improvements on mis-splice correction of Clon1, MbnI1, and Atp2a1 transcripts, while a single administration of PPMO conjugate to the HSALR mice at 20, 30, and 50 mg/kg had significant improvements on mis-splice correction of the same transcripts, returning levels to more than 75% correction when compared to WT levels in gastrocnemius and quadriceps skeletal muscle (FIG. 3). It is clear from this data that a single administration of PPMO conjugate at 20 mg/kg or more has the ability to significantly improve mis-splice correction of key transcripts in the HSALR mouse model of myotonic dystrophy type 1.

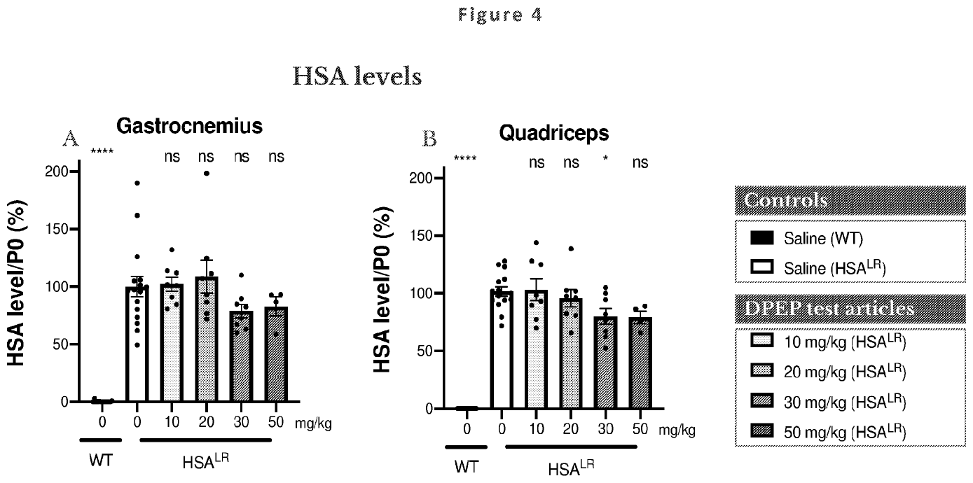

Further molecular analysis was performed by qPCR to assess the levels of CUGexp HSA transcripts in the gastrocnemius (FIG. 4a) and quadriceps (FIG. 4b) muscles of HSALR mice after administration of a single dose of PPMO conjugate at 10, 20, 30, or 50 mg/kg. PPMO conjugate treatment in HSA^{LR} mice induced no significant change in CUGexp HSA transcript levels normalized to PO in gastrocnemius (FIG. 4a) and quadriceps skeletal muscle (FIG. 4b) at all doses tested. Administration of PPMO conjugate is thus not seen to change levels of HSA transcript expression in the HSA^{LR} DM1 mouse model.

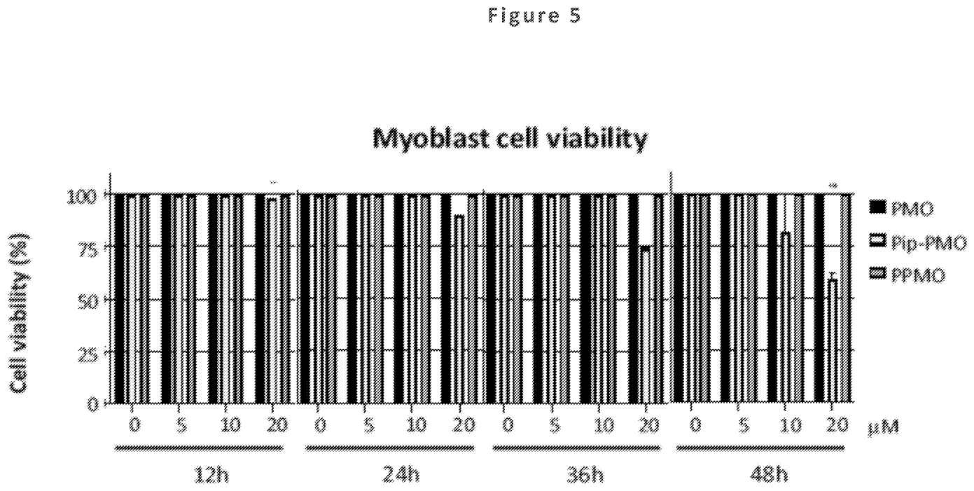

Viability of human myoblasts in vitro was measured at 12, 24, 36, and 48 hours after exposure to PPMO conjugate, Pip-conjugate PMO (Pip-PMO), or unconjugated PMO (FIG. 5). Treatment of myoblasts with PPMO conjugate at concentrations up to and including 20 μM caused no measurable decline in myoblast viability. PPMO can be administered as concentrations increased several-fold above therapeutic levels without causing cell death in myoblasts.

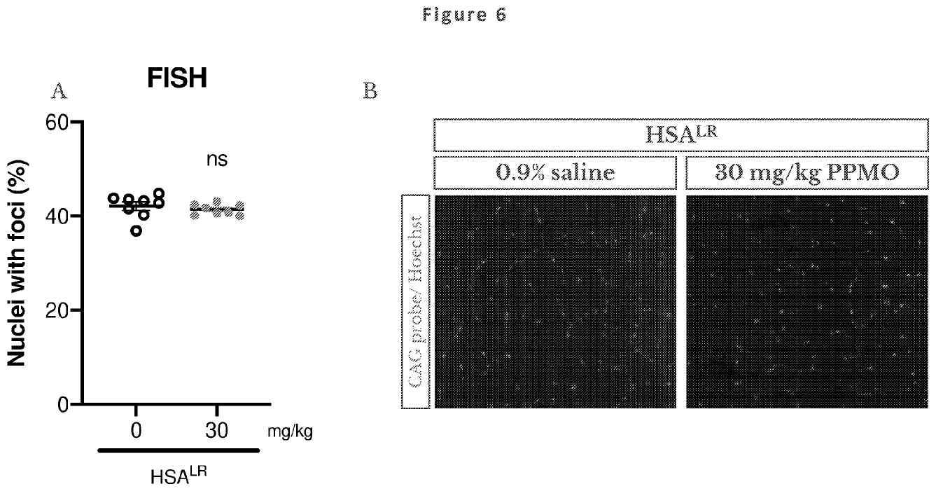

In further studies, we found that PMO^{DM1} targets CUG repeat and works through steric blocking. We further found that PPMO conjugate has no impact on nuclear foci numbers in gastrocnemius muscle. These studies were carried out by FISH analysis using a CAG probe as shown in FIG. 6.

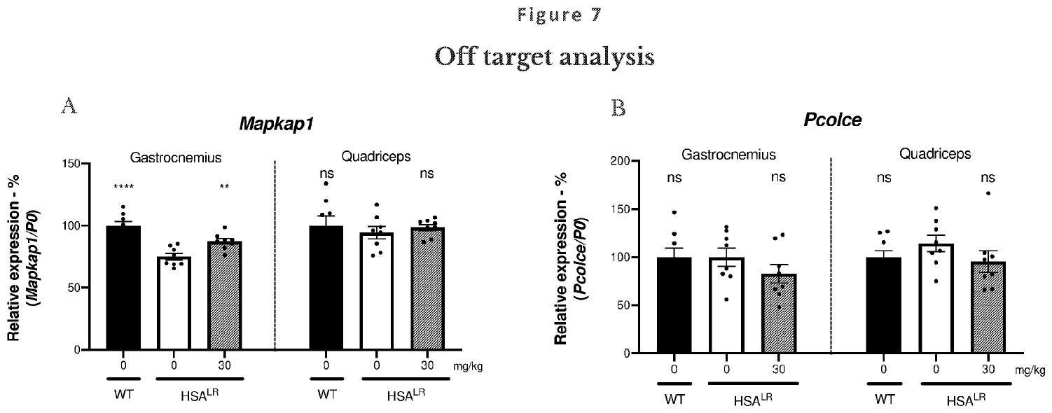

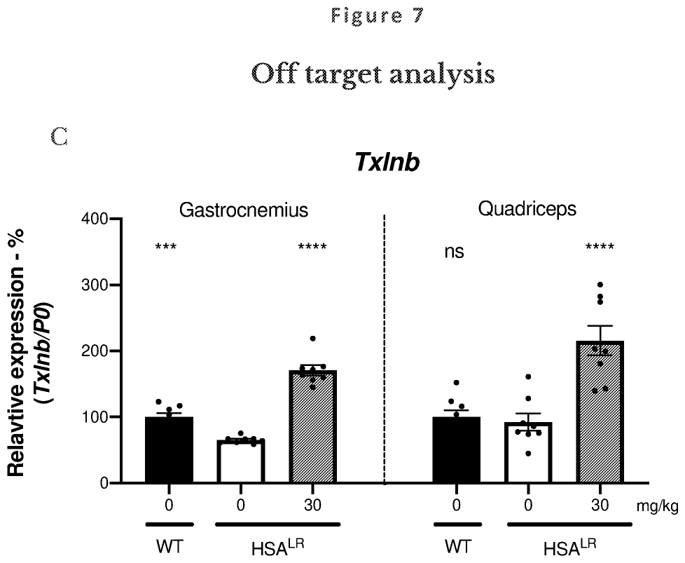

We also carried out off-target analysis in order to assess the impact of a repeat sequence PMO on naturally occurring CUG repeats. As shown in FIG. 7, PPMO conjugate has no significant effects on Mapkap1 or Pcolce, whereas the level of Txlnb transcript is moderately elevated as compared to baseline.

Further experiments to assess the safety of PPMO conjugate were carried out. Levels of urea, creatinine, creatine kinase, albumin, alkaline phosphatase (ALP), alanine transferase (ALT), and aspartate aminotransferase (AST) were measured in serum of HSA^{LR} mice after administration of PPMO conjugate at 10, 20, 30, and 50 mg/kg. Measured levels urea, creatinine, ALP, ALT, AST, albumin, and creatine kinase levels were similar at all doses of PPMO conjugate and similar to saline-treated control animals.

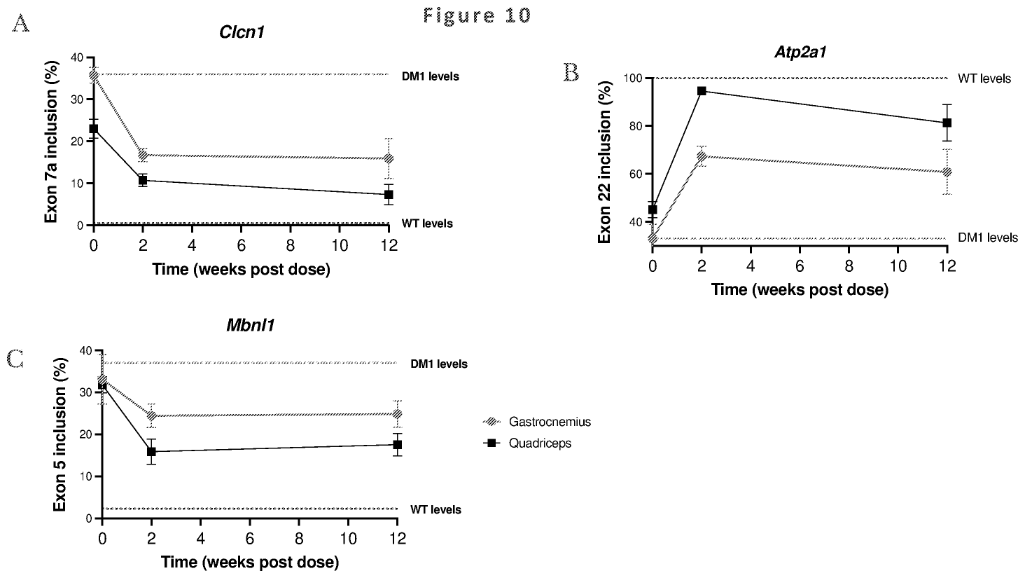

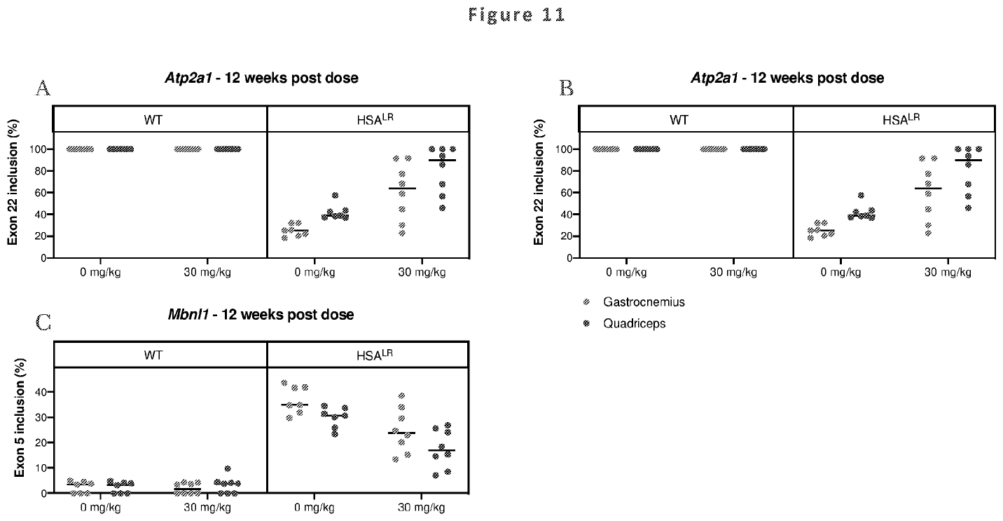

Additionally, we found that PPMO conjugate sustains molecular corrections for three months following a single dose (FIGS. 10-12). This finding provides a basis for the opportunity to use relatively infrequent dosing, which may increase patient convenience and compliance.

### Materials and Methods

### Reagents and General Methods

9-Fluorenylmethoxycarbonyl (Fmoc) protected L-amino acids and Fmoc-β-Ala-OH preloaded Wang resin (0.19 mmol g-1) were obtained from Merck (Hohenbrunn, Germany). HPLC grade acetonitrile, methanol, and synthesis grade N-methyl-2-pyrrolidone (NMP) were purchased from Fisher Scientific (Loughborough, UK). Peptide synthesis grade N,N-dimethylformamide (DMF), benzotriazole-1-yl-oxy-tris-pyrrolidino-phosphonium (PyBOP) and diethyl ether were obtained from AGTC Bioproducts (Yorkshire, UK). Piperidine and trifluoroacetic acid (TFA) were obtained from Alfa Aesar (Heysham, England). PMO was purchased from Gene Tools Inc. (Philomath, USA). All other reagents were obtained from Sigma-Aldrich (St. Louis, Mo., USA) unless otherwise stated. MALDI-TOF mass spectrometry was carried out using a Microflex banch top MALDI-ToF (Bruker). A stock solution of 10 mg mL-1 of a-cyano-4-hydroxycinnamic acid or sinapinic acid in 60% acetonitrile in water containing 0.1% TFA was used as a matrix.

### Synthesis of Peptide on 100 μmol Scale

Peptides were synthesized on a 100 μmol scale using a CEM LibertyBlue™ microwave Peptide Synthesizer (Buckingham, UK) and Fmoc chemistry following manufacturer's recommendations. The side chain protecting groups used were labile to trifluoroacetic acid treatment and the peptide was synthesized using a 5-fold excess of Fmoc-protected amino acids (0.25 mmol) that were activated using PyBOP (5-fold excess) in the presence of DIPEA or with DICIOxyma. Piperidine (20% v/v in DMF) was used to remove N-Fmoc protecting groups. The coupling was carried out once at 75° C. for 5 minutes at 60-watt microwave power except for arginine and the glycosylated amino acid residues, which were coupled twice each.

Histidine and cysteine residues were coupled once at 50° C. for 5 minutes at 60-watt microwave power. Each deprotection reaction was carried out at 75° C. twice, once for 30 seconds and then for 3 minutes at 35-watt microwave power. Once synthesis was complete, the resin was washed with DMF (3×50 mL) and the N-terminus of the solid phase bound peptide was acetylated with acetic anhydride in the presence of DIPEA. The peptide was cleaved from the solid support by treatment with a cleavage cocktail consisting of trifluoroacetic acid (TFA): 3,6-dioxa-1,8-octanedithiol (DODT): H₂O: triisopropylsilane (TIPS) (94%: 2.5%: 2.5%: 1%, 10 mL) or trifluoroacetic acid (TFA): H₂O: m-cresol: triisopropylsilane (TIPS) (94%: 2.5%: 2.5%: 1%, 1 mL) or trifluoroacetic acid (TFA): H₂O: triisopropylsilane (TIPS) (96.5%: 2.5%: 1%, 1 mL) for 2-3 hours at room temperature. Excess TFA was removed by blowing N₂ through the peptide solution. The cleaved peptide was precipitated via the addition of ice-cold diethyl ether and centrifuged at 3000 rpm for 5 minutes. The peptide pellet was washed in ice-cold diethyl ether thrice. The crude peptide was dissolved in water, analyzed and purified by RP-HPLC on Phenomenex Jupiter column (21.2×250 mm, C18, 10 μm) at a flow rate of 20 mL/minute with the following gradient (A: 0.1% TFA, B: 90% CH₃CN, 0.1% TFA) 0-2 minutes 5% B 2-35 minutes 5%-60% B 35-40 minutes 60%-90% B used. The fractions containing the desired peptide were combined and lyophilized to give the product as a white solid.

### Quantification and Reconstitution of PPMO

The PPMO was dissolved in RNase-free water. From this solution, an aliquot was diluted 100-fold in 0.1 M HCl and measured via UV-VIS at 265 nm. The concentration was determined using the Beer-Lambert law: c=(A₂₆₅)/(Ε₂₆₅I)

Prior to use, the PPMO was thawed to room temperature (if frozen beforehand) and vortexed briefly, then incubated for 30 minutes at 37° C. The PPMO aliquot was subsequently sonicated for 5 minutes in a sonicator bath. Finally, the PPMO was briefly vortexed and pulse spun.

The injection solution was prepared by combining the P-PMO at the desired treatment concentration diluted in RNase free water and 9% saline (to a final concentration of 0.9% saline).

### Animal Models and Systemic Administration of PPMO

Experiments were performed in myotonic dystrophy type 1 like mouse strain HSALR mice and FVB control mice. Intravenous injections were performed by single administration via the tail vein in mice aged 8-11 weeks of age. Mice were restrained in an approved apparatus and PPMO administered without anesthetic. Single doses of 10, 20, 30, or 50 mg/kg PPMO were diluted as appropriate in 0.9% saline and administered to HSA^{LR} mice. For control purposes, FVB mice and HSALR mice were administered 0.9% saline. Myotonia was evaluated two weeks post-final administration and subsequently tissues and serum were harvested. Tissues and serum were snap frozen on dry ice and stored at −80° C. or preserved in neutral buffered formalin as appropriate. Animals were sacrificed 12-weeks post a single 30 mg/kg dose for the studies showing lasting effects of PPMO treatment.

In situ Myotonia and Muscle Relaxation Measurement

Isometric contractile properties of gastrocnemius muscle were assessed in situ. Mice were anaesthetized with ketamine (80 mg/kg)/xylazine (15 mg/kg). The knee and foot were fixed with clamps and pins and the distal tendon of the gastrocnemius muscle was attached to a lever arm of a servomoteur system (305B, Dual-Mode Lever). All data was recorded using PowerLab system (4SP, ADlnstruments) and analysed with Chart 4, ADlnstruments software. The sciatic nerve was proximally crushed and stimulated by a bipolar silver electrode using a supramaximal (10-V) square wave pulse of 0.1 ms duration. Absolute maximal isometric tetanic force (P0) was measured during isometric contractions in response to electrical stimulation (frequency of 25 to 150 Hz, train of stimulation of 500 ms). Myotonia was measured as the delay of relaxation muscle after the measure of P0.

RNA Extraction and cDNA Synthesis

Total RNAs were isolated from muscle tissue with TriReagent (Sigma-Aldrich) using Fastprep system and Lysing Matrix D tubes (MP biomedicals) as per manufacturer's protocol. Extracted RNA was reverse transcribed using M-MLV first-strand synthesis system (Life Technologies) according to the manufacturer's instructions. Synthesized cDNA was subsequently used for semi-quantitative PCR analysis according to standard protocol (ReddyMix, Thermo Scientific).

### RT-PCR Analysis

PCR amplification was performed for 25-35 cycles for each gene and PCR products were resolved on 2% agarose gels, ethidium bromide-stained, and quantified using ImageJ software. Quantification of percentage inclusion was determined as a ratio of exon inclusion relative to the total intensity of isoform signals. Primers for RT-PCR are outlined in Table 1. Statistical analysis was performed using GraphPad Prism 8 for macOS Version 8.2.0 (GraphPad Software, Inc.).

TABLE 1
Primers used for RT-PCR analysis

| Transcript | Forward (5′-3′) | Reverse (5′-3′) |
|---|---|---|
| Clcn1 (exon 7a) | GCTGCTGTCC | CTGAATGTGG |
| | TCAGCAAGTT | CTGCAAAGAA |
| | (SEQ ID | (SEQ ID |
| | NO: 91) | NO: 92) |
| Mbnl1 (exon 5) | GCTGCCCAAT | TGGTGGGAGAA |
| | ACCAGGTCAAC | ATGCTGTATGC |
| | (SEQ ID | (SEQ ID |
| | NO: 93) | NO: 94) |
| Atp2a1 (exon 22) | GCTCATGGTC | GGGTCAGTGC |
| | CTCAAGATCT | CTCAGCTTTG |
| | CAC | (SEQ ID |
| | (SEQ ID | NO: 96) |
| | NO: 95) | |

Real Time qPCR Analysis

Real-time qPCR was performed to quantify the mRNA expression with SYBR Green kit (Roche) using a Lightcycler 480 (Roche) as per manufacturer's instructions. PCR cycling conditions were as follows 15-minute denaturing step, 50 cycles of 94° C. for 15 seconds, 58° C. for 20 seconds, and 72° C. for 20 seconds. qPCR data was analyzed with Lightcycler 480 analysis software. Statistical analysis was performed using GraphPad Prism 8 for macOS Version 8.2.0 (GraphPad Software, Inc.).

### In-vitro Cell Culture and P-PMO Treatment

Immortalized myoblasts from a control-individual (Ctrl) or a DM1 patient with 2600 CTG repeats in the 3′ untranscribed region of the DMPK gene (DM1) were cultivated in a proliferation medium consisting of Skeletal Muscle Cell Growth Medium (PromoCell) supplemented with 0.05 mL/mL fetal calf serum (FCS), fetuin 50 μg/mL, 10 ng/mL epidermal growth factor, 1 ng/mL basic fibroblast growth factor, 10 μg/mL insulin, 0.4 μg/mL dexamethasone, and 1% antibiotic antimycotic. Myoblasts were cultured in 5% CO₂ and at 37° C. Cells were passages as required. Cells were assayed on a monthly basis for mycoplasma. All cells used in this study were mycoplasma negative.

For cell viability myoblast treatment, control myoblasts were seeded into a cell culture plate in proliferation media. After 24 hours, myoblasts were treated (gymnotic) with PBS control, unconjugated PMO or PPMO conjugate at a dose range of 0.5-20 μM.

For mis-splice analysis control or DM1 myoblasts were seeded into a cell culture plates in proliferation media. After 24 hours proliferation media was removed and cells were cultured in differentiation media (Skeletal Muscle Cell Growth Medium supplemented with 10 μg/mL insulin and 1% antibiotic antimycotic) for 4 days until myotubes had developed. Then myotubes were treated (gymnotic) with PBS control, unconjugated PMO or PPMO conjugate at a dose range of 1-20 μM, samples were harvested 48 hours after treatment.

### Cell Viability Assay

All treatments were performed in duplicate. Cell viability was assessed from 0 to 48 hours post treatment with PBS control, unconjugated PMO or PPMO conjugate via kinetic cell viability analysis with RealTime-Glo MT Cell Viability Assay (Promega). In brief, MT Cell Viability Substrate and NanoLuc Enzyme were diluted in the appropriate cell culture medium to form the RealTime-Glo reagent. This mixture was added to the cells. Cells were incubated at 37° C. for the duration of the assay and measured every hour for luminescence. Cell viability (percentage) was determined using the formula: [(unconjugated PMO or PPMO conjugate treated cell luminescence)/(PBS treated cell luminescence)]×100

### PMO Quantification

Homogenized tissue lysates from gastrocnemius and quadriceps muscle of VVT and HSALR mice were subject to a customized anion-exchange HPLC based method developed to determine the concentration of PMO oligonucleotide and quantified against a calibration curve. The assay is based on the specific hybridization of an RNA probe (SEQ ID NO: 97-5′-cugcugcugcugcugcugcug-3′) that is complementary in sequence to the PMO and has a fluorescent dye conjugated to both termini. The assay has a linear detection range of 50 ng/g to 5,000 ng/g in mouse tissue.

### Results

The results provided demonstrate a clear dose response effect of the peptide-PMO conjugate on transcript splice correction and on reversal of the myotonia phenotype caused by mis-splicing in the animal model (FIGS. 2-4). These figures also highlight that all of conjugates of the invention demonstrate sufficient efficacy to be considered for therapeutic use. The results further highlight the activity of the peptide-PMO conjugates in vivo in a relevant mouse model of disease, and they suggest that activity of such conjugates is equally effective in quadriceps and gastrocnemius (FIGS. 2-4). These figures demonstrate that PPMO conjugate is able to normalize myotonia and splicing defects in Clcn1, Mbnl1, and Atp2a1 . These results demonstrate a clear dose response with the normalizing effects of myotonia and splice correction being greater following a 30 mg/kg administration, compared to that of a 10 mg/kg administration.

Therefore, the peptide-conjugates of the invention provide promising cell-penetrating peptides for improving the efficacy and reducing the toxicity of therapeutic conjugates for the treatment of neuromuscular disorders in humans.

The results provided demonstrates a clear enhancement in safety and tolerability profiles of the PPMO conjugate (FIG. 5). Increasing doses of PPMO conjugate provide the same level of myoblast cell viability as unconjugated PMO as demonstrated by no apparent cell death in myoblasts up to 48 hours after treatment.

It was also shown that PPMO conjugate dramatically enhances delivery in comparison to the unconjugated PMO and induces more reliable dose-dependent molecular correction than the Pip-conjugated PMO. This data illustrates that PPMO conjugate has a wider therapeutic window and a safer toxicology profile than previous cell penetrating peptide-conjugates such as Pip-conjugated PMO and therefore create a more promising and favorable therapeutic candidate for DM1 patients.

Tissue delivery of PMO after administration of PPMO conjugate was assessed by a probe based fluorescent anion exchange HPLC based method to quantify the delivery of the PMO to key tissue groups. Even at low treatment levels of 10 mg/kg PMO was detected at approximately 17-24 ng/g in muscle tissue, and the levels of PMO detected in muscle increased in a dose-dependent manner.

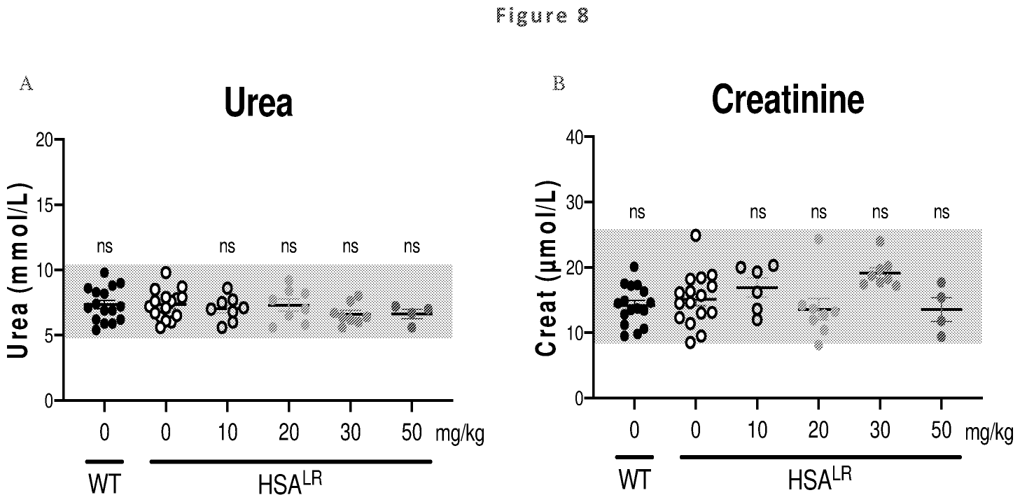

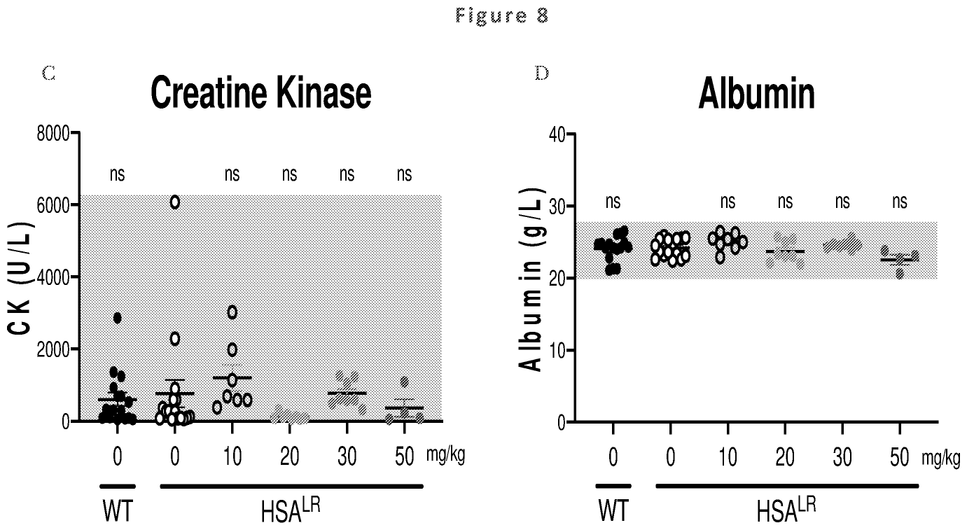

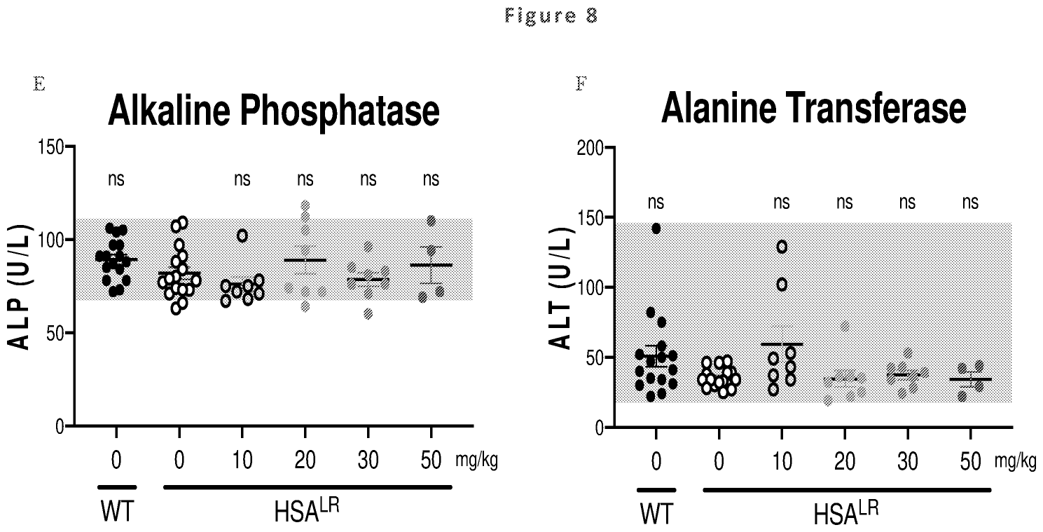

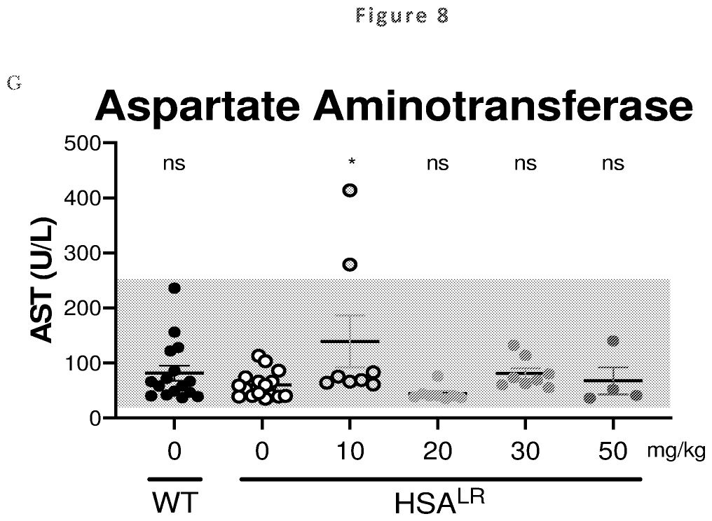

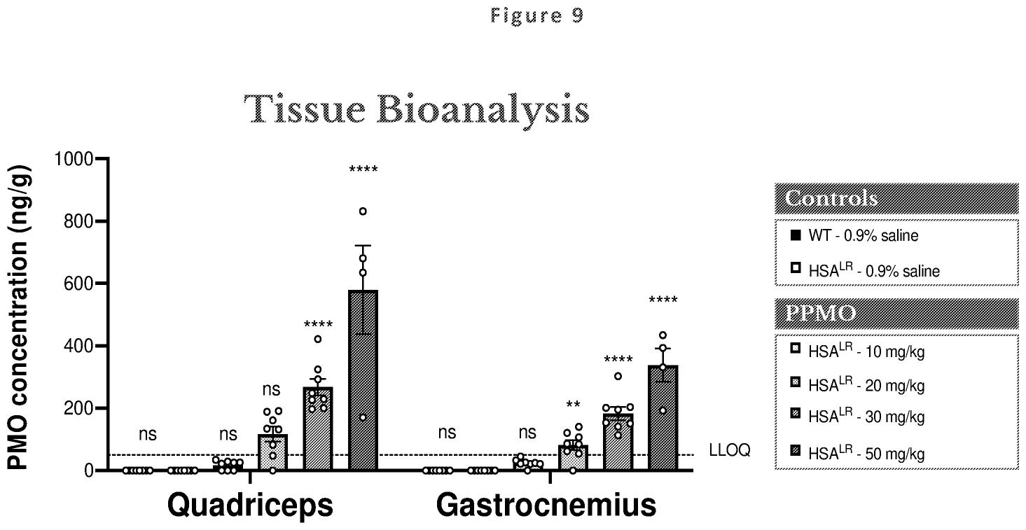

A toxicology evaluation of PPMO conjugate was performed in vivo in VVT and/or HSALR mice. Serum was harvested two weeks post administration of saline or PPMO conjugate and analyzed for urea, creatinine, ALP, ALT, AST, albumin, and CK levels. All clinical chemistry parameters were within the saline control ranges, including at the highest dose level of 50 mg/kg (FIG. 8), indicating a good preliminary safety profile.

Data provided in FIG. 2 and in FIGS. 3a-3c demonstrate the significant impact PPMO conjugate treatment has on targeting the DM1 phenotype by inhibiting the pathological interaction of MBNI1 with the toxic nuclear CUG-expansion through correction of the downstream events of RNA mis-splicing and myotonia.

Evaluation of the physiological correction of the myotonic phenotype present in HSALR mice, a relevant DM1 mouse model, was assessed through myotonia measurement (FIG. 2). A clear dose related correction is induced with treatment with PPMO conjugate, with close to complete correction at 20 mg/kg and statically significant complete correction achieved at doses equal to or higher than 30 mg/kg (FIG. 2).

Molecular abnormalities seen in the DM1 mouse model are corrected by treatment with PPMO conjugate (FIGS. 3a-3c). Treatment with PPMO conjugate provides statically significant correction of key mis-splicing events of Clcn1, Mbnl1, and Atp2a1 transcripts in gastrocnemius and quadriceps muscle following single administration at 10 mg/kg and above.

Treatment with PPMO conjugate has no effect on HSA transcript levels at 20 mg/kg and has no significant effects at higher doses of 30 mg/kg and 50 mg/kg (FIG. 4a and FIG. 4b).

Additionally, we found that PPMO conjugate sustains molecular corrections for months following a single dose (FIGS. 10-12). This surprising finding provides a basis for the opportunity to use relatively infrequent dosing, which may increase patient compliance, comfort, and convenience, as well as minimize the possibility of side effects. Accordingly, the methods described and claimed herein represent substantial advances in the treatment of DM1.

Combined the results provided demonstrate a clear dose response effect of the PPMO conjugate on transcript splice correction in vitro and in vivo as well as on reversal of the myotonic phenotype caused by mis-splicing in the HSALR mouse animal model. Simultaneously PPMO conjugate demonstrates a significantly improved safety profile over Pip-PMO.

### Example 2

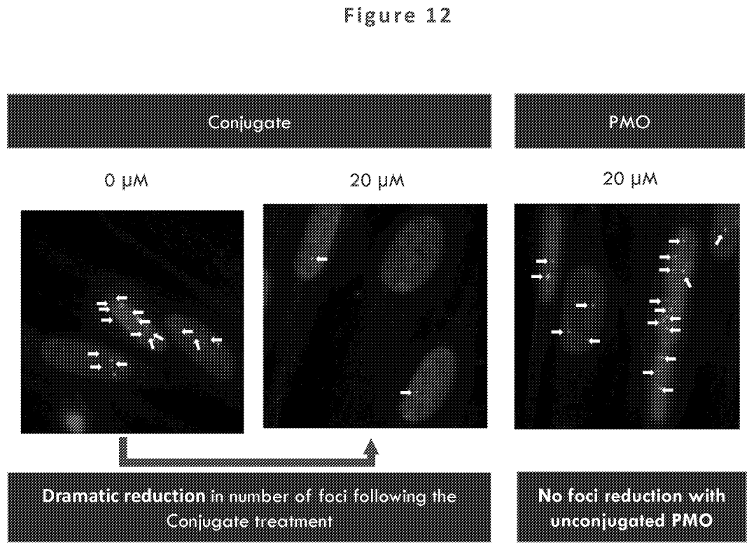

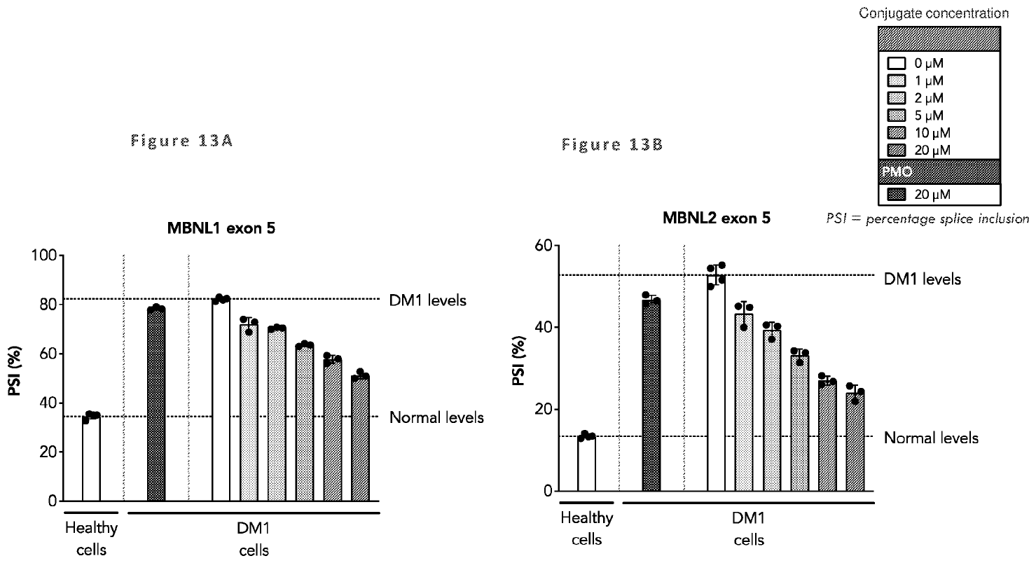

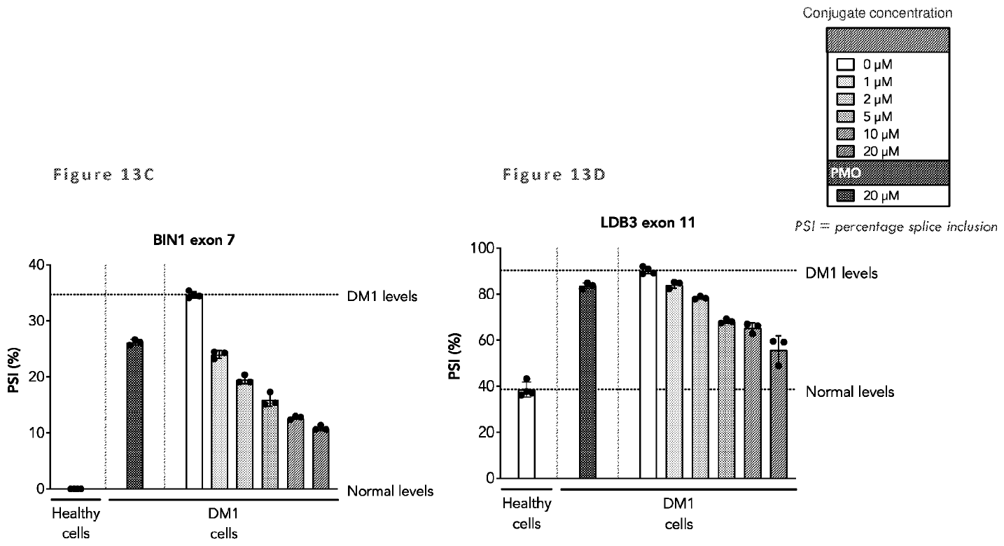

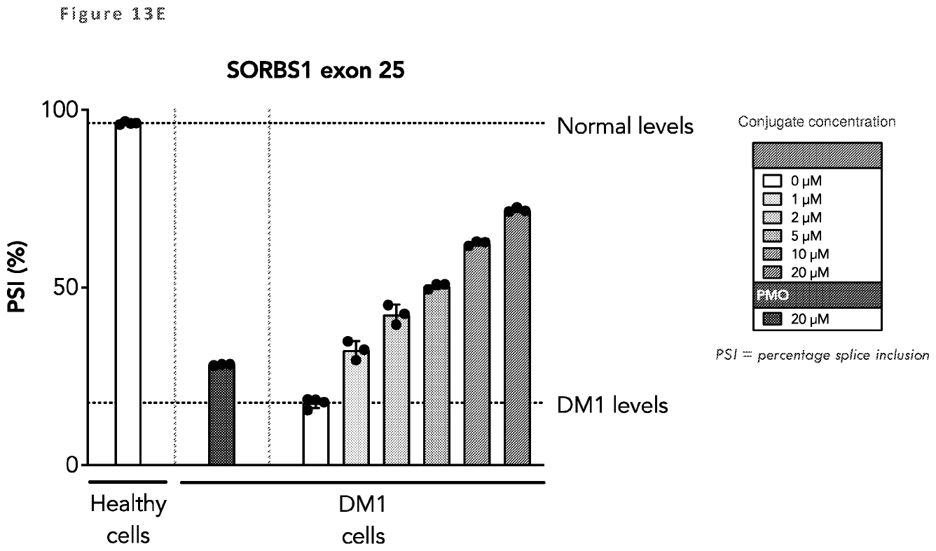

Immortalized myoblasts from healthy individual or DM1 patient with 2600 CTG repeats were cultivated and then differentiated for 4 days. Treatment with unconjugated PMO or peptide-PMO conjugate was carried out at the concentrations given. Cells were harvested for analysis 24 h after treatment. Visualisation was performed with FISH and immunofluorescence. RNA was isolated and analyzed by RT-PCR and capillary electrophoresis (QIAxcel) analysis. The results are shown in FIGS. 12 and 13A-13E.

The results in FIG. 12 demonstrate a dramatic reduction in the number of foci following the conjugate treatment. In contrast, no foci reduction was observed with an unconjugated PMO.

The results in FIGS. 13A-13E demonstrate that the treatment with the conjugate resulted in the MBNL1 liberation and robust correction of downstream mis-splicing.

### Example 3

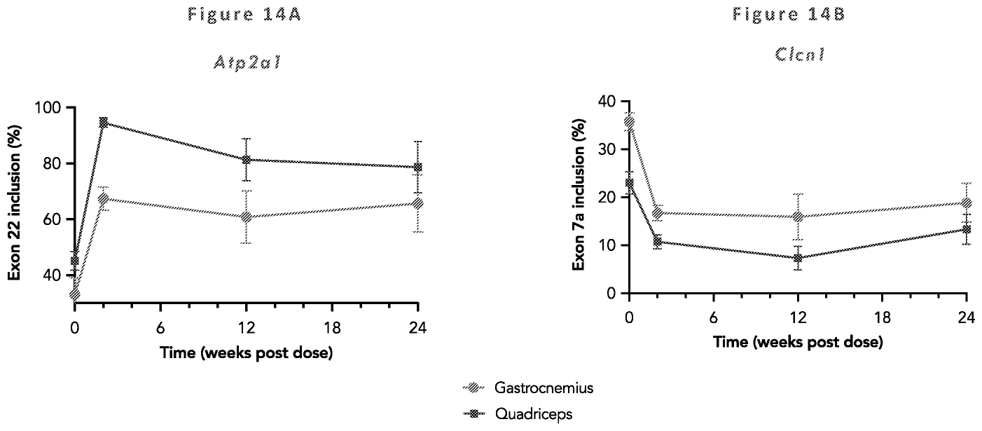

The conjugate described herein was administered intravenously (IV) to a wild-type (WT) mouse and a DM1 mouse model (HSALR) at 30 mg/kg, gastrocnemius and quadriceps muscles were then harvested at 2 weeks (n=8), 12 weeks (n=8), or 24 weeks (n=5) post-administration. Correction of mis-splicing in Atp2a1 and Clcn1 was then assessed. The results are shown in FIGS. 14A and 14B. Conjugate treatment sustained molecular correction for at least 24 weeks following a single dose.

### OTHER EMBODIMENTS

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Some embodiments are within the scope of the following numbered paragraphs.

1. A method of treating a subject having myotonic dystrophy type 1 (DM1), the method comprising administering a therapeutic regimen comprising a plurality of doses of a conjugate spaced at a time interval of at least 1 month, wherein the conjugate comprises an oligonucleotide and a peptide covalently bonded or linked via a linker to the oligonucleotide, the peptide comprising a hydrophobic domain flanked by two cationic domains, each of the

cationic domains comprising one of RBRRBRR (SEQ ID NO: 1), RBRBR (SEQ ID NO: 2), RBRR (SEQ ID NO: 3), RBRRBR (SEQ ID NO: 4), RRBRBR (SEQ ID NO: 5), RBRRB (SEQ ID NO: 6), BRBR (SEQ ID NO: 7), RBHBH (SEQ ID NO: 8), HBHBR (SEQ ID NO: 9), RBRHBHR (SEQ ID NO: 10), RBRBBHR (SEQ ID NO: 11), RBRRBH (SEQ ID NO: 12), HBRRBR (SEQ ID NO: 13), HBHBH (SEQ ID NO: 14), BHBH (SEQ ID NO: 15), BRBSB (SEQ ID NO: 16), BRB[Hyp]B (SEQ ID NO: 17), R[Hyp]H[Hyp]HB (SEQ ID NO: 18), and R[Hyp]RR[Hyp]R (SEQ ID NO: 19), and the hydrophobic domain comprising one of YQFLI (SEQ ID NO: 20), FQILY (SEQ ID NO: 21), ILFQY (SEQ ID NO: 22), FQIY (SEQ ID NO: 23), WWW, WWPWW (SEQ ID NO: 24), WPWW (SEQ ID NO: 25), and WWPW (SEQ ID NO: 26); and the oligonucleotide comprising a total of 12 to 40 contiguous nucleobases, wherein at least 9 contiguous nucleobases are complementary to a CUG repeat sequence.

2. The method of paragraph 1, wherein the time interval is 1 to 6 months.

3. The method of paragraph 1, wherein the time interval is 2 to 6 months.

4. The method of paragraph 1, wherein the time interval is 3 to 6 months.

5. The method of paragraph 1, wherein the time interval is 4 to 6 months.

6. The method of paragraph 1, wherein the time interval is 5 to 6 months.

7. The method of paragraph 1, wherein the time interval is 1 month, 2 months, 3 months, 4 months, 5 months, or 6 months.

8. The method of any one of paragraphs 1 to 7, the therapeutic regimen further comprising a treatment initiation regimen comprising administering the conjugate three or four times at an initiation interval of 2 weeks.

9. The method of any one of paragraphs 1 to 8, wherein the oligonucleotide is 5′-[CAG]ₙ-3′, wherein n is an integer from 5 to 8.

10. The method of paragraph 9, wherein the oligonucleotide is 5′-[CAG]₅-3′.

11. The method of paragraph 9, wherein the oligonucleotide is 5′-[CAG]₆-3′.

12. The method of paragraph 9, wherein the oligonucleotide is 5′-[CAG]₇-3′.

13. The method of paragraph 9, wherein the oligonucleotide is 5′-[CAG]₈-3′.

14. The method of any one of paragraphs 1 to 8, wherein the oligonucleotide is 5′-[AGC]ₙ-3′, wherein n is an integer from 5 to 8.

15. The method of paragraph 14, wherein the oligonucleotide is 5′-[AGC]₅-3′.

16. The method of paragraph 14, wherein the oligonucleotide is 5′-[AGC]₆-3′.

17. The method of paragraph 14, wherein the oligonucleotide is 5′-[AGC]₇-3′.

18. The method of paragraph 14, wherein the oligonucleotide is 5′-[AGC]₈-3′.

19. The method of any one of paragraphs 1 to 8, wherein the oligonucleotide is 5′-[GCA]ₙ-3′, wherein n is an integer from 5 to 8.

20. The method of paragraph 19, wherein the oligonucleotide is 5′-[GCA]₅-3′.

21. The method of paragraph 19, wherein the oligonucleotide is 5′-[GCA]₆-3′.

22. The method of paragraph 19, wherein the oligonucleotide is 5′-[GCA]₇-3′.

23. The method of paragraph 19, wherein the oligonucleotide is 5′-[GCA]₈-3′.

24. The method of any one of paragraphs 1 to 23, wherein the peptide has the following amino acid sequence RBRRBRFQILYBRBR (SEQ ID NO: 35).

25. The method of any one of paragraphs 1 to 23, wherein the peptide has the following amino acid sequence RBRRBRRFQILYRBHBH (SEQ ID NO: 37).

26. The method of any one of paragraphs 1 to 23, wherein the peptide has the following amino acid sequence RBRRBRFQILYRBHBH (SEQ ID NO: 44).

27. The method of any one of paragraphs 1 to 26, wherein the peptide is bonded to the rest of the conjugate through its N-terminus.

28. The method of paragraph 27, wherein the C-terminus of the peptide is —CONH₂.

29. The method of any one of paragraphs 1 to 26, wherein the peptide is bonded to the rest of the conjugate through its C-terminus.

30. The method of paragraph 29, wherein the peptide is acylated at its N-terminus.

31. The method of any preceding paragraph, wherein the conjugate is of the following structure:

[peptide]—[linker]—[oligonucleotide]

32. The method of any one of paragraphs 1 to 30, wherein the conjugate is of the following structure:

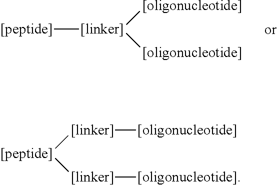

33. The method of any one of paragraphs 1 to 30, wherein the conjugate is of the following structure:

[peptide]—[linker]—[peptide]—[linker]—[oligonucleotide]

34. The method of any preceding paragraph, wherein each linker is independently of formula (I):

T₁—(CR¹R²)ₙ—T₂. (I)

wherein

T₁ is a divalent group for attachment to the peptide and is selected from the group consisting of —NH- and carbonyl;

T₂ is a divalent group for attachment to an oligonucleotide and is selected from the group consisting of —NH- and carbonyl;

n is 1, 2 or 3;

each R¹ is independently -Y¹-X¹-Z¹,

wherein
Y¹ is absent or —(CR^{A1}R^{A2})ₘ-, wherein m is 1, 2, 3 or 4, and R^{A1} and R^{A2} are each independently hydrogen, OH, or (1-2C)alkyl;
X¹ is absent, —O—, —C(O)—, —C(O)O—, —OC(O)—, —CH(OR^{A3})—, —N(R^{A3})—, —N(R^{A3})—C(O)—, —N(R^{A3})—C(O)O—, —C(O)—N(R^{A3})—, —N(R^{A3})C(O)N(R^{A3})—, —N(R^{A3})C(NR^{A3})N(R^{A3})—, —SO—, —S—, —SO₂—, —S(O)₂N(R^{A3})-, or —N(R^{A3})SO₂-, wherein each R^{A3} is independently selected from hydrogen and methyl; and
Z¹ is a further oligonucleotide or is hydrogen, (1-6C)alkyl, (2-6C)alkenyl, (2-6C)alkynyl, aryl, (3-6C)cycloalkyl, (3-6C)cycloalkenyl, or heteroaryl,

wherein each (1 -6C)alkyl, (2-6C)alkenyl, (2-6C)alkynyl, aryl, (3-6C)cycloalkyl, (3-6C)cycloalkenyl, and heteroaryl is optionally substituted with one or more (e.g., 1, 2, 3, 4, or 5) substituent groups selected from the group consisting of (1-4C) alkyl, oxo, halo, cyano, nitro, hydroxy, carboxy, NR^{A4}R^{A5}, and (1-4C)alkoxy, wherein R^{A4} and R^{A5} are each independently selected from the group consisting of hydrogen and (1-4C)alkyl; and

each R² is independently -Y²-X²-Z², wherein
Y² is absent or a group of the formula —[CR^{B1}R^{B2]}ₘ- in which m is an integer selected from 1, 2, 3 or 4, and R^{B1} and R^{B2} are each independently selected from hydrogen, OH or (1-2C)alkyl;
X² is absent, —O—, —C(O)—, —C(O)O—, —OC(O)—, —CH(OR^{B3})—, —N(R^{B3})—, —N(R^{B3})—C(O)—, —N(R^{B3})—C(O)O—, —C(O)—N(R^{B3})—, —N(R^{B3})C(O)N(R^{B3})—, —N(R^{B3})C(NR^{B3})N(R^{B3})—, —SO—, —S— —SO₂—, —S(O)₂N(R^{B3})-, or —N(R^{B3})SO₂-, wherein each R^{B3} is independently selected from hydrogen or methyl; and
Z² is selected from hydrogen, (1 -6C)alkyl, (2-6C)alkenyl, (2-6C)alkynyl, aryl, (3-6C)cycloalkyl, (3-6C)cycloalkenyl or heteroaryl, wherein each (1 -6C)alkyl, (2-6C)alkenyl, (2-6C)alkynyl, aryl, (3-6C)cycloalkyl, (3-6C)cycloalkenyl or heteroaryl is optionally substituted by one or more (e.g., 1, 2, 3, 4, or 5) substituent groups selected from the group consisting of (1-4C) alkyl, oxo, halo, cyano, nitro, hydroxy, carboxy, NR^{B4}R^{B5}, and (1-4C)alkoxy, wherein R^{B4} and R^{B5} are each independently hydrogen or (1-2C)alkyl; with the proviso that; when n=1 and T₁ and T₂ are different to one another, then R¹ and R² are not both H; when n=1, T₁ and T₂ are different to one another and one of R¹ and R² is H then the other of R¹ and R² is not methyl; or when n=2 and each occurrence of R¹ and R² is H, then T₁ and T₂ are both —C(O)- or are both —NH-.

35. The method of paragraph 34, wherein T₂ is —C(O)-.

36. The method of paragraph 34 or 35, wherein each R¹ is independently -Y¹-X¹-Z¹, wherein:

Y¹ is absent or —(CR^{A1}R^{A2})ₘ-, wherein m is 1, 2, 3 or 4, and R^{A1} and R^{A2} are each hydrogen or (1-2C)alkyl;

X¹ is absent, —O—, —C(O)—, —C(O)O—, —N(R^{A3})—, —N(R^{A3})—C(O)—, —C(O)—N(R^{A3})—, —N(R^{A3})C(O)N(R^{A3})—, —N(R^{A3})C(NR^{A3})N(R^{A3})- or —S-, wherein each R^{A3} is independently hydrogen or methyl; and
Z¹ is a further oligonucleotide or is hydrogen, (1-6C)alkyl, (2-6C)alkenyl, (2-6C)alkynyl, aryl, (3-6C)cycloalkyl, (3-6C)cycloalkenyl, or heteroaryl, wherein each (1-6C)alkyl, (2-6C)alkenyl, (2-6C)alkynyl, aryl, (3-6C)cycloalkyl, (3-6C)cycloalkenyl, and heteroaryl is optionally substituted by one or more (e.g., 1, 2, 3, 4, or 5) substituent groups selected from the group consisting of (1-4C) alkyl, oxo, halo, cyano, nitro, hydroxy, carboxy, NR^{A4}R^{A5}, and (1-4C)alkoxy, wherein R^{M} and R^{A5} are each independently hydrogen or (1-2C)alkyl.

37. The method of paragraph 34 or 35, wherein each R¹ is independently -Y¹-X¹-Z¹, wherein:

Y¹ is absent or —(CR^{A1}R^{A2})ₘ-, wherein m is 1, 2, 3, or 4, and R^{A1} and R″ are each independently hydrogen or (1-2C)alkyl;

X¹ is absent, —O—, —C(O)—, —C(O)O—, —N(R^{A3})—, —N(R^{A3})—C(O)—, —C(O)—N(R^{A3})—, —N(R^{A3})C(O)N(R^{A3})—, —N(R^{A3})C(NR^{A3})N(R^{A3})-, or —S-, wherein each R^{A3} is independently hydrogen or methyl; and

Z¹ is a further oligonucleotide or is hydrogen, (1-6C)alkyl, aryl, (3-6C)cycloalkyl, or heteroaryl, wherein each (1-6C)alkyl, aryl, (3-6C)cycloalkyl, and heteroaryl is optionally substituted by one or more substituent groups selected from the group consisting of (1-4C) alkyl, halo, and hydroxy.

38. The method of paragraph 34 or 35, wherein each R¹ is independently -Y¹-X¹-Z¹, wherein:

Y¹ is absent or a group of the formula —(CR^{A1}R^{A2})ₘ-, wherein m is 1, 2, 3 or 4, and R^{A1} and R^{A2} are each independently hydrogen or (1-2C)alkyl;

X¹ is absent, —C(O)—, —C(O)O—, —N(R^{A3})—C(O)—, —C(O)—N(R^{A3})-, wherein each R^{A3} is hydrogen or methyl; and

Z¹ is a further oligonucleotide or is hydrogen, (1-6C)alkyl, aryl, (3-6C)cycloalkyl, or heteroaryl, wherein each (1-6C)alkyl, aryl, (3-6C)cycloalkyl, and heteroaryl is optionally substituted by one or more (e.g., 1, 2, 3, 4, or 5) substituent groups selected from the group consisting of (1-4C) alkyl, halo, and hydroxy.

39. The method of paragraph 34 or 35, wherein each R¹ is independently -Y¹-X¹-Z¹, wherein:
Y¹ is absent, —(CH₂)—, or —(CH₂CH₂)-;
X¹ is absent, —N(R^{A3})—C(O)—, —C(O)—N(R^{A3})-, wherein each R^{A3} is independently hydrogen or methyl; and
Z¹ is hydrogen or (1-2C)alkyl.

40. The method of any one of paragraphs 34 to 39, wherein each R² is independently -Y 2 -Z 2 , wherein Y² is absent or —(CR^{B1}R^{B2})ₘ-, wherein m is 1, 2, 3 or 4, and R^{B1} and R^{B2} are each independently hydrogen or (1-2C)alkyl; and
Z² is hydrogen or (1-6C)alkyl.

41. The method of any one of paragraphs 34 to 39, wherein each R² is hydrogen.

42. The method of any one of paragraphs 34 to 41, wherein n is 2 or 3.

43. The method of any one of paragraphs 34 to 41, wherein n is 1.

44. The method of any one of paragraphs 1 to 43, wherein the linker is an amino acid residue selected from the group consisting of glutamic acid, succinic acid, and gamma-aminobutyric acid residues.

45. The method of any one of paragraphs 1 to 43, wherein the linker is of the following structure:

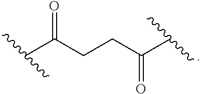 [SMILES: *C(=O)CCC(*)=O]

46. The method of any one of paragraphs 1 to 43, wherein the linker is of the following structure:

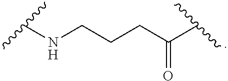 [SMILES: *NCCCC(*)=O]

47. The method of any one of paragraphs 1 to 43, wherein the linker is of the following structure:

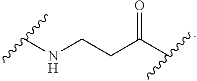 [SMILES: *NCCC(*)=O]

48. The method of any one of paragraphs 1 to 43, wherein the linker is of the following structure:

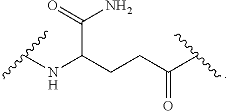 [SMILES: *NC(CCC(*)=O)C(N)=O]

49. The method of any one of paragraphs 1 to 43, wherein the linker is of the following structure:

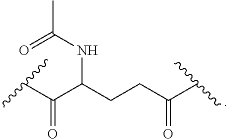 [SMILES: *C(=O)CCC(NC(C)=O)C(*)=O]

50. The method of any one of paragraphs 1 to 43, wherein the conjugate is of the following structure:

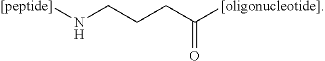

51. The method of any one of paragraphs 1 to 43, wherein the conjugate is of the following structure:

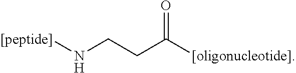

52. The method of any one of paragraphs 1 to 43, wherein the conjugate is of the following structure:

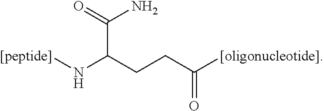

53. The method of any one of paragraphs 1 to 43, wherein the conjugate is of the following structure:

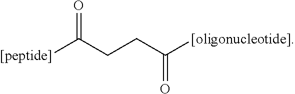

54. The method of any one of paragraphs 1 to 43, wherein the conjugate is of the following structure:

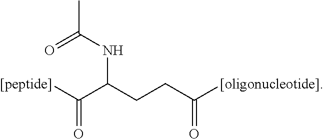

55. The method of any one of paragraphs 1 to 54, wherein the oligonucleotide is bonded to the linker or the peptide at its 3′ terminus.

56. The method of any one of paragraphs 1 to 8, wherein the conjugate is of the following structure:

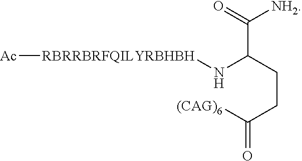

57. The method of any one of paragraphs 1 to 8, wherein the conjugate is of the following structure:

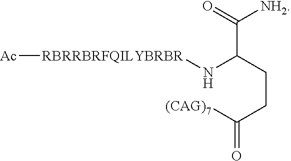

58. The method of any one of paragraphs 1 to 8, wherein the conjugate is of the following structure:

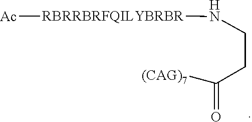

59. The method of any one of paragraphs 1 to 8, wherein the conjugate is of the following structure:

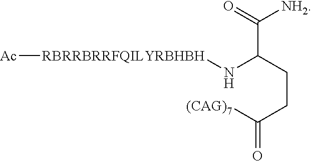

60. The method of any one of paragraphs 1 to 8, wherein the conjugate is of the following structure:

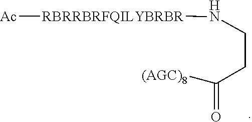

61. The method of any one of paragraphs 1 to 8, wherein the conjugate is of the following structure:

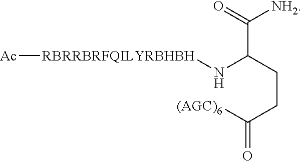

62. The method of any one of paragraphs 1 to 61, wherein the oligonucleotide is a morpholino.

63. The method of 62, wherein all morpholino internucleoside linkages in the morpholino are —P(O)(NMe₂)O-.

64. The method of paragraph 63, wherein the oligonucleotide comprises the following group as its 5′ terminus:

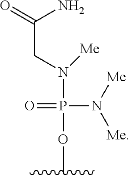 [SMILES: *OP(=O)(N(C)C)N(C)CC(N)=O]

65. The method of any one of paragraphs 1 to 64, wherein the oligonucleotide comprises the following group as its 5′ terminus:

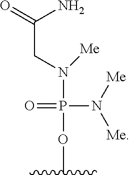 [SMILES: *OP(=O)(N(C)C)N(C)CC(N)=O]

66. The method of any preceding paragraph, wherein the conjugate is administered parenterally.

67. The method of paragraph 66, wherein the conjugate is administered intravenously.

68. The method of any one of paragraphs 1 to 67, wherein each dose within the plurality of doses comprises 5-60 mg/kg of the conjugate.

69. The method of any one of paragraphs 1 to 68, wherein each dose within the plurality of doses comprises 40 mg/kg to 60 mg/kg, 30 mg/kg to 50 mg/kg, 30 mg/kg to 40 mg/kg, 40 mg/kg to 50 mg/kg, 50 mg/kg to 60 mg/kg, 35 mg/kg to 45 mg/kg, 45 mg/kg to 55 mg/kg, 35 mg/kg to 55 mg/kg, 30 10 mg/kg to 45 mg/kg, 35 mg/kg to 50 mg/kg, 40 mg/kg to 55 mg/kg, 45 mg/kg to 60 mg/kg, 1 mg/kg to 30 mg/kg, 1 mg/kg to 20 mg/kg, 5 mg/kg to 25 mg/kg, 10 mg/kg to 30 mg/kg, 1 mg/kg to 15 mg/kg, 5 mg/kg to 20 mg/kg, 10 mg/kg to 25 mg/kg, 15 mg/kg to 30 mg/kg, 1 mg/kg to 10 mg/kg, 5 mg/kg to 15 mg/kg, 10 mg/kg to 20 mg/kg, 15 mg/kg to 25 mg/kg, 20 mg/kg to 30 mg/kg, 1 mg/kg to 25 mg/kg, 4 mg/kg to 20 mg/kg, 6 mg/kg to 15 mg/kg, or 8 mg/kg to 10 mg/kg of the conjugate.

70. The method of paragraph 69, wherein each dose within the plurality of doses comprises 1 mg/kg, 4 mg/kg, 5 mg/kg, 6 mg/kg, 8 mg/kg, 10 mg/kg, 15 mg/kg, 20 mg/kg, 25 mg/kg, 30 mg/kg, 35 mg/kg, 40 mg/kg, 45 mg/kg, 50 mg/kg, or 60 mg/kg of the conjugate.

Other embodiments are within the scope of the claims.

What is claimed is:

1. A method of treating a subject having myotonic dystrophy type 1 (DM1), the method comprising administering a therapeutic regimen comprising a plurality of doses of a conjugate spaced at a time interval of at least 1 month, wherein the conjugate comprises an oligonucleotide and a peptide covalently bonded or linked via a linker to the oligonucleotide, the peptide comprising a hydrophobic domain flanked by two cationic domains, each of the cationic domains comprising one of RBRRBRR (SEQ ID NO: 1), RBRBR (SEQ ID NO: 2), RBRR (SEQ ID NO: 3), RBRRBR (SEQ ID NO: 4), RRBRBR (SEQ ID NO: 5), RBRRB (SEQ ID NO: 6), BRBR (SEQ ID NO: 7), RBHBH (SEQ ID NO: 8), HBHBR (SEQ ID NO: 9), RBRHBHR (SEQ ID NO: 10), RBRBBHR (SEQ ID NO: 11), RBRRBH (SEQ ID NO: 12), HBRRBR (SEQ ID NO: 13), HBHBH (SEQ ID NO: 14), BHBH (SEQ ID NO: 15), BRBSB (SEQ ID NO: 16), BRB[Hyp]B (SEQ ID NO: 17), R[Hyp]H[Hyp]HB (SEQ ID NO: 18), and R[Hyp]RR[Hyp]R (SEQ ID NO: 19), and the hydrophobic domain comprising one of YQFLI (SEQ ID NO: 20), FQILY (SEQ ID NO: 21), ILFQY (SEQ ID NO: 22), FQIY (SEQ ID NO: 23), WWW, WWPWW (SEQ ID NO: 24), WPWW (SEQ ID NO: 25), and WWPW (SEQ ID NO: 26); and
the oligonucleotide comprising a total of 12 to 40 contiguous nucleobases, wherein at least 9 contiguous nucleobases are complementary to a CUG repeat sequence.

2. The method of claim 1, wherein the time interval is 1 to 6 months.

3. The method of claim 1, wherein the time interval is 2 to 6 months.

4. The method of claim 1, wherein the time interval is 3 to 6 months.

5. The method of claim 1, wherein the time interval is 4 to 6 months.

6. The method of claim 1, wherein the time interval is 5 to 6 months.

7. The method of claim 1, wherein the time interval is 1 month, 2 months, 3 months, 4 months, 5 months, or 6 months.

8. The method of any one of claims 1 to 7, the therapeutic regimen further comprising a treatment initiation regimen comprising administering the conjugate three or four times at an initiation interval of 2 weeks.

9. The method of claim 1, wherein the oligonucleotide is 5′-[CAG]ₙ-3′, wherein n is an integer from 5 to 8.

10. The method of claim 9, wherein the oligonucleotide is 5′-[CAG]₅-3′.

11. The method of claim 9, wherein the oligonucleotide is 5′-[CAG]₆-3′.

12. The method of claim 9, wherein the oligonucleotide is 5′-[CAG]₇-3′.

13. The method of claim 9, wherein the oligonucleotide is 5′-[CAG]₈-3′.

14. The method of any one of claims 1 to 7, wherein the oligonucleotide is 5′-[AGC]ₙ-3′, wherein n is an integer from 5 to 8.

15. The method of claim 14, wherein the oligonucleotide is 5′-[AGC]₅-3′.

16. The method of claim 14, wherein the oligonucleotide is 5′-[AGC]₆-3′.

17. The method of claim 14, wherein the oligonucleotide is 5′-[AGC]₇-3′.

18. The method of claim 14, wherein the oligonucleotide is 5′-[AGC]₈-3′.

19. The method of any one of claims 1 to 7, wherein the oligonucleotide is 5′-[GCA]ₙ-3′, wherein n is an integer from 5 to 8.

20. The method of claim 19, wherein the oligonucleotide is 5′-[GCA]₅-3′.

21. The method of claim 19, wherein the oligonucleotide is 5′-[GCA]₆-3′.

22. The method of claim 19, wherein the oligonucleotide is 5′-[GCA]₇-3′.

23. The method of claim 19, wherein the oligonucleotide is 5′-[GCA]₈-3′.

24. The method of any one of claims 1 to 7, wherein the peptide has the following amino acid sequence RBRRBRFQILYBRBR (SEQ ID NO: 35).

25. The method of any one of claims 1 to 7, wherein the peptide has the following amino acid sequence RBRRBRRFQILYRBHBH (SEQ ID NO: 37).

26. The method of any one of claims 1 to 7, wherein the peptide has the following amino acid sequence RBRRBRFQILYRBHBH (SEQ ID NO: 44).

27. The method of any one of claims 1 to 7, wherein the peptide is bonded to the rest of the conjugate through its N-terminus.

28. The method of claim 27, wherein the C-terminus of the peptide is —CONH₂.

29. The method of any one of claims 1 to 7, wherein the peptide is bonded to the rest of the conjugate through its C-terminus.

30. The method of claim 29, wherein the peptide is acylated at its N-terminus.

31. The method of any one of claims 1 to 7, wherein the conjugate is of the following structure:
[peptide]—[linker]—[oligonucleotide]

32. The method of any one of claims 1 to 7, wherein the conjugate is of the following structure:

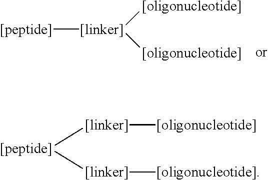

33. The method of any one of claims 1 to 7, wherein the conjugate is of the following structure:
[peptide]—[linker]—[peptide]—[linker]—[oligonucleotide]

34. The method of any one of claims 1 to 7, wherein each linker is independently of formula (I):
T₁—(CR¹R²)ₙ—T₂. (I)
wherein
T₁ is a divalent group for attachment to the peptide and is selected from the group consisting of —NH- and carbonyl;
T₂ is a divalent group for attachment to an oligonucleotide and is selected from the group consisting of —NH- and carbonyl;
n is 1, 2 or 3;
each R¹ is independently -Y¹-X¹-Z¹,
wherein
Y¹ is absent or —(CR^{A1}R^{A2})ₘ-, wherein m is 1, 2, 3 or 4, and R^{A1} and R^{A2} are each independently hydrogen, OH, or (1-2C)alkyl;
X¹ is absent, —O—, —C(O)—, —C(O)O—, —OC(O)—, —CH(OR^{A3})—, —N(R^{A3})—, —N(R^{A3})—C(O)—, —N(R^{A3})—C(O)O—, —C(O)—N(R^{A3})—, —N(R^{A3})C(O)N(R^{A3})—, —N(R^{A3})C(NR^{A3})N(R^{A3})—, —SO—, —S—, —SO₂—, —S(O)₂N(R^{A3})-, or —N(R^{A3})SO₂-, wherein each R^{A3} is independently selected from hydrogen and methyl; and
Z¹ is a further oligonucleotide or is hydrogen, (1-6C)alkyl, (2-6C)alkenyl, (2-6C)alkynyl, aryl, (3-6C)cycloalkyl, (3-6C)cycloalkenyl, or heteroaryl,
wherein each (1 -6C)alkyl, (2-6C)alkenyl, (2-6C)alkynyl, aryl, (3-6C)cycloalkyl, (3-6C)cycloalkenyl, and heteroaryl is optionally substituted with one or more (e.g., 1, 2, 3, 4, or 5) substituent groups selected from the group consisting of (1-4C) alkyl, oxo, halo, cyano, nitro, hydroxy, carboxy, NR^{A4}R^{A5}, and (1-4C)alkoxy, wherein R^{A4} and R^{A5} are each independently selected from the group consisting of hydrogen and (1-4C)alkyl; and
each R² is independently -Y²-X²-Z², wherein
Y² is absent or a group of the formula —[CR^{B1}R^{B2}]ₘ- in which m is an integer selected from 1, 2, 3 or 4, and R^{B1} and R^{B2} are each independently selected from hydrogen, OH or (1-2C)alkyl;
X² is absent, —O—, —C(O)—, —C(O)O—, —OC(O)—, —CH(OR^{B3})—, —N(R^{B3})—, —N(R^{B3})—C(O)—, —N(R^{B3})—C(O)O—, —C(O)—N(R^{B3})—, —N(R^{B3})C(O)N(R^{B3})—, —N(R^{B3})C(NR^{B3})N(R^{B3})—, —SO—, —S— —SO₂—, —S(O)₂N(R^{B3})—, or —N(R^{B3})SO₂-, wherein each R^{B3} is independently selected from hydrogen or methyl; and
Z² is selected from hydrogen, (1 -6C)alkyl, (2-6C)alkenyl, (2-6C)alkynyl, aryl, (3-6C)cycloalkyl, (3-6C)cycloalkenyl or heteroaryl, wherein each (1 -6C)alkyl, (2-6C)alkenyl, (2-6C)alkynyl, aryl, (3-6C)cycloalkyl, (3-6C)cycloalkenyl or heteroaryl is optionally substituted by one or more (e.g., 1, 2, 3, 4, or 5) substituent groups selected from the group consisting of (1-4C) alkyl, oxo, halo, cyano, nitro, hydroxy, carboxy, NR^{B4}R^{B5}, and (1-4C)alkoxy, wherein R^{B4} and R^{B5} are each independently hydrogen or (1-2C)alkyl; with the proviso that; when n=1 and T₁ and T₂ are different to one another, then R¹ and R² are not both H; when n=1, T₁ and T₂ are different to one another and one of R¹ and R² is H then the other of R¹ and R² is not methyl; or when n=2 and each occurrence of R¹ and R² is H, then T₁ and T₂ are both —C(O)- or are both —NH-.

35. The method of claim 34, wherein T₂ is —C(O)-.

36. The method of claim 34, wherein each R¹ is independently -Y¹-X¹-Z¹, wherein:
Y¹ is absent or —(CR^{A1}R^{A2})ₘ-, wherein m is 1, 2, 3 or 4, and R^{A1} and R^{A2} are each hydrogen or (1-2C)alkyl;
X¹ is absent, —O—, —C(O)—, —C(O)O—, —N(R^{A3})—, —N(R^{A3})—C(O)—, —C(O)—N(R³)—, —N(R^{A3})C(O)N(R^{A3})—, —N(R^{A3})C(NR^{A3})N(R^{A3})- or —S-, wherein each R^{A3} is independently hydrogen or methyl; and
Z¹ is a further oligonucleotide or is hydrogen, (1-6C)alkyl, (2-6C)alkenyl, (2-6C)alkynyl, aryl, (3-6C)cycloalkyl, (3-6C)cycloalkenyl, or heteroaryl, wherein each (1-6C)alkyl, (2-6C)alkenyl, (2-6C)alkynyl, aryl, (3-6C)cycloalkyl, (3-6C)cycloalkenyl, and heteroaryl is optionally substituted by one or more (e.g., 1, 2, 3, 4, or 5) substituent groups selected from the group consisting of (1-4C) alkyl, oxo, halo, cyano, nitro, hydroxy, carboxy, NR^{A4}R^{A5}, and (1-4C)alkoxy, wherein R^{M} and R^{A5} are each independently hydrogen or (1-2C)alkyl.

37. The method of claim 34, wherein each R¹ is independently -Y¹-X¹-Z¹, wherein:
Y¹ is absent or —(CR^{A1}R^{A2})ₘ-, wherein m is 1, 2, 3, or 4, and R^{A1} and R″ are each independently hydrogen or (1-2C)alkyl;
X¹ is absent, —O—, —C(O)—, —C(O)O—, —N(R^{A3})—, —N(R^{A3})—C(O)—, —C(O)—N(R^{A3})—, —N(R^{A3})C(O)N(R^{A3})—, —N(R^{A3})C(NR^{A3})N(R^{A3})-, or —S-, wherein each R^{A3} is independently hydrogen or methyl; and
Z¹ is a further oligonucleotide or is hydrogen, (1-6C)alkyl, aryl, (3-6C)cycloalkyl, or heteroaryl, wherein each (1-6C)alkyl, aryl, (3-6C)cycloalkyl, and heteroaryl is optionally substituted by one or more substituent groups selected from the group consisting of (1-4C) alkyl, halo, and hydroxy.

38. The method of claim 34, wherein each R¹ is independently -Y¹-X¹-Z¹, wherein:
Y¹ is absent or a group of the formula —(CR^{A1}R^{A2})ₘ-, wherein m is 1, 2, 3 or 4, and R^{A1} and R^{A2} are each independently hydrogen or (1-2C)alkyl;
X¹ is absent, —C(O)—, —C(O)O—, —N(R^{A3})—C(O)—, —C(O)—N(R^{A3})-, wherein each R^{A3} is hydrogen or methyl; and
Z¹ is a further oligonucleotide or is hydrogen, (1-6C)alkyl, aryl, (3-6C)cycloalkyl, or heteroaryl, wherein each (1-6C)alkyl, aryl, (3-6C)cycloalkyl, and heteroaryl is optionally substituted by one or more (e.g., 1, 2, 3, 4, or 5) substituent groups selected from the group consisting of (1-4C) alkyl, halo, and hydroxy.

39. The method of claim 34, wherein each R¹ is independently -Y¹-X¹-Z¹, wherein:
Y¹ is absent, —(CH₂)-, or —(CH₂CH₂)-;
X¹ is absent, —N(R^{A3})—C(O)—, —C(O)—N(R^{A3})-, wherein each R^{A3} is independently hydrogen or methyl; and
Z¹ is hydrogen or (1-2C)alkyl.

40. The method of claim 34, wherein each R² is independently -Y²-Z²,
wherein Y² is absent or —(CR^{B1}R^{B2})ₘ-, wherein m is 1, 2, 3 or 4, and R^{B1} and R^{B2} are each independently hydrogen or (1-2C)alkyl; and
Z² is hydrogen or (1-6C)alkyl.

41. The method of claim 34, wherein each R² is hydrogen.

42. The method of claim 34, wherein n is 2 or 3.

43. The method of claim 34, wherein n is 1.

44. The method of any one of claims 1 to 7, wherein the linker is an amino acid residue selected from the group consisting of glutamic acid, succinic acid, and gamma-aminobutyric acid residues.

45. The method of any one of claims 1 to 7, wherein the linker is of the following structure:

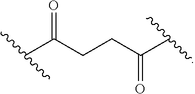

46. The method of any one of claims 1 to 7, wherein the linker is of the following structure:

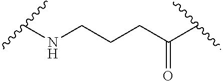

47. The method of any one of claims 1 to 7, wherein the linker is of the following structure:

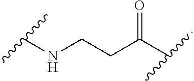

48. The method of any one of claims 1 to 7, wherein the linker is of the following structure:

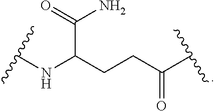 [SMILES: *NC(CCC(*)=O)C(N)=O]

49. The method of any one of claims 1 to 7, wherein the linker is of the following structure:

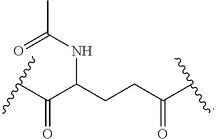 [SMILES: *C(=O)CCC(NC(C)=O)C(*)=O]

50. The method of any one of claims 1 to 7, wherein the conjugate is of the following structure:

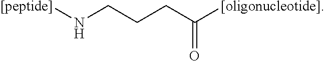

51. The method of any one of claims 1 to 7, wherein the conjugate is of the following structure:

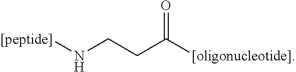

52. The method of any one of claims 1 to 7, wherein the conjugate is of the following structure:

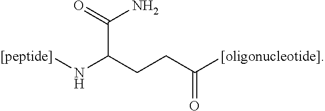

53. The method of any one of claims 1 to 7, wherein the conjugate is of the following structure:

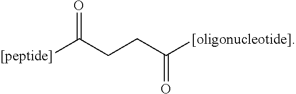

54. The method of any one of claims 1 to 7, wherein the conjugate is of the following structure:

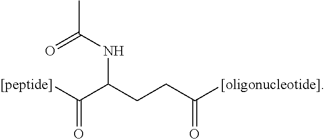

55. The method of any one of claims 1 to 7, wherein the oligonucleotide is bonded to the linker or the peptide at its 3′ terminus.

56. The method of any one of claims 1 to 7, wherein the conjugate is of the following structure:

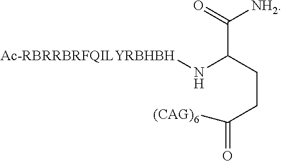

57. The method of any one of claims 1 to 7, wherein the conjugate is of the following structure:

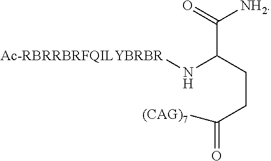

58. The method of any one of claims 1 to 7, wherein the conjugate is of the following structure:

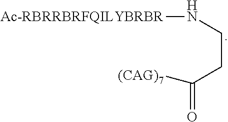

59. The method of any one of claims 1 to 7, wherein the conjugate is of the following structure:

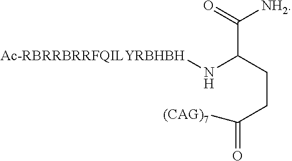

60. The method of any one of claims 1 to 7, wherein the conjugate is of the following structure:

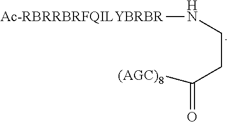

61. The method of any one of claims 1 to 7, wherein the conjugate is of the following structure:

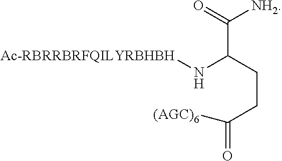

62. The method of any one of claims 1 to 7, wherein the oligonucleotide is a morpholino.

63. The method of 62, wherein all morpholino internucleoside linkages in the morpholino are —P(O)(NMe₂)O-.

64. The method of claim 63, wherein the oligonucleotide comprises the following group as its 5′ terminus:

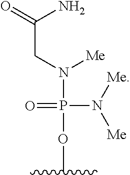 [SMILES: *OP(=O)(N(C)C)N(C)CC(N)=O]

65. The method of any one of claims 1 to 7, wherein the oligonucleotide comprises the following group as its 5′ terminus:

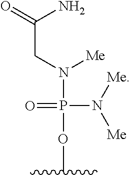 [SMILES: *OP(=O)(N(C)C)N(C)CC(N)=O]

66. The method of any one of claims 1 to 7, wherein the conjugate is administered parenterally.

67. The method of claim 66, wherein the conjugate is administered intravenously.

68. The method of any one of claims 1 to 7, wherein each dose within the plurality of doses comprises 5-60 mg/kg of the conjugate.

69. The method of any one of claims 1 to 7, wherein each dose within the plurality of doses comprises 40 mg/kg to 60 mg/kg, 30 mg/kg to 50 mg/kg, 30 mg/kg to 40 mg/kg, 40 mg/kg to 50 mg/kg, 50 mg/kg to 60 mg/kg, 35 mg/kg to 45 mg/kg, 45 mg/kg to 55 mg/kg, 35 mg/kg to 55 mg/kg, 30 mg/kg to 45 mg/kg, 35 mg/kg to 50 mg/kg, 40 mg/kg to 55 mg/kg, 45 mg/kg to 60 mg/kg, 1 mg/kg to 30 mg/kg, 1 mg/kg to 20 mg/kg, 5 mg/kg to 25 mg/kg, 10 mg/kg to 30 mg/kg, 1 mg/kg to 15 mg/kg, 5 mg/kg to 20 mg/kg, 10 mg/kg to 25 mg/kg, 15 mg/kg to 30 mg/kg, 1 mg/kg to 10 mg/kg, 5 mg/kg to 15 mg/kg, 10 mg/kg to 20 mg/kg, 15 mg/kg to 25 mg/kg, 20 mg/kg to 30 mg/kg, 1 mg/kg to 25 mg/kg, 4 mg/kg to 20 mg/kg, 6 mg/kg to 15 mg/kg, or 8 mg/kg to 10 mg/kg of the conjugate.

70. The method of claim 69, wherein each dose within the plurality of doses comprises 1 mg/kg, 4 mg/kg, 5 mg/kg, 6 mg/kg, 8 mg/kg, 10 mg/kg, 15 mg/kg, 20 mg/kg, 25 mg/kg, 30 mg/kg, 35 mg/kg, 40 mg/kg, 45 mg/kg, 50 mg/kg, or 60 mg/kg of the conjugate.
